SEQUENCE LISTING (115 sequences)

SEQ ID NO: 1 - protein, 506 aa, Cupriavidus necator
MNMAEIAQLGVSNPYKQQYENYIGGAWVPPAGGEYFESTTPITGKPFTRVPRSGQQDVDAALDAAHAAKAAWARTSTTERANILNRIADRIEANLKLLAVAESIDNGKPVRETTAADLPLAVDHFRYFAGCIRAQEGGISEIDADTIAYHFHEPLGVVGQIIPWNFPLLMATWKLAPALAAGNCVVLKPAEQTPASILVLMEVIGDLLPPGVVNVINGFGLEAGKPLASSPRISKVAFTGETTTGRLIMQYASQNLIPVTLELGGKSPNIFFEDVLAADDAFFDKALEGFAMFALNQGEVCTCPSRALIQESIYDRFMERALKRVAAIRQGHPLDTGTMIGAQASAEQLEKILSYIDLGRKEGAQCLTGGERNVLDGDLAGGYYVKPTVFAGHNKMRIFQEEIFGPVVSVTTFKDEEEALAIANDTLYGLGAGVWTRDGARAFRMGRGIQAGRVWTNCYHAYPAHAAFGGYKQSGIGRENHRMMLDHYQQTKNLLVSYSPNALGFF

SEQ ID NO: 2 - DNA, 4228 bp, Artificial Sequence
  synthetic
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatatgaatatggcagaaattgcccagctgggtgtgagtaatccgtataaacagcagtatgaaaattatattggtggtgcatgggttccgccagctggcggtgaatattttgaatcaaccaccccgattaccggcaaaccgtttacccgtgttccgcgtagcggtcagcaggatgtggatgccgcactggatgcagcacatgcagccaaagccgcatgggcacgtacctctaccaccgaacgtgccaatattctgaatcgcattgccgatcgcattgaagccaatctgaaactgctggcagttgccgaatctattgataatggtaaaccggttcgtgaaaccaccgccgccgatctgccgttagcagtggatcattttcgctattttgcaggttgtattcgcgcccaggaaggcggcattagcgaaattgatgcagataccattgcatatcattttcatgaaccgttaggcgttgtgggccagattattccgtggaattttccgctgttaatggcaacctggaaactggccccggccttagcagcaggtaattgtgttgtgctgaaacccgccgaacagaccccggcctcaattctggtgttaatggaagtgattggcgatttactgccgccgggcgttgttaatgtgattaatggctttggcttagaagcaggtaaaccgctggcaagctctccgcgcatttctaaagttgcctttaccggcgaaaccaccaccggtcgtctgattatgcagtatgcaagtcagaatctgattccggtgaccttagaactgggtggtaaaagtccgaatattttttttgaagatgtgctggccgccgatgatgccttttttgataaagccctggaaggctttgccatgtttgcactgaatcagggcgaagtttgtacctgtccgtcacgcgcactgattcaggaatcaatttatgatcgctttatggaacgcgccttaaaacgggttgcagcaattcgtcagggccatccgttagataccggtaccatgattggcgcacaggcctctgccgaacagttagaaaaaattctgagctatattgatctgggtcgcaaagaaggcgcccagtgtctgaccggcggtgaacgtaatgtgctggatggcgatttagccggtggctattatgttaaaccgaccgtgtttgcaggtcataataaaatgcgcatttttcaggaagaaatttttggtccggttgtgagcgtgaccacctttaaagatgaagaagaagccttagccattgccaatgataccctgtatggtttaggtgcaggcgtgtggacccgcgatggtgcacgcgcctttcgtatgggtcgtggtattcaggcaggtcgcgtttggaccaattgttatcatgcctatccggcacatgcagcctttggcggctataaacagagcggtattggtcgcgaaaatcatcgtatgatgttagatcattatcagcagaccaaaaatctgttagtgtcttatagtccgaatgccctgggctttttttaaggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 3 - DNA, 68 bp, Artificial Sequence
  Synthetic
ctagagtctcgagttgacagctagctcagtcctaggtataatgctagcctcgaggaaagaggagaaag

SEQ ID NO: 4 - DNA, 68 bp, Artificial Sequence
  Synthetic
ctagagtctcgagtttacagctagctcagtcctaggtattatgctagcctcgaggaaagaggagaaag

SEQ ID NO: 5 - DNA, 68 bp, Artificial Sequence
  Synthetic
ctagagtctcgagctgacagctagctcagtcctaggtataatgctagcctcgaggaaagaggagaaag

SEQ ID NO: 6 - DNA, 3958 bp, Artificial Sequence
  Synthetic
ccacctgacgtctaagaaaccattattatcatgacattaacctataaaaataggcgtatcacgaggcagaatttcagataaaaaaaatccttagctttcgctaaggatgatttctggaattcgcggccgcatttgtttatggatgcgctggggttgtcgatggcgctcggtacgtttattgcgggtgtgctactggcggaaagtgaatatcgccatgaactggaaacggctatcgatcccttcaaaggcttgctgctcggtttgttctttatctctgtcggcatgtcgctcaacctcggggtgctttatacccatctgttgtgggtagtgataagtgtggttgtgctggtggcggtgaaaattctcgtgctgtatctgctggcgcgattgtatggcgtgcgcagttctgagcggatgcagtttgctggcgtgttgagtcagggcggtgagtttgcctttgtcctcttttctaccgcttcttcacaacgcttattccagggcgaccagattctagagtctcgagttgacagctagctcagtcctaggtataatgctagcctcgaggaaagaggagaaaggtctcaatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcgcgcatggtatggatgaactgtacagatgataacgctgatagtgctagtgtagatcgctactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagaagcggccgctgcagttgcatattcttgcgcgagcgcgcggacgtgtggaagcgcatgagttattacaggcaggggtgacgcagttttcccgtgaaacattctccagtgcgttagagctggggcgcaagacgctggtcacgcttggcatgcatccgcatcaggcgcagcgcgcgcaactgcattttcgccgcctggatatgcgaatgctgcgggagttaatcccgatgcatgctgataccgtacaaatttctcgcgccagggaagcccgacgtgaactggaagagattttccagcgtgaaatgcaacaagaacgacgccagctggacggctgggatgaatttgagttgcaggaattcaaaaaaagcaccgactcggtgccactttttcaagttgataacggactagccttattttaacttgctatttctagctctaaaacgcttcatagtgtcttccggcactagtattatacctaggactgagctagctgtcaaggatccagcatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgtgacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 7 - DNA, 2489 bp, Artificial Sequence
  Synthetic
ttgtttatggatgcgctggggttgtcgatggcgctcggtacgtttattgcgggtgtgctactggcggaaagtgaatatcgccatgaactggaaacggctatcgatcccttcaaaggcttgctgctcggtttgttctttatctctgtcggcatgtcgctcaacctcggggtgctttatacccatctgttgtgggtagtgataagtgtggttgtgctggtggcggtgaaaattctcgtgctgtatctgctggcgcgattgtatggcgtgcgcagttctgagcggatgcagtttgctggcgtgttgagtcagggcggtgagtttgcctttgtcctcttttctaccgcttcttcacaacgcttattccagggcgaccagattctagagtctcgagttgacagctagctcagtcctaggtataatgctagcctcgaggaaagaggagaaagaagcttatgaatatggcagaaattgcccagctgggtgtgagtaatccgtataaacagcagtatgaaaattatattggtggtgcatgggttccgccagctggcggtgaatattttgaatcaaccaccccgattaccggcaaaccgtttacccgtgttccgcgtagcggtcagcaggatgtggatgccgcactggatgcagcacatgcagccaaagccgcatgggcacgtacctctaccaccgaacgtgccaatattctgaatcgcattgccgatcgcattgaagccaatctgaaactgctggcagttgccgaatctattgataatggtaaaccggttcgtgaaaccaccgccgccgatctgccgttagcagtggatcattttcgctattttgcaggttgtattcgcgcccaggaaggcggcattagcgaaattgatgcagataccattgcatatcattttcatgaaccgttaggcgttgtgggccagattattccgtggaattttccgctgttaatggcaacctggaaactggccccggccttagcagcaggtaattgtgttgtgctgaaacccgccgaacagaccccggcctcaattctggtgttaatggaagtgattggcgatttactgccgccgggcgttgttaatgtgattaatggctttggcttagaagcaggtaaaccgctggcaagctctccgcgcatttctaaagttgcctttaccggcgaaaccaccaccggtcgtctgattatgcagtatgcaagtcagaatctgattccggtgaccttagaactgggtggtaaaagtccgaatattttttttgaagatgtgctggccgccgatgatgccttttttgataaagccctggaaggctttgccatgtttgcactgaatcagggcgaagtttgtacctgtccgtcacgcgcactgattcaggaatcaatttatgatcgctttatggaacgcgccttaaaacgggttgcagcaattcgtcagggccatccgttagataccggtaccatgattggcgcacaggcctctgccgaacagttagaaaaaattctgagctatattgatctgggtcgcaaagaaggcgcccagtgtctgaccggcggtgaacgtaatgtgctggatggcgatttagccggtggctattatgttaaaccgaccgtgtttgcaggtcataataaaatgcgcatttttcaggaagaaatttttggtccggttgtgagcgtgaccacctttaaagatgaagaagaagccttagccattgccaatgataccctgtatggtttaggtgcaggcgtgtggacccgcgatggtgcacgcgcctttcgtatgggtcgtggtattcaggcaggtcgcgtttggaccaattgttatcatgcctatccggcacatgcagcctttggcggctataaacagagcggtattggtcgcgaaaatcatcgtatgatgttagatcattatcagcagaccaaaaatctgttagtgtcttatagtccgaatgccctgggctttttttaagagctcgatagtgctagtgtagatcgctactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagaagcggccgctgcagttgcatattcttgcgcgagcgcgcggacgtgtggaagcgcatgagttattacaggcaggggtgacgcagttttcccgtgaaacattctccagtgcgttagagctggggcgcaagacgctggtcacgcttggcatgcatccgcatcaggcgcagcgcgcgcaactgcattttcgccgcctggatatgcgaatgctgcgggagttaatcccgatgcatgctgataccgtacaaatttctcgcgccagggaagcccgacgtgaactggaagagattttccagcgtgaaatgcaacaagaacgacgccagctggacggctgggatgaatttgagt

SEQ ID NO: 8 - DNA, 7177 bp, Artificial Sequence
  Synthetic
ccacctgacgtctaagaaaccattattatcatgacattaacctataaaaataggcgtatcacgaggcagaatttcagataaaaaaaatccttagctttcgctaaggatgatttctggaattcgcggccgcatttccgcagccagcgattatcgcgccgcagcgcaacgcattctgccgccgttcctgttccactatatggatgggggggcatattctgaatacacgctgcgccgcaacgtggaagatttgtcagaagtggcgctgcgccagcgtattctgaaaaacatgtctgacttaagcctggaaacgacgctgtttaatgagaaattgtcgatgccggtggcgctaggtccggtaggtttgtgtggcatgtatgcgcgacgcggcgaagttcaggctgccaaagcagcagatgcgcatggcattccgtttactctctcgacggtttccgtttgcccgattgaagaagtggctccggctatcaaacgtccgatgtggttccagctttatgtgctgcgcgatcgcggctttatgcgtaacgccctggagcgagcaaaagccgcgggttgttcgacgctggttttcacctctagagtctcgagtttatagctagctcagcccttggtacaatgctagcctcgaggaaagaggagaaaggtctcaatgggtaaaataattggtatcgacctgggtactaccaactcttgtgtagcgattatggatggcaccactcctcgcgtgctggagaacgccgaaggcgatcgcaccacgccttctatcattgcctatacccaggatggtgaaactctagttggtcagccggctaaacgtcaggcagtgacgaacccgcaaaacactctgtttgcgattaaacgcctgattggtcgccgcttccaggacgaagaagtacagcgtgatgtttccatcatgccgttcaaaattattgctgctgataacggcgacgcatgggtcgaagttaaaggccagaaaatggcaccgccgcagatttctgctgaagtgctgaaaaaaatgaagaaaaccgctgaagattacctgggtgaaccggtaactgaagctgttatcaccgtaccggcatactttaacgatgctcagcgtcaggcaaccaaagacgcaggccgtatcgctggtctggaagtaaaacgtatcatcaacgaaccgaccgcagctgcgctggcttacggtctggacaaaggcactggcaaccgtactatcgcggtttatgacctgggtggtggtactttcgatatttctattatcgaaatcgacgaagttgacggcgaaaaaaccttcgaagttctggcaaccaacggtgatacccacctggggggtgaagacttcgacagccgtctgatcaactatctggttgaagaattcaagaaagatcagggcattgacctgcgcaacgatccgctggcaatgcagcgcctgaaagaagcggcagaaaaagcgaaaatcgaactgtcttccgctcagcagaccgacgttaacctgccatacatcactgcagacgcgaccggtccgaaacacatgaacatcaaagtgactcgtgcgaaactggaaagcctggttgaagatctggtaaaccgttccattgagccgctgaaagttgcactgcaggacgctggcctgtccgtatctgatatcgacgacgttatcctcgttggtggtcagactcgtatgccaatggttcagaagaaagttgctgagttctttggtaaagagccgcgtaaagacgttaacccggacgaagctgtagcaatcggtgctgctgttcagggtggtgttctgactggtgacgtaaaagacgtactgctgctggacgttaccccgctgtctctgggtatcgaaaccatgggcggtgtgatgacgacgctgatcgcgaaaaacaccactatcccgaccaagcacagccaggtgttctctaccgctgaagacaaccagtctgcggtaaccatccatgtgctgcagggtgaacgtaaacgtgcggctgataacaaatctctgggtcagttcaacctagatggtatcaacccggcaccgcgcggcatgccgcagatcgaagttaccttcgatatcgatgctgacggtatcctgcacgtttccgcgaaagataaaaacagcggtaaagagcagaagatcaccatcaaggcttcttctggtctgaacgaagatgaaatccagaaaatggtacgcgacgcagaagctaacgccgaagctgaccgtaagtttgaagagctggtacagactcgcaaccagggcgaccatctgctgcacagcacccgtaagcaggttgaagaagcaggcgacaaactgccggctgacgacaaaactgctatcgagtctgcgctgactgcactggaaactgctctgaaaggtgaagacaaagccgctatcgaagcgaaaatgcaggaactggcacaggtttcccagaaactgatggaaatcgcccagcagcaacatgcccagcagcagactgccggtgctgatgcttctgcaaacaacgcgaaagatgacgatgttgtcgacgctgaatttgaagaagtcaaagacaaaaaataatcgccctataaacgggtaattatactgacacgggcgaaggggaatttcctccccgcccgtgcattcatctaggggcaatttaaaaaagatggctaagcaagattattacgagattttaggcgtttccaaaacagcggaagagcgtgaaatcagaaaggcctacaaacgcctggccatgaaataccacccggaccgtaaccagggtgacaaagaggccgaggcgaaatttaaagagatcaaggaagcttatgaagttctgaccgactcgcaaaaacgtgcggcatacgatcagtatggtcatgctgcgtttgagcaaggtggcatgggcggcggcggttctggcggcggcgcagacttcagcgatatttttggtgacgttttcggcgatatttttggcggcggacgtggtcgtcaacgtgcggcgcgcggtgctgatttacgctataacatggagctcaccctcgaagaagctgtacgtggcgtgaccaaagagatccgcattccgactctggaagagtgtgacgtttgccacggtagcggtgcaaaaccaggtacacagccgcagacttgtccgacctgtcatggttctggtcaggtgcagatgcgccagggattcttcgctgtacagcagacctgtccacactgtcagggccgcggtacgctgatcaaagatccgtgcaacaaatgtcatggtcatggtcgtgttgagcgcagcaaaacgctgtccgttaaaatcccggcaggggtggacactggagaccgcatccgtcttgcgggcgaaggtgaagcgggcgagcatggcgcaccggcaggcgatctgtacgttcaggttcaggttaaacagcacccgattttcgagcgtgaaggcaacaacctgtattgcgaagtcccgatcaacttcgctatggcggcgctgggtggcgaaatcgaagtaccgacccttgatggtcgcgtcaaactgaaagtgcctggcgaaacccagaccggtaagctattccgtatgcgcggtaaaggcgtcaagtctgtccgcggtggcgcgcagggtgatttgctgtgccgcgttgtcgtcgaaacaccggtaggcctgaacgaaaggcagaaacagctgctgcaagagctgcaagaaagcttcggtggcccaaccggcgagcacaacagcccgcgctcaaagagcttctttgatggtgtgaagaagttttttgacgacctgacccgctaacctcgcggagaaattcatgagtagtaaagaacagaaaacgcctgaggggcaagccccggaagaaattatcatggatcagcacgaagagattgaggcagttgagccagaagcttctgctgagcaggtggatccgcgcgatgaaaaagttgcgaatctcgaagctcagctggctgaagcccagacccgtgaacgtgacggcattttgcgtgtaaaagccgaaatggaaaacctgcgtcgtcgtactgaactggatattgaaaaagcccacaaattcgcgctggagaaattcatcaacgaattgctgccggtgattgatagcctggatcgtgcgctggaagtggctgataaagctaacccggatatgtctgcgatggttgaaggcattgagctgacgctgaagtcgatgctggatgttgtgcgtaagtttggcgttgaagtgatcgccgaaactaacgtcccactggacccgaatgtgcatcaggccatcgcaatggtggaatctgatgacgttgcgccaggtaacgtactgggcattatgcagaagggttatacgctgaatggtcgtacgattcgtgcggcgatggttactgtagcgaaagcaaaagcttaataacgctgatagtgctagtgtagatcgctactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagaagcggccgctgcagatccgcgatttctgggatggcccgatggtgatcaaagggatcctcgatccggaagatgcgcgcgatgcagtacgttttggtgctgatggaattgtggtttctaaccacggtggccgccagttagatggcgtactctcttctgctcgtgcactgcctgctattgcggatgcggtgaaaggtgatatcgccattctggcggatagcggaatacgtaacgggcttgatgtcgtgcgtatgattgcgctcggtgccgacaccgtactgctgggtcgtgctttcctgtatgcactggcaacagcgggccaggcgggtgtagctaatctgctaaatctgatcgaaaaagagatgaaagtggcgatgacgctgactggcgcgaaatcgattagcgaaattacgcaagattcgctggtgcaggggctgggtaaagagttgcctgcggcatgcaggaattcaaaaaaagcaccgactcggtgccactttttcaagttgataacggactagccttattttaacttgctatttctagctctaaaacagaatgcgcatcacgataacactagtattatacctaggactgagctagctgtcaaggatccagcatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgtgacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 9 - DNA, 6725 bp, Artificial Sequence
  Synthetic
ccacctgacgtctaagaaaccattattatcatgacattaacctataaaaataggcgtatcacgaggcagaatttcagataaaaaaaatccttagctttcgctaaggatgatttctggaattcgcggccgcatctgctgatgcacacaccaaccgtattaacagaaaccgtcgccgatacgctgatggcgctggtgttgtctaccgctcgtcgggttgtggaggtagcagaacgggtaaaagcaggcgaatggaccgcgagcataggcccggactggtacggcactgacgttcaccataaaacactgggcattgtcgggatgggacggatcggcatggcgctggcacaacgtgcgcactttggcttcaacatgcccatcctctataacgcgcgtcgccaccataaagaagcagaagaacgcttcaacgcccgctactgcaatttggatactctgttacaagagtcagatttcgtttgcctgatcctgccgttaacggatgagacgcatcatctgtttggcgcagaacaattcgccaaaatgaaatcctccgccattttcattaatgccggacgtggcccggtggttgatgaaaatgcactgattgctgcattgcagaaaggggaaattcacgccgtctagagtctcgagtttatagctagctcagcccttggtacaatgctagcctcgaggaaagaggagaaaggtctcaatgaatattcgtccattgcatgatcgcgtgatcgtcaagcgtaaagaagttgaaactaaatctgctggcggcatcgttctgaccggctctgcagcggctaaatccacccgcggcgaagtgctggctgtcggcaatggccgtatccttgaaaatggcgaagtgaagccgctggatgtgaaagttggcgacatcgttattttcaacgatggctacggtgtgaaatctgagaagatcgacaatgaagaagtgttgatcatgtccgaaagcgacattctggcaattgttgaagcgtaatccgcgcacgacactgaacatacgaatttaaggaataaagataatggcagctaaagacgtaaaattcggtaacgacgctcgtgtgaaaatgctgcgcggcgtaaacgtactggcagatgcagtgaaagttaccctcggtccaaaaggccgtaacgtagttctggataaatctttcggtgcaccgaccatcaccaaagatggtgtttccgttgctcgtgaaatcgaactggaagacaagttcgaaaatatgggtgcgcagatggtgaaagaagttgcctctaaagcaaacgacgctgcaggcgacggtaccaccactgcaaccgtactggctcaggctatcatcactgaaggtctgaaagctgttgctgcgggcatgaacccgatggacctgaaacgtggtatcgacaaagcggttaccgctgcagttgaagaactgaaagcgctgtccgtaccatgctctgactctaaagcgattgctcaggttggtaccatctccgctaactccgacgaaaccgtaggtaaactgatcgctgaagcgatggacaaagtcggtaaagaaggcgttatcaccgttgaagacggtaccggtctgcaggacgaactggacgtggttgaaggtatgcagttcgaccgtggctacctgtctccttacttcatcaacaagccggaaactggcgcagtagaactggaaagcccgttcatcctgctggctgacaagaaaatctccaacatccgcgaaatgctgccggttctggaagctgttgccaaagcaggcaaaccgctgctgatcatcgctgaagatgtagaaggcgaagcgctggcaactctggttgttaacaccatgcgtggcatcgtgaaagtcgctgcggttaaagcaccgggcttcggcgatcgtcgtaaagctatgctgcaggatatcgcaaccctgactggcggtaccgtgatctctgaagagatcggtatggagctggaaaaagcaaccctggaagacctgggtcaggctaaacgtgttgtgatcaacaaagacaccaccactatcatcgatggcgtgggtgaagaagctgcaatccagggccgtgttgctcagatccgtcagcagattgaagaagcaacttctgactacgaccgtgaaaaactgcaggaacgcgtagcgaaactggcaggcggcgttgcagttatcaaagtgggtgctgctaccgaagttgaaatgaaagagaaaaaagcacgcgttgaagatgccctgcacgcgacccgtgctgcggtagaagaaggcgtggttgctggtggtggtgttgcgctgatccgcgtagcgtctaaactggctgacctgcgtggtcagaacgaagaccagaacgtgggtatcaaagttgcactgcgtgcaatggaagctccgctgcgtcagatcgtattgaactgcggcgaagaaccgtctgttgttgctaacaccgttaaaggcggcgacggcaactacggttacaacgcagcaaccgaagaatacggcaacatgatcgacatgggtatcctggatccaaccaaagtaactcgttctgctctgcagtacgcagcttctgtggctggcctgatgatcaccaccgaatgcatggttaccgacctgccgaaaaacgatgcagctgacttaggcgctgctggcggtatgggcggcatgggtggcatgggcggcatgatgtaattgccctgcacctcgcagaaataaacaaacccccctgtgattttttgaggtaacaagatgcaagtttcagttgaaaccactcaaggccttggccgccgtgtaacgattactatcgctgctgacagcatcgagaccgctgttaaaagcgagctggtcaacgttgcgaaaaaagtacgtattgacggcttccgcaaaggcaaagtgccaatgaatatcgttgctcagcgttatggcgcgtctgtacgccaggacgttctgggtgacctgatgagccgtaacttcattgacgccatcattaaagaaaaaatcaatccggctggcgcaccgacttatgttccgggcgaatacaagctgggtgaagacttcacttactctgtagagtttgaagtttatccggaagttgaactgcagggtctggaagcgatcgaagttgaaaaaccgatcgttgaagtgaccgacgctgacgttgacggcatgctggatactctgcgtaaacagcaggcgacctggaaagaaaaagacggcgctgttgaagcagaagaccgcgtaaccatcgacttcaccggttctgtagacggcgaagagttcgaaggcggtaaagcgtctgatttcgtactggcgatgggccagggtcgtatgatcccgggctttgaagacggtatcaaaggccacaaagctggcgaagagttcaccatcgacgtgaccttcccggaagaataccacgcagaaaacctgaaaggtaaagcagcgaaattcgctatcaacctgaagaaagttgaagagcgtgaactgccggaactgactgcagaattcatcaaacgtttcggcgttgaagatggttccgtagaaggtctgcgcgctgaagtgcgtaaaaacatggagcgcgagctgaagagcgccatccgtaaccgcgttaagtctcaggcgatcgaaggtctggtaaaagctaacgacatcgacgtaccggctgcgctgatcgacagcgaaatcgacgttctgcgtcgccaggctgcacagcgtttcggtggcaacgaaaaacaagctctggaactgccgcgcgaactgttcgaagaacaggctaaacgccgcgtagttgttggcctgctgctgggcgaagttatccgcaccaacgagctgaaagctgacgaagagcgcgtgaaaggcctgatcgaagagatggcttctgcgtacgaagatccgaaagaagttatcgagttctacagcaaaaacaaagaactgatggacaacatgcgcaatgttgctctggaagaacaggctgttgaagctgtactggcgaaagcgaaagtgactgaaaaagaaaccactttcaacgagctgatgaaccagcaggcgtaataacgctgatagtgctagtgtagatcgctactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagaagcggccgctgcagtcgaacaagagccactgtccgtagattcgccgttgctctcaatggccaacgtcgtcgcagtaccgcatattggatctgccactcatgagacgcgttatggcatggccgcctgtgccgtggataatttgattgatgcgttacaaggaaaggttgagaagaactgtgtgaatccgcacgtcgcggactaagccgcgactgcgtggagtaaagcccgataatcgctcgggcttttactctttattgggttgcagtaactgctgtagtccaggcctgattaaacgcctgatgttgtgccggtaatggcgcaatcagtttgttatattcacttgcctgctgtgaagtcgggaacttgcaggaattcaaaaaaagcaccgactcggtgccactttttcaagttgataacggactagccttattttaacttgctatttctagctctaaaacccgggctggatgtcttcgaaactagtattatacctaggactgagctagctgtcaaggatccagcatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgtgacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 10 - DNA, 35 bp, Artificial Sequence
  Synthetic
ttgacagctagctcagtcctaggtataatgctagc

SEQ ID NO: 11 - DNA, 35 bp, Artificial Sequence
  Synthetic
tttacagctagctcagtcctaggtattatgctagc

SEQ ID NO: 12 - DNA, 35 bp, Artificial Sequence
  Synthetic
ttgacagctagctcagtcctaggtactgtgctagc

SEQ ID NO: 13 - DNA, 35 bp, Artificial Sequence
  Synthetic
ctgatagctagctcagtcctagggattatgctagc

SEQ ID NO: 14 - DNA, 35 bp, Artificial Sequence
  Synthetic
tttacagctagctcagtcctagggactgtgctagc

SEQ ID NO: 15 - DNA, 35 bp, Artificial Sequence
  Synthetic
tttacggctagctcagtcctaggtacaatgctagc

SEQ ID NO: 16 - DNA, 35 bp, Artificial Sequence
  Synthetic
tttatggctagctcagtcctaggtacaatgctagc

SEQ ID NO: 17 - DNA, 35 bp, Artificial Sequence
  Synthetic
ttgacagctagctcagtcctagggattgtgctagc

SEQ ID NO: 18 - DNA, 77 bp, Artificial Sequence
  Synthetic
aaagtgttttgtaatcataaagaaatattaaggtggggtaggaatagtataatatgtttattcaaccgaacttaatg

SEQ ID NO: 19 - DNA, 339 bp, Artificial Sequence
  Synthetic
gtaaatttaggattaatcctggaactttttttgtcgcccagccaatgctttcagtcgtgactaattttccttgcggaggcttgtctgaagcggtttccgcgattttcttctgtaaattgtcgctgacaaaaaagattaaacgtaccttatacaagacttttttttcatatgcctgacggagttcacacttgtaagttttcaactacgttgtagactttacatcgccaggggtgctcggcataagccgaagatatcggtagagttaatattgagcagatccccggtgaaggatttaaccgtgttatctcgttggagatattcatggcgtattttggatga

SEQ ID NO: 20 - DNA, 35 bp, Artificial Sequence
  Synthetic
ttgacggctagctcagtcctaggtacagtgctagc

SEQ ID NO: 21 - DNA, 35 bp, Artificial Sequence
  Synthetic
ttgacagctagctcagtcctaggtattgtgctagc

SEQ ID NO: 22 - DNA, 35 bp, Artificial Sequence
  Synthetic
tttacggctagctcagtcctaggtactatgctagc

SEQ ID NO: 23 - DNA, 30 bp, Artificial Sequence
  Synthetic
gtttatacataggcgagtactctgttatgg

SEQ ID NO: 24 - DNA, 30 bp, Artificial Sequence
  Synthetic
ggtttcaaaattgtgatctatatttaacaa

SEQ ID NO: 25 - DNA, 35 bp, Artificial Sequence
  Synthetic
ttgacggctagctcagtcctaggtattgtgctagc

SEQ ID NO: 26 - DNA, 35 bp, Artificial Sequence
  Synthetic
ttgacagctagctcagtcctagggactatgctagc

SEQ ID NO: 27 - DNA, 35 bp, Artificial Sequence
  Synthetic
tttatagctagctcagcccttggtacaatgctagc

SEQ ID NO: 28 - DNA, 35 bp, Artificial Sequence
  Synthetic
ctgatggctagctcagtcctagggattatgctagc

SEQ ID NO: 29 - DNA, 35 bp, Artificial Sequence; identical to SEQ ID NO: 13
  Synthetic

SEQ ID NO: 30 - DNA, 35 bp, Artificial Sequence
  Synthetic
ttgacggctagctcagtcctaggtatagtgctagc

SEQ ID NO: 31 - DNA, 35 bp, Artificial Sequence
  Synthetic
ctgacagctagctcagtcctaggtataatgctagc

SEQ ID NO: 32 - DNA, 35 bp, Artificial Sequence
  Synthetic
tttacggctagctcagccctaggtattatgctagc

SEQ ID NO: 33 - DNA, 35 bp, Artificial Sequence
  Synthetic
tttacggctagctcagtcctaggtatagtgctagc

SEQ ID NO: 34 - DNA, 30 bp, Artificial Sequence
  Synthetic
agaggttccaactttcaccataatgaaaca

SEQ ID NO: 35 - DNA, 30 bp, Artificial Sequence
  Synthetic
caccttcgggtgggcctttctgcgtttata

SEQ ID NO: 36 - DNA, 30 bp, Artificial Sequence
  Synthetic
ggctagctcagtcctaggtactatgctagc

SEQ ID NO: 37 - DNA, 30 bp, Artificial Sequence
  Synthetic
aaagtgtgacgccgtgcaaataatcaatgt

SEQ ID NO: 38 - DNA, 30 bp, Artificial Sequence
  Synthetic
ggctagctcagtcctaggtattatgctagc

SEQ ID NO: 39 - DNA, 30 bp, Artificial Sequence
  Synthetic
taaacaactaacggacaattctacctaaca

SEQ ID NO: 40 - DNA, 178 bp, Artificial Sequence
  Synthetic
aagctgttgtgaccgcttgctctagccagctatcgagttgtgaaccgatccatctagcaattggtctcgatctagcgataggcttcgatctagctatgtatcactcattaggcaccccaggctttacactttatgcttccggctcgtataatgtgtggtgctggttagcgcttgctat

SEQ ID NO: 41 - DNA, 190 bp, Artificial Sequence
  Synthetic
aagctgttgtgaccgcttgctctagccagctatcgagttgtgaaccgatccatctagcaattggtctcgatctagcgataggcttcgatctagctatgtagaaacgccgtgtgctcgatcgcttgataaggtccacgtagctgctataattgcttcaacagaacatattgactatccggtattacccggc

SEQ ID NO: 42 - DNA, 30 bp, Artificial Sequence
  Synthetic
catacgccgttatacgttgtttacgctttg

SEQ ID NO: 43 - DNA, 73 bp, Artificial Sequence
  Synthetic
taattcctaatttttgttgacactctatcgttgatagagttattttaccactccctatcagtgatagagaaaa

SEQ ID NO: 44 - DNA, 44 bp, Artificial Sequence
  Synthetic
ttgacaattaatcatccggctcgtataatgtgtggaattgtgag

SEQ ID NO: 45 - DNA, 52 bp, Artificial Sequence
  Synthetic
cccaggctttacactttatgcttccggctcgtataatgtgtggaattgtgag

SEQ ID NO: 46 - DNA, 30 bp, Artificial Sequence
  Synthetic
tgctagctactagagattaaagaggagaaa

SEQ ID NO: 47 - DNA, 30 bp, Artificial Sequence
  Synthetic
ggctagctcagtcctaggtacagtgctagc

SEQ ID NO: 48 - DNA, 30 bp, Artificial Sequence
  Synthetic
ccccgaaagcttaagaatataattgtaagc

SEQ ID NO: 49 - DNA, 30 bp, Artificial Sequence
  Synthetic
atatatatatatatataatggaagcgtttt

SEQ ID NO: 50 - DNA, 74 bp, Artificial Sequence
  Synthetic
tccctatcagtgatagagattgacatccctatcagtgatagagatactgagcacatcagcaggacgcactgacc

SEQ ID NO: 51 - DNA, 68 bp, Artificial Sequence
  Synthetic
attccactaatttattccatgtcacacttttcgcatctttgttatgctatggttatttcataccataa

SEQ ID NO: 52 - DNA, 76 bp, Artificial Sequence
  Synthetic
ataaatgtgagcggataacaattgacattgtgagcggataacaagatactgagcacatcagcaggacgcactgacc

SEQ ID NO: 53 - DNA, 65 bp, Artificial Sequence
  Synthetic
aaaaacgccgcaaagtttgagcgaagtcaataaactctctacccattcagggcaatatctctctt

SEQ ID NO: 54 - DNA, 81 bp, Artificial Sequence
  Synthetic
atgaagaaaaagatcattagcgcgatcctgatgagcaccgtgattctgagcgcggcggcgccgctgagcggtgtttatgcg

SEQ ID NO: 55 - DNA, 63 bp, Artificial Sequence
  Synthetic
atgaagaaaaccgcgattgcgattgcggtggcgctggcgggtttcgcgaccgttgcgcaggcg

SEQ ID NO: 56 - DNA, 57 bp, Artificial Sequence
  Synthetic
atgaagaaaatctggctggcgctggcgggtctggtgctggcgttcagcgcgagcgcg

SEQ ID NO: 57 - DNA, 57 bp, Artificial Sequence; identical to SEQ ID NO: 56
  Synthetic

SEQ ID NO: 58 - DNA, 63 bp, Artificial Sequence
  Synthetic
atggaaggaaacactcgtgaagacaattttaaacatttattaggtaatgacaatgttaaacgc

SEQ ID NO: 59 - DNA, 147 bp, Artificial Sequence
  Synthetic
atgaataaaatatactcccttaaatatagtgctgccactggcggactcattgctgtttctgaattagcgaaaagagtttctggtaaaacaaaccgaaaacttgtagcaacaatgttgtctctggctgttgccggtacagtaaatgca

SEQ ID NO: 60 - DNA, 90 bp, Artificial Sequence
  Synthetic
atgagcaactggatcaccgacaacaaaccggctgcgatggttgcgggtgtgggcctgctgctgttcctgggtctgagcgcgaccggctac

SEQ ID NO: 61 - DNA, 33 bp, Artificial Sequence
  Synthetic
atgcgtaaaggcgaagagaaggaggttaactga

SEQ ID NO: 62 - DNA, 53 bp, Artificial Sequence
  Synthetic
atgaaagcaattttcgtactgaaacatcttaatcatgctaaggaggttttcta

SEQ ID NO: 63 - DNA, 33 bp, Artificial Sequence
  Synthetic
atgattatgtccggttataaggaggttaactga

SEQ ID NO: 64 - DNA, 45 bp, Artificial Sequence
  Synthetic
atgaaaatcgaagcaggtaaactggtacagaaggaggttaactga

SEQ ID NO: 65 - DNA, 22 bp, Artificial Sequence
  Synthetic
ggaggaaaaattaaaaaagaac

SEQ ID NO: 66 - DNA, 12 bp, Artificial Sequence
  Synthetic
aaagaggagaaa

SEQ ID NO: 67 - skipped

SEQ ID NO: 68 - DNA, 186 bp, Artificial Sequence
  Synthetic
cgagctcgatagtgctagtgtagatcgctactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagaagcggccgctgcag

SEQ ID NO: 69 - DNA, 173 bp, Artificial Sequence
  Synthetic
cgagctcgatagtgctagtgtagatcgctactagagccaggcatcaaataaaacgaaagactgggcctttcgttttatctgttgtttgtcggtgaacgctctctactagagtcacactggctcaccttcgggtgggcctttctgcgtttatatactagaagcggccgctgcag

SEQ ID NO: 70 - DNA, 20 bp, Artificial Sequence
  Synthetic
ggcgagttaacgacgacaca

SEQ ID NO: 71 - DNA, 20 bp, Artificial Sequence
  Synthetic
cgttgaactgggtgtggaat

SEQ ID NO: 72 - DNA, 20 bp, Artificial Sequence
  Synthetic
ctgaaaccgctgcggcgatg

SEQ ID NO: 73 - DNA, 20 bp, Artificial Sequence
  Synthetic
gttcgcagtggaagtaccgt

SEQ ID NO: 74 - DNA, 20 bp, Artificial Sequence
  Synthetic
ctgtgcaaacaagtgtctca

SEQ ID NO: 75 - DNA, 20 bp, Artificial Sequence
  Synthetic
gccggaagacactatgaagc

SEQ ID NO: 76 - DNA, 20 bp, Artificial Sequence
  Synthetic
tcgcacagcgtgtaccacag

SEQ ID NO: 77 - DNA, 20 bp, Artificial Sequence
  Synthetic
gaaggggaagaggcgcgcgt

SEQ ID NO: 78 - DNA, 20 bp, Artificial Sequence
  Synthetic
cggtttagttcacagaagcc

SEQ ID NO: 79 - DNA, 20 bp, Artificial Sequence
  Synthetic
ttgcgtattttcaaaaagcg

SEQ ID NO: 80 - DNA, 20 bp, Artificial Sequence
  Synthetic
tcatcagagtaagtcggata

SEQ ID NO: 81 - DNA, 20 bp, Artificial Sequence
  Synthetic
ctgaccaacgcttctttacc

SEQ ID NO: 82 - DNA, 20 bp, Artificial Sequence
  Synthetic
ccgaagtccctgtgtgcttt

SEQ ID NO: 83 - DNA, 20 bp, Artificial Sequence
  Synthetic
ccctgccactcacaccattc

SEQ ID NO: 84 - DNA, 20 bp, Artificial Sequence
  Synthetic
ccttctcctggcaagcttac

SEQ ID NO: 85 - DNA, 20 bp, Artificial Sequence
  Synthetic
cgacaagaagtacctgcaac

SEQ ID NO: 86 - DNA, 20 bp, Artificial Sequence
  Synthetic
gttatcgtgatgcgcattct

SEQ ID NO: 87 - DNA, 20 bp, Artificial Sequence
  Synthetic
taacacccagcccgatgccc

SEQ ID NO: 88 - DNA, 20 bp, Artificial Sequence
  Synthetic
atattactggaacaacataa

SEQ ID NO: 89 - DNA, 20 bp, Artificial Sequence
  Synthetic
ccgtgacgttatccgcacca

SEQ ID NO: 90 - DNA, 20 bp, Artificial Sequence
  Synthetic
tatcgccttcatccacacga

SEQ ID NO: 91 - DNA, 20 bp, Artificial Sequence
  Synthetic
ccgggctggatgtcttcgaa

SEQ ID NO: 92 - DNA, 20 bp, Artificial Sequence
  Synthetic
gagggtgagccataatgaag

SEQ ID NO: 93 - DNA, 20 bp, Artificial Sequence
  Synthetic
ggatatgtggggtaacgacg

SEQ ID NO: 94 - DNA, 20 bp, Artificial Sequence
  Synthetic
acgttcaggctgctaaagat

SEQ ID NO: 95 - DNA, 48 bp, Artificial Sequence
  Synthetic
cgaggaaagaggagaaagaagcttatgaatatggcagaaattgcccag

SEQ ID NO: 96 - DNA, 48 bp, Artificial Sequence
  Synthetic
atctacactagcactatcgagctcttaaaaaaagcccagggcattcgg

SEQ ID NO: 97 - DNA, 48 bp, Artificial Sequence
  Synthetic
ccgaatgccctgggctttttttaagagctcgatagtgctagtgtagat

SEQ ID NO: 98 - DNA, 28 bp, Artificial Sequence
  Synthetic
actcaaattcatcccagccgtccagctg

SEQ ID NO: 99 - DNA, 31 bp, Artificial Sequence
  Synthetic
ttgtttatggatgcgctggggttgtcgatgg

SEQ ID NO: 100 - DNA, 48 bp, Artificial Sequence
  Synthetic
ctgggcaatttctgccatattcataagcttctttctcctctttcctcg

SEQ ID NO: 101 - DNA, 20 bp, Artificial Sequence
  Synthetic
gcagacgaacatttcgactg

SEQ ID NO: 102 - DNA, 20 bp, Artificial Sequence
  Synthetic
atcgccattgaatcctgtgc

SEQ ID NO: 103 - DNA, 52 bp, Artificial Sequence
  Synthetic
actgtagcgaaagcaaaagcttaataacgctgatagtgctagtgtagatcgc

SEQ ID NO: 104 - DNA, 50 bp, Artificial Sequence
  Synthetic
aggtcgataccaattattttacccattgagacctttctcctctttcctcg

SEQ ID NO: 105 - DNA, 46 bp, Artificial Sequence
  Synthetic
gaaagaggagaaaggtctcaatgggtaaaataattggtatcgacct

SEQ ID NO: 106 - DNA, 44 bp, Artificial Sequence
  Synthetic
cactagcactatcagcgttattaagcttttgctttcgctacagt

SEQ ID NO: 107 - DNA, 49 bp, Artificial Sequence
  Synthetic
ctgatgaaccagcaggcgtaataacgctgatagtgctagtgtagatcgc

SEQ ID NO: 108 - DNA, 49 bp, Artificial Sequence
  Synthetic
cgatcatgcaatggacgaatattcattgagacctttctcctctttcctc

SEQ ID NO: 109 - DNA, 46 bp, Artificial Sequence
  Synthetic
gaaagaggagaaaggtctcaatgaatattcgtccattgcatgatcg

SEQ ID NO: 110 - DNA, 41 bp, Artificial Sequence
  Synthetic
cactagcactatcagcgttattacgcctgctggttcatcag

SEQ ID NO: 111 - DNA, 1521 bp, Artificial Sequence
  Synthetic
atgaatatggcagaaattgcccagctgggtgtgagtaatccgtataaacagcagtatgaaaattatattggtggtgcatgggttccgccagctggcggtgaatattttgaatcaaccaccccgattaccggcaaaccgtttacccgtgttccgcgtagcggtcagcaggatgtggatgccgcactggatgcagcacatgcagccaaagccgcatgggcacgtacctctaccaccgaacgtgccaatattctgaatcgcattgccgatcgcattgaagccaatctgaaactgctggcagttgccgaatctattgataatggtaaaccggttcgtgaaaccaccgccgccgatctgccgttagcagtggatcattttcgctattttgcaggttgtattcgcgcccaggaaggcggcattagcgaaattgatgcagataccattgcatatcattttcatgaaccgttaggcgttgtgggccagattattccgtggaattttccgctgttaatggcaacctggaaactggccccggccttagcagcaggtaattgtgttgtgctgaaacccgccgaacagaccccggcctcaattctggtgttaatggaagtgattggcgatttactgccgccgggcgttgttaatgtgattaatggctttggcttagaagcaggtaaaccgctggcaagctctccgcgcatttctaaagttgcctttaccggcgaaaccaccaccggtcgtctgattatgcagtatgcaagtcagaatctgattccggtgaccttagaactgggtggtaaaagtccgaatattttttttgaagatgtgctggccgccgatgatgccttttttgataaagccctggaaggctttgccatgtttgcactgaatcagggcgaagtttgtacctgtccgtcacgcgcactgattcaggaatcaatttatgatcgctttatggaacgcgccttaaaacgggttgcagcaattcgtcagggccatccgttagataccggtaccatgattggcgcacaggcctctgccgaacagttagaaaaaattctgagctatattgatctgggtcgcaaagaaggcgcccagtgtctgaccggcggtgaacgtaatgtgctggatggcgatttagccggtggctattatgttaaaccgaccgtgtttgcaggtcataataaaatgcgcatttttcaggaagaaatttttggtccggttgtgagcgtgaccacctttaaagatgaagaagaagccttagccattgccaatgataccctgtatggtttaggtgcaggcgtgtggacccgcgatggtgcacgcgcctttcgtatgggtcgtggtattcaggcaggtcgcgtttggaccaattgttatcatgcctatccggcacatgcagcctttggcggctataaacagagcggtattggtcgcgaaaatcatcgtatgatgttagatcattatcagcagaccaaaaatctgttagtgtcttatagtccgaatgccctgggctttttttaa

SEQ ID NO: 112 - DNA, 1771 bp, Artificial Sequence
  Synthetic
ctagagtctcgagttgacagctagctcagtcctaggtataatgctagcctcgaggaaagaggagaaagaagcttatgaccaataatcccccttcagcacagattaagcccggcgagtatggtttccccctcaagttaaaagcccgctatgacaactttattggcggcgaatgggtagcccctgccgacggcgagtattaccagaatctgacgccggtgaccgggcagctgctgtgcgaagtggcgtcttcgggcaaacgagacatcgatctggcgctggatgctgcgcacaaagtgaaagataaatgggcgcacacctcggtgcaggatcgtgcggcgattctgtttaagattgccgatcgaatggaacaaaacctcgagctgttagcgacagctgaaacctgggataacggcaaacccattcgcgaaaccagtgctgcggatgtaccgctggcgattgaccatttccgctatttcgcctcgtgtattcgggcgcaggaaggtgggatcagtgaagttgatagcgaaaccgtggcctatcatttccatgaaccgttaggcgtggtggggcagattatcccgtggaacttcccgctgctgatggcgagctggaaaatggctcccgcgctggcggcgggcaactgtgtggtgctgaaacccgcacgtcttaccccgctttctgtactgctgctaatggaaattgtcggtgatttactgccgccgggcgtggtgaacgtggtcaatggcgcaggtggggtaattggcgaatatctggcgacctcgaaacgcatcgccaaagtggcgtttaccggctcaacggaagtgggccaacaaattatgcaatacgcaacgcaaaacattattccggtgacgctggagttgggcggtaagtcgccaaatatcttctttgctgatgtgatggatgaagaagatgcctttttcgataaagcgctggaaggctttgcactgtttgcctttaaccagggcgaagtttgcacctgtccgagtcgtgctttagtgcaggaatctatctacgaacgctttatggaacgcgccatccgccgtgtcgaaagcattcgtagcggtaacccgctcgacagcgtgacgcaaatgggcgcgcaggtttctcacgggcaactggaaaccatcctcaactacattgatatcggtaaaaaagagggcgctgacgtgctcacaggcgggcggcgcaagctgctggaaggtgaactgaaagacggctactacctcgaaccgacgattctgtttggtcagaacaatatgcgggtgttccaggaggagatttttggcccggtgctggcggtgaccaccttcaaaacgatggaagaagcgctggagctggcgaacgatacgcaatatggcctgggcgcgggcgtctggagccgcaacggtaatctggcctataagatggggcgcggcatacaggctgggcgcgtgtggaccaactgttatcacgcttacccggcacatgcggcgtttggtggctacaaacaatcaggtatcggtcgcgaaacccacaagatgatgctggagcattaccagcaaaccaagtgcctgctggtgagctactcggataaaccgttggggctgttctgagagctcgatagtgctagtgtagatcgctactagagccaggcatcaaataaaacgaaaggctcagtcgaaagactgggcctttcgttttatctgttgtttgtcggtgaacgctctctactagagtcacactggctcaccttcgggtgggcctttctgcg

SEQ ID NO: 113 - DNA, 1806 bp, Artificial Sequence
  Synthetic
ctagagtctcgagttgacagctagctcagtcctaggtataatgctagcctcgaggaaagaggagaaagaagcttatgaaagcaattttcgtactgaaacatcttaatcatgctaaggaggttttctaatgaatatggcagaaattgcccagctgggtgtgagtaatccgtataaacagcagtatgaaaattatattggtggtgcatgggttccgccagctggcggtgaatattttgaatcaaccaccccgattaccggcaaaccgtttacccgtgttccgcgtagcggtcagcaggatgtggatgccgcactggatgcagcacatgcagccaaagccgcatgggcacgtacctctaccaccgaacgtgccaatattctgaatcgcattgccgatcgcattgaagccaatctgaaactgctggcagttgccgaatctattgataatggtaaaccggttcgtgaaaccaccgccgccgatctgccgttagcagtggatcattttcgctattttgcaggttgtattcgcgcccaggaaggcggcattagcgaaattgatgcagataccattgcatatcattttcatgaaccgttaggcgttgtgggccagattattccgtggaattttccgctgttaatggcaacctggaaactggccccggccttagcagcaggtaattgtgttgtgctgaaacccgccgaacagaccccggcctcaattctggtgttaatggaagtgattggcgatttactgccgccgggcgttgttaatgtgattaatggctttggcttagaagcaggtaaaccgctggcaagctctccgcgcatttctaaagttgcctttaccggcgaaaccaccaccggtcgtctgattatgcagtatgcaagtcagaatctgattccggtgaccttagaactgggtggtaaaagtccgaatattttttttgaagatgtgctggccgccgatgatgccttttttgataaagccctggaaggctttgccatgtttgcactgaatcagggcgaagtttgtacctgtccgtcacgcgcactgattcaggaatcaatttatgatcgctttatggaacgcgccttaaaacgggttgcagcaattcgtcagggccatccgttagataccggtaccatgattggcgcacaggcctctgccgaacagttagaaaaaattctgagctatattgatctgggtcgcaaagaaggcgcccagtgtctgaccggcggtgaacgtaatgtgctggatggcgatttagccggtggctattatgttaaaccgaccgtgtttgcaggtcataataaaatgcgcatttttcaggaagaaatttttggtccggttgtgagcgtgaccacctttaaagatgaagaagaagccttagccattgccaatgataccctgtatggtttaggtgcaggcgtgtggacccgcgatggtgcacgcgcctttcgtatgggtcgtggtattcaggcaggtcgcgtttggaccaattgttatcatgcctatccggcacatgcagcctttggcggctataaacagagcggtattggtcgcgaaaatcatcgtatgatgttagatcattatcagcagaccaaaaatctgttagtgtcttatagtccgaatgccctgggctttttttaagagctcgatagtgctagtgtagatcgctactagagccaggcatcaaataaaacgaaaggctcagtcgaaagactgggcctttcgttttatctgttgtttgtcggtgaacgctctctactagagtcacactggctcaccttcgggtgggcctttctgcg

SEQ ID NO: 114 - DNA, 2506 bp, Artificial Sequence
  Synthetic
ttgtttatggatgcgctggggttgtcgatggcgctcggtacgtttattgcgggtgtgctactggcggaaagtgaatatcgccatgaactggaaacggctatcgatcccttcaaaggcttgctgctcggtttgttctttatctctgtcggcatgtcgctcaacctcggggtgctttatacccatctgttgtgggtagtgataagtgtggttgtgctggtggcggtgaaaattctcgtgctgtatctgctggcgcgattgtatggcgtgcgcagttctgagcggatgcagtttgctggcgtgttgagtcagggcggtgagtttgcctttgtcctcttttctaccgcttcttcacaacgcttattccagggcgaccagattctagagtctcgagttgacagctagctcagtcctaggtataatgctagcctcgaggaaagaggagaaagaagcttatgaccaataatcccccttcagcacagattaagcccggcgagtatggtttccccctcaagttaaaagcccgctatgacaactttattggcggcgaatgggtagcccctgccgacggcgagtattaccagaatctgacgccggtgaccgggcagctgctgtgcgaagtggcgtcttcgggcaaacgagacatcgatctggcgctggatgctgcgcacaaagtgaaagataaatgggcgcacacctcggtgcaggatcgtgcggcgattctgtttaagattgccgatcgaatggaacaaaacctcgagctgttagcgacagctgaaacctgggataacggcaaacccattcgcgaaaccagtgctgcggatgtaccgctggcgattgaccatttccgctatttcgcctcgtgtattcgggcgcaggaaggtgggatcagtgaagttgatagcgaaaccgtggcctatcatttccatgaaccgttaggcgtggtggggcagattatcccgtggaacttcccgctgctgatggcgagctggaaaatggctcccgcgctggcggcgggcaactgtgtggtgctgaaacccgcacgtcttaccccgctttctgtactgctgctaatggaaattgtcggtgatttactgccgccgggcgtggtgaacgtggtcaatggcgcaggtggggtaattggcgaatatctggcgacctcgaaacgcatcgccaaagtggcgtttaccggctcaacggaagtgggccaacaaattatgcaatacgcaacgcaaaacattattccggtgacgctggagttgggcggtaagtcgccaaatatcttctttgctgatgtgatggatgaagaagatgcctttttcgataaagcgctggaaggctttgcactgtttgcctttaaccagggcgaagtttgcacctgtccgagtcgtgctttagtgcaggaatctatctacgaacgctttatggaacgcgccatccgccgtgtcgaaagcattcgtagcggtaacccgctcgacagcgtgacgcaaatgggcgcgcaggtttctcacgggcaactggaaaccatcctcaactacattgatatcggtaaaaaagagggcgctgacgtgctcacaggcgggcggcgcaagctgctggaaggtgaactgaaagacggctactacctcgaaccgacgattctgtttggtcagaacaatatgcgggtgttccaggaggagatttttggcccggtgctggcggtgaccaccttcaaaacgatggaagaagcgctggagctggcgaacgatacgcaatatggcctgggcgcgggcgtctggagccgcaacggtaatctggcctataagatggggcgcggcatacaggctgggcgcgtgtggaccaactgttatcacgcttacccggcacatgcggcgtttggtggctacaaacaatcaggtatcggtcgcgaaacccacaagatgatgctggagcattaccagcaaaccaagtgcctgctggtgagctactcggataaaccgttggggctgttctgagagctcgatagtgctagtgtagatcgctactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagaagcggccgctgcagttgcatattcttgcgcgagcgcgcggacgtgtggaagcgcatgagttattacaggcaggggtgacgcagttttcccgtgaaacattctccagtgcgttagagctggggcgcaagacgctggtcacgcttggcatgcatccgcatcaggcgcagcgcgcgcaactgcattttcgccgcctggatatgcgaatgctgcgggagttaatcccgatgcatgctgataccgtacaaatttctcgcgccagggaagcccgacgtgaactggaagagattttccagcgtgaaatgcaacaagaacgacgccagctggacggctgggatgaatttgag

SEQ ID NO: 115 - DNA, 2542 bp, Artificial Sequence
  Synthetic
ttgtttatggatgcgctggggttgtcgatggcgctcggtacgtttattgcgggtgtgctactggcggaaagtgaatatcgccatgaactggaaacggctatcgatcccttcaaaggcttgctgctcggtttgttctttatctctgtcggcatgtcgctcaacctcggggtgctttatacccatctgttgtgggtagtgataagtgtggttgtgctggtggcggtgaaaattctcgtgctgtatctgctggcgcgattgtatggcgtgcgcagttctgagcggatgcagtttgctggcgtgttgagtcagggcggtgagtttgcctttgtcctcttttctaccgcttcttcacaacgcttattccagggcgaccagattctagagtctcgagttgacagctagctcagtcctaggtataatgctagcctcgaggaaagaggagaaagaagcttatgaaagcaattttcgtactgaaacatcttaatcatgctaaggaggttttctaatgaatatggcagaaattgcccagctgggtgtgagtaatccgtataaacagcagtatgaaaattatattggtggtgcatgggttccgccagctggcggtgaatattttgaatcaaccaccccgattaccggcaaaccgtttacccgtgttccgcgtagcggtcagcaggatgtggatgccgcactggatgcagcacatgcagccaaagccgcatgggcacgtacctctaccaccgaacgtgccaatattctgaatcgcattgccgatcgcattgaagccaatctgaaactgctggcagttgccgaatctattgataatggtaaaccggttcgtgaaaccaccgccgccgatctgccgttagcagtggatcattttcgctattttgcaggttgtattcgcgcccaggaaggcggcattagcgaaattgatgcagataccattgcatatcattttcatgaaccgttaggcgttgtgggccagattattccgtggaattttccgctgttaatggcaacctggaaactggccccggccttagcagcaggtaattgtgttgtgctgaaacccgccgaacagaccccggcctcaattctggtgttaatggaagtgattggcgatttactgccgccgggcgttgttaatgtgattaatggctttggcttagaagcaggtaaaccgctggcaagctctccgcgcatttctaaagttgcctttaccggcgaaaccaccaccggtcgtctgattatgcagtatgcaagtcagaatctgattccggtgaccttagaactgggtggtaaaagtccgaatattttttttgaagatgtgctggccgccgatgatgccttttttgataaagccctggaaggctttgccatgtttgcactgaatcagggcgaagtttgtacctgtccgtcacgcgcactgattcaggaatcaatttatgatcgctttatggaacgcgccttaaaacgggttgcagcaattcgtcagggccatccgttagataccggtaccatgattggcgcacaggcctctgccgaacagttagaaaaaattctgagctatattgatctgggtcgcaaagaaggcgcccagtgtctgaccggcggtgaacgtaatgtgctggatggcgatttagccggtggctattatgttaaaccgaccgtgtttgcaggtcataataaaatgcgcatttttcaggaagaaatttttggtccggttgtgagcgtgaccacctttaaagatgaagaagaagccttagccattgccaatgataccctgtatggtttaggtgcaggcgtgtggacccgcgatggtgcacgcgcctttcgtatgggtcgtggtattcaggcaggtcgcgtttggaccaattgttatcatgcctatccggcacatgcagcctttggcggctataaacagagcggtattggtcgcgaaaatcatcgtatgatgttagatcattatcagcagaccaaaaatctgttagtgtcttatagtccgaatgccctgggctttttttaagagctcgatagtgctagtgtagatcgctactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagaagcggccgctgcagttgcatattcttgcgcgagcgcgcggacgtgtggaagcgcatgagttattacaggcaggggtgacgcagttttcccgtgaaacattctccagtgcgttagagctggggcgcaagacgctggtcacgcttggcatgcatccgcatcaggcgcagcgcgcgcaactgcattttcgccgcctggatatgcgaatgctgcgggagttaatcccgatgcatgctgataccgtacaaatttctcgcgccagggaagcccgacgtgaactggaagagattttccagcgtgaaatgcaacaagaacgacgccagctggacggctgggatgaatttgagt

(19) United States
(12) Patent Application Publication
(10) Pub. No.: US 2024/0122993 A1
(43) Pub. Date: Apr. 18, 2024

(54) GENETICALLY GENGINEERED BACTERIUM FOR HANGOVER AND LIVER DISEASE PREVENTION AND/OR TREATMENT

(71) Applicant: COMMBIO THERAPEUTICS CO., LTD., Shanghai (CN)

(72) Inventors: Bin Xiang, Shanghai (CN); Shengming Yin, Shanghai (CN); Yanning Wang, Shanghai (CN)

(21) Appl. No.: 18/276,119

(22) PCT Filed: Feb. 8, 2022
(86) PCT No.: PCT/CN2022/075470

§ 371 Date: Aug. 7, 2023

(30) Foreign Application Priority Data
Feb. 8, 2021 (WO) PCT/CN2021/076102

(51) Int. Cl. A61K 35/741; A23L 33/135; C12N 9/02

(52) U.S. Cl. CPC A61K 35/741; A23L 33/135; C12N 9/0008; C12Y 102/0101; A61K 2035/115

(57) ABSTRACT

The present disclosure provides a genetically engineered probiotic, including an exogenous expression cassette including a nucleotide sequence that encodes acetaldehyde dehydrogenase, wherein the probiotic intestinal bacterium is Escherichia coli strain Nissle 1917 (EcN), and uses thereof.

### CROSS REFERENCE TO RELATED APPLICTIONS

This is a U.S. National Phase application based upon PCT Application No. PCT/CN2022/075470, filed Feb. 8, 2022 and titled “GENETICALLY GENGINEERED BACTERIUM FOR HANGOVER AND LIVER DISEASE PREVENTION AND/OR TREATMENT,” which claims priority to PCT Application No. PCT/CN2021/076102, filed Feb. 8, 2021, the disclosures of which are hereby incorporated by reference in their entirety.

### FIELD OF THE INVENTION

The present disclosure generally relates to the fields of genetically engineered probiotic intestinal bacterium, and its application in preventing and/or treating hangover and liver diseases.

### BACKGROUND

Hangovers represent a major problem and a huge source of economic loss to society. Hangovers and their associated problems (e.g., alcoholic liver diseases) have been recognized for thousands of years in both Western and Eastern cultures. However, few effective prevention and/or treatment methods for hangovers and associated liver problems are available.

Therefore, there is need for developing novel methods for preventing and/or treating hangover and alcoholic liver diseases.

### SUMMARY OF THE INVENTION

In one aspect, the present disclosure provides a genetically engineered probiotic intestinal bacterium comprising an exogenous expression cassette comprising a nucleotide sequence that encodes acetaldehyde dehydrogenase, wherein the probiotic intestinal bacterium is Escherichia coli strain Nissle 1917 (EcN).

In some embodiments, the acetaldehyde dehydrogenase is a naturally-occurring AcoD from Cupriavidus necator, or a functional equivalent thereof.

In some embodiments, the functional equivalent retains at least partial activity in oxidizing aldehydes.

In some embodiments, the functional equivalent comprises a mutant, a fragment, a fusion, a derivative, or any combination thereof of the naturally-occurring AcoD.

In some embodiments, the acetaldehyde dehydrogenase comprises an amino acid sequence of SEQ ID NO: 1, or an amino acid sequence having at least 80% sequence identity thereof yet retaining substantial activity in oxidizing aldehydes.

In some embodiments, the nucleotide sequence that encodes acetaldehyde dehydrogenase has been codon-optimized for expression in EcN, and optionally, the codon-optimized nucleotide sequence comprises a sequence of SEQ ID NO: 111 or a homologous sequence thereof having at least 80% sequence identity.

In some embodiments, the expression cassette further comprises one or more regulatory elements comprising one or more elements selected from the group consisting of: a promoter, a ribosome binding site (RBS), a terminator, cistron and any combination thereof.

In some embodiments, the promoter is a constitutive promoter, or an inducible promoter.

In some embodiments, the promoter is an endogenous promoter, or an exogenous promoter.

In some embodiments, the constitutive promoter comprises a nucleotide sequence selected from the group consisting of SEQ ID NOs: 10-49 and homologous sequences thereof having at least 80% sequence identity.

In some embodiments, the constitutive promoter comprises a nucleic acid sequence of SEQ ID NO: 10.

In some embodiments, the inducible promoter comprises an anaerobic inducible promoter.

In some embodiments, the anaerobic inducible promoter comprises a nucleotide sequence of SEQ ID NO: 53.

In some embodiments, the RBS comprises a nucleotide sequence selected from the group consisting of SEQ ID NOs: 65-67 and homologous sequences thereof having at least 80% sequence identity.

In some embodiments, the terminator is T7 terminator.

In some embodiments, the cistron is BCD2.

In some embodiments, the cistron comprises a nucleotide sequence of SEQ ID NO: 62 or homologous sequences thereof having at least 80% sequence identity.

In some embodiments, the exogenous expression cassette is integrated in the genome of the genetically engineered probiotic intestinal bacterium.

In some embodiments, the genetically engineered probiotic intestinal bacterium expresses at least one nucleotide sequence that encodes at least one Chaperone protein selected from the group consisting of: dsbA, dsbC, dnaK, dnaJ, grpE, groES, groEL, tig, fkpA, surA, skp, PpiD and DegP.

In some embodiments, the genetically engineered probiotic intestinal bacterium further comprises at least one inactivation or deletion in an auxotroph-related gene.

In some embodiments, the probiotic intestinal bacterium is an auxotroph for one or more substances selected from the group consisting of thymidine, uracil, leucine, histidine, tryptophan, lysine, methionine, adenine, and non-naturally occurring amino acid.

In one aspect, the present disclosure provides a recombinant expression cassette comprising a nucleotide sequence that encodes AcoD, and one or more regulatory elements, wherein the nucleotide sequence has been optimized for expression in EcN, and optionally, the codon-optimized nucleotide sequence comprises a sequence of SEQ ID NO: 111 or a homologous sequence thereof having at least 80% sequence identity.

In some embodiments, the recombinant expression cassette further comprises one or more regulatory elements selected from the group consisting of: a promoter, a ribosome binding site (RBS), a terminator, cistron and any combination thereof.

In some embodiments, the promoter is a constitutive promoter, or an inducible promoter (e.g., an anaerobic inducible promoter).

In some embodiments, the promoter is an endogenous promoter, or an exogenous promoter.

In some embodiments, the promoter comprises a nucleotide sequence selected from the group consisting of SEQ ID NOs: 10-53 and homologous sequences thereof having at least 80% sequence identity.

In some embodiments, the promoter comprises a nucleotide sequence of SEQ ID NO: 10.

In some embodiments, the RBS comprises a nucleotide sequence selected from the group consisting of SEQ ID NOs: 65-67 and homologous sequences thereof having at least 80% sequence identity.

In some embodiments, the terminator is T7 terminator.

In some embodiments, the cistron is BCD2.

In some embodiments, the cistron comprises a nucleotide sequence of SEQ ID NO: 62 or homologous sequences thereof having at least 80% sequence identity.

In one aspect, the present disclosure provides a composition comprising the genetically engineered probiotic intestinal bacterium provided herein, and a physiologically acceptable carrier.

In some embodiments, the composition is edible.

In some embodiments, the composition is a food supplement.

In some embodiments, the composition further comprises one or more physiologically acceptable carrier selected from lactic acid fermented foods, fermented dairy products, resistant starch, dietary fibers, carbohydrates, fat, oil, flavoring agent, seasoning agent, proteins and glycosylated proteins, water, capsule filler, and a gummy material.

In some embodiments, the genetically-engineered microorganism is a live cell.

In some embodiments, the composition is a finished food product, a powder, a granule, a tablet, a capsule, or a liquid.

In some embodiments, the composition comprises about 0.01 to about 99.9% by weight genetically-engineered microbe.

In one aspect, the present disclosure provides a method for preventing and/or treating an alcohol hangover in a subject in need thereof, comprising administering to the gut of the subject an effective amount of the genetically engineered probiotic intestinal bacterium or the composition provided herein.

In one aspect, the present disclosure provides a method for reducing levels of acetaldehyde in a subject in need thereof, comprising administering to the gut of the subject an effective amount of the genetically engineered probiotic intestinal bacterium or the composition provided herein.

In one aspect, the present disclosure provides a method for preventing and/or treating Asian flush in a subject in need thereof, comprising administering to the gut of the subject an effective amount of the genetically engineered probiotic intestinal bacterium or the composition provided herein.

In some embodiments, the subject is deficient in one or more alcohol dehydrogenases.

In some embodiments, the subject is deficient in one or more aldehyde dehydrogenases.

In some embodiments, the composition is administered before, during, or after consumption of alcohol.

In some embodiments, the method comprises administering the composition to the subject up to 24 hours before commencement of consumption of alcohol.

In some embodiments, the subject is a carrier of ALDH2 variant alleles.

In one aspect, the present disclosure provides a method for preventing and/or treating alcoholic liver disease in a subject in need thereof, comprising administering to the gut of the subject an effective amount of the genetically engineered probiotic intestinal bacterium or the composition provided herein.

In some embodiments, the alcoholic liver disease is alcoholic fatty liver, alcoholic hepatitis or alcoholic liver cirrhosis.

In one aspect, the present disclosure provides a method for preventing and/or slowing down progression of alcoholic fatty liver disease into alcoholic liver fibrosis, alcoholic liver cirrhosis or alcoholic liver cancer in a subject in need thereof, comprising administering to the gut of the subject an effective amount of the genetically engineered probiotic intestinal bacterium or the composition provided herein.

In one aspect, the present disclosure provides a method for preventing and/or slowing down progression of alcoholic hepatitis into alcoholic liver fibrosis, alcoholic liver cirrhosis or alcoholic liver cancer in a subject in need thereof, comprising administering to the gut of the subject an effective amount of the genetically engineered probiotic intestinal bacterium or the composition provided herein.

In one aspect, the present disclosure provides a method for preventing and/or treating non-alcoholic fatty liver (NAFLD) or non-alcoholic steatohepatitis (NASH) in a subject in need thereof, comprising administering to the gut of the subject an effective amount of the genetically engineered probiotic intestinal bacterium or the composition provided herein.

In one aspect, the present disclosure provides a method for preventing and/or slowing progression of NAFLD into NASH in a subject in need thereof, comprising administering to the gut of the subject an effective amount of the genetically engineered probiotic intestinal bacterium or the composition provided herein.

In one aspect, the present disclosure provides a method for preventing and/or slowing progression of NASH into liver fibrosis in a subject in need thereof, comprising administering to the gut of the subject an effective amount of the genetically engineered probiotic intestinal bacterium or the composition provided herein.

In certain embodiments, the subject has an elevated level of blood ethanol and/or increased abundance of alcohol-producing gut microbiota.

### BRIEF DESCFRIPTION OF THE DRAWINGS

FIG. 1 shows the plasmid profile of gRNA plasmid ZL-003_kefB.

FIG. 2 shows the PCR electropherogram of kefB-J23119-AcoD, kefB-J23101-AcoD and kefB-J23108-AcoD.

FIG. 3A shows the tolerance test result of the Control Bacteria, Engineered Bacteria 119 and Engineered Bacteria 101.

FIG. 3B shows the relative expression level of AcoD in Engineered Bacteria 119, Engineered Bacteria 101 and Engineered Bacteria 108.

FIG. 4 shows the capability of acetaldehyde removal of the Control Bacteria, Engineered Bacteria 119, Engineered Bacteria 101 and Engineered Bacteria 108 in vitro.

FIG. 5 shows the capability of acetaldehyde removal of the Control Bacteria and Engineered Bacteria 119 in vivo.

FIG. 6 shows the capability of acetaldehyde removal of the Control Bacteria, Engineered Bacteria 119, Engineered Bacteria 119 expressing Gro and Engineered Bacteria 119 expressing KJE in vitro.

FIG. 7 shows the capability of in vitro acetaldehyde removal of the Control Bacteria (EcN), Engineered Bacteria 119-AcoD (119-AcoD), Engineered Bacteria AldB overexpressing E. coli endogenous 119-AldB gene by J23119 promoter (119-aldB).

FIG. 8 shows the capability of in vitro acetaldehyde removal of the Control Bacteria (EcN), Engineered Bacteria AcoD (AcoD), and Engineered Bacteria BCD2 with BCD2 cistron added upstream the open reading frame of AcoD (BCD2).

### DETAILED DESCRIPTION OF THE INVENTION

Throughout the present disclosure, the articles “a,” “an,” and “the” are used herein to refer to one or to more than one (i.e., to at least one) of the grammatical object of the article. By way of example, “an antibody” means one antibody or more than one antibody.

The following description of the disclosure is merely intended to illustrate various embodiments of the disclosure. As such, the specific modifications discussed are not to be construed as limitations on the scope of the disclosure. It will be apparent to a person skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entireties.

### I. Definitions

The term “effective amount” or “pharmaceutically effective amount” as used herein refers to the amount and/or dosage, and/or dosage regime of one or more agents necessary to bring about the desired results, e.g., an amount sufficient to mitigate in a subject one or more symptoms associated with a condition or a disease for which the subject is receiving a therapy or a composition, or an amount sufficient to lessen the severity or delay the progression of the condition in a subject (e.g., therapeutically effective amounts), an amount sufficient to reduce the risk or delaying the onset, and/or reduce the ultimate severity of a disease or condition in a subject (e.g., prophylactically effective amounts).

The term “encodes”, “encoded” or “encoding” as used herein means capable of transcription into mRNA and/or translation into a peptide or protein. The term “encoding sequence” or “gene” refers to a polynucleotide sequence encoding a peptide or protein. These two terms can be used interchangeably in the present disclosure. In some embodiments, the encoding sequence is a complementary DNA (cDNA) sequence that is reversely transcribed from a messenger RNA (mRNA). In some embodiments, the encoding sequence is mRNA.

The term “homologous” as used herein refers to nucleic acid sequences (or its complementary strand) or amino acid sequences that have sequence identity of at least 60% (e.g. at least 65%, 70%, 75%, 80%, 85%, 88%, 90%, 91%, 92%, 93%, 94%, 95%, 96%, 97%, 98%, 99%) to another sequences when optimally aligned.

The term “nucleotide sequence”, “nucleic acid” or “polynucleotide” as used herein includes oligonucleotides (i.e., short polynucleotides). They also refer to synthetic and/or non-naturally occurring nucleic acid molecules (e.g., comprising nucleotide analogues or modified backbone residues or linkages). The terms also refer to deoxyribonucleotide or ribonucleotide oligonucleotides in either single-or double-stranded form. The terms encompass nucleic acids containing analogues of natural nucleotides. The terms also encompass nucleic acid-like structures with synthetic backbones. Unless otherwise indicated, a particular polynucleotide sequence also implicitly encompasses conservatively modified variants thereof (e.g. degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). In other words, percent (%) sequence identity of an amino acid sequence (or nucleic acid sequence) can be calculated by dividing the number of amino acid residues (or bases) that are identical relative to the reference sequence to which it is being compared by the total number of the amino acid residues (or bases) in the candidate sequence or in the reference sequence, whichever is shorter. Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al., J. Mol. Biol., 215:403-410 (1990); Stephen F. et al., Nucleic Acids Res., 25:3389-3402 (1997)), ClustalW2 (available on the website of European Bioinformatics Institute, see also, Higgins D. G. et al., Methods in Enzymology, 266:383-402 (1996); Larkin M. A. et al., Bioinformatics (Oxford, England), 23(21):2947-8 (2007)), and ALIGN or Megalign (DNASTAR) software. A person skilled in the art may use the default parameters provided by the tool, or may customize the parameters as appropriate for the alignment, such as for example, by selecting a suitable algorithm.

The term “probiotic” as used herein means non-pathogenic. In some embodiments, a probiotic microbial cell, when administered in an effective amount, provide a beneficial effect on the health or well-being of a subject, including, for example, a health benefit that is associated with improving the balance of human or animal microbiota, and/or for restoring a normal microbiota. The term “probiotics” as used herein refers to preparations of probiotic microbial cell (such as, living microbial cells).

The term “subject” as used herein includes human and non-human animals. Non-human animals include all vertebrates, e.g., mammals and non-mammals, such as non-human primates, mice, rats, cats, rabbits, sheep, dogs, cows, chickens, amphibians, and reptiles. Except when noted, the terms “patient” or “subject” are used herein interchangeably.

“Treating” or “treatment” of a disease, disorder or condition as used herein includes preventing or alleviating a disease, disorder or condition, slowing the onset or rate of development of a disease, disorder or condition, reducing the risk of developing a disease, disorder or condition, preventing or delaying the development of symptoms associated with a disease, disorder or condition, reducing or ending symptoms associated with a disease, disorder or condition, generating a complete or partial regression of a disease, disorder or condition, curing a disease, disorder or condition, or some combination thereof.

The term “naturally-occurring” as used herein with respect to AcoD, means that the sequence of AcoD polypeptide or polynucleotide is identical to that or those found in nature. A naturally-occurring AcoD can be a native or wild-type sequence of AcoD, or a fragment thereof, even if the fragment itself may not be found in nature. A naturally-occurring AcoD can also include a naturally-occurring variant such as mutants or isoforms or different native sequences found in different bacteria strains. A naturally-occurring full-length AcoD polypeptide has a length of 506 amino acid residues. Exemplary amino acid sequences of naturally-occurring AcoD include, without limitation, AcoD (SEQ ID NO: 1).

### II. Genetically Engineered Probiotic Intestinal Bacteria and Recombinant Expression Cassette

### Genetically Engineered Probiotic Intestinal Bacteria

### Acetaldehyde Dehydrogenase AcoD

In one aspect, the present disclosure provides a genetically engineered probiotic intestinal bacterium comprising an exogenous expression cassette comprising a nucleotide sequence that encodes acetaldehyde dehydrogenase, wherein the probiotic intestinal bacterium is Escherichia coli strain Nissle 1917 (EcN).

As used herein, the term “acetaldehyde dehydrogenase” refers to an enzyme or a functional equivalent thereof that is capable of catalyzing oxidization of acetaldehyde into acetate. In certain embodiments, the acetaldehyde dehydrogenase is from human. In certain embodiments, the acetaldehyde dehydrogenase is from a non-human organism, e.g., Cupriavidus necator. In certain embodiments, the acetaldehyde dehydrogenase is acetaldehyde dehydrogenase2 (ALDH2). The term “ALDH2” can refer to protein of ALDH2 as well as the gene of ALDH2. In certain embodiments, the acetaldehyde dehydrogenase is AcoD. The term “functional equivalent” as used herein with respect to acetaldehyde dehydrogenase, ALDH2, or AcoD (e.g., from Cupriavidus necator) means any acetaldehyde dehydrogenase variant that, despite of having difference in amino acid sequences or polynucleotide sequences or in chemical structures, retains at least partially, one or more biological functions of acetaldehyde dehydrogenase, ALDH2, or AcoD (e.g., from Cupriavidus necator). The biological function of acetaldehyde dehydrogenase, ALDH2, or AcoD (e.g., from Cupriavidus necator) include, without limitation, catalyzing oxidation of acetaldehyde into acetate, ethanol degradation, ketone degradation.

In certain embodiments, the acetaldehyde dehydrogenase is a naturally-occurring AcoD from Cupriavidus necator, or a functional equivalent thereof. As used herein, the term “AcoD” refers to the protein of acetaldehyde dehydrogenase from Cupriavidus necator; as well as any and all genes encoding such an AcoD protein.

In certain embodiments, the functional equivalent of a naturally-occurring AcoD retains at least partial activity in oxidizing aldehydes. The functional equivalent can comprise a mutant, a fragment, a fusion, a derivative, or any combination thereof of the naturally-occurring AcoD. In certain embodiments, the AcoD comprises an amino acid sequence of SEQ ID NO: 1, or an amino acid sequence having at least 80% sequence identity thereof yet retaining substantial activity in oxidizing acetaldehydes.

In certain embodiments, the nucleotide sequence that encodes AcoD has been codon-optimized for expression in EcN, and optionally, the codon-optimized nucleotide sequence comprises a sequence of SEQ ID NO: 111 or a homologous sequence thereof having at least 80% sequence identity. The term “codon-optimized” as used herein refers to that the nucleotide sequence encoding a polypeptide has been configured to comprise codons preferred by the host cell or organism, e.g., EcN, for improved gene expression and increased translational efficiency in the host cell or organism.

### Regulatory Elements

In certain embodiments, the expression cassette further comprises one or more regulatory elements comprising one or more elements selected from the group consisting of: a promoter, a ribosome binding site (RBS), a terminator, and any combination thereof. The one or more regulatory elements are operably linked to the polynucleotide sequence of acetaldehyde dehydrogenase. The term “operably link” as used herein refers to a juxtaposition, with or without a spacer or linker, of two or more biological sequences of interest in such a way that they are in a relationship permitting them to function in an intended manner. The term may be used with respect to polynucleotides. For one instance, when a polynucleotide encoding a polypeptide is operably linked to a regulatory sequence (e.g., promoter, enhancer, silencer sequence, etc.), it is intended to mean that the polynucleotide sequences are linked in such a way that permits regulated expression of the polypeptide from the polynucleotide. When used with respect to polypeptides, it is intended to mean that the polypeptide sequences are linked in such a way that permits the linked product to have the intended biological function. For example, an antibody variable region may be operably linked to a constant region so as to provide for a stable product with antigen-binding activity.

### 1. Promoter

In certain embodiments, the promoter is a constitutive promoter, or an inducible promoter.

As used herein, the term “promoter” refers to a polynucleotide sequence that can control transcription of an encoding sequence. The promoter sequence includes specific sequences that are sufficient for RNA polymerase recognition, binding and transcription initiation. In addition, the promoter sequence may include sequences that modulate this recognition, binding and transcription initiation activity of RNA polymerases, optionally in the probiotic intestinal bacterium provided herein. The promoter may affect the transcription of a gene located on the same nucleic acid molecule as itself or a gene located on a different nucleic acid molecule as itself. Functions of the promoter sequences, depending upon the nature of the regulation, may be constitutive or inducible by a stimulus.

The term “constitutive promoter” refers to a promoter that is capable of facilitating continuous transcription of a coding sequence or gene under its control and/or to which it is operably linked. Constitutive promoters and variants for EcN are well known in the art and include, but are not limited to, BBa_J23119, BBa_J23101, BBa_J23102, BBa_J23103, BB a J23109, BBa_J23110, BBa_J23114, BBa_J23117, USP45_promoter, OmpA_promoter, BBa_J23100, BBa_J23104, BBa_J23105, BBa 114018, BBa_J45992, BBa_J23118, BBa_J23116, BBa_J23115, BBa_J23113, BBa_J23112, BBa_J23111, BBa_J23108, BBa_J23107, BBa_J23106, BBa_I14033, BBa_K256002, BBa_K1330002, BBa_J44002, BBa_J23150, BBa_I14034, Oxb19, oxb20, BBa_K088007, Ptet, Ptrc, PlacUV5, BBa_K292001, BBa_K292000, BBa_K137031, and BBa_K137029. The nucleotide sequences of exemplary constitutive promoters comprise a nucleotide sequence selected from the group consisting of SEQ ID NOs: 10-49 as shown in Table 1, and homologous sequences thereof having at least 80% (e.g. at least 85%, 90%, 91%, 92%, 93%, 94%, 95%, 96%, 97%, 98%, 99%) sequence identity. In some embodiments, the constitutive promoter comprises the nucleotide sequence of SEQ ID NO: 10. In some embodiments, such promoters are active in vitro, e.g., under culture, expansion and/or manufacture conditions. In some embodiments, such promoters are active in vivo, e.g., in conditions found in the in vivo environment, e.g., the gut microenvironment.

The term “inducible promoter” as used herein refers to a regulated promoter that can be turned on in one or more cell types by an external stimulus, such as a chemical, light, hormone, stress, anaerobic condition or a pathogen. Inducible promoters and variants are well known in the art and include, but are not limited to, PLteto1, galP1, PLlacO1, Pfnrs. In some embodiments, the nucleotide sequences of exemplary inducible promoters comprise a nucleotide sequence selected from the group consisting of SEQ ID NOs: 50-53 as shown in Table 1, and homologous sequences thereof having at least 80% (e.g. at least 85%, 90%, 91%, 92%, 93%, 94%, 95%, 96%, 97%, 98%, 99%) sequence identity. In certain embodiments, the inducible promoter comprises an anaerobic inducible promoter. In some embodiments, the inducible promoter comprises the nucleotide sequence of SEQ ID NO: 53.

In some embodiments, the promoter is an endogenous promoter, or an exogenous promoter. An “exogenous promoter” as used herein refers to a promoter in operable combination with a coding region wherein the promoter is not the promoter naturally associated with the coding region in the genome of an organism. The promoter which is naturally associated or linked to a coding region in the genome is referred to as the “endogenous promoter” for that coding region.

In certain embodiments, the constitutive promoter comprises a nucleotide sequence selected from the group consisting of SEQ ID NOs: 10-49 and homologous sequences thereof having at least 80% sequence identity. In certain embodiments, the constitutive promoter comprises SEQ ID NO: 10.

### 2. Ribosome Binding Site (RBS)

In certain embodiments, the RBS comprises a nucleotide sequence selected from the group consisting of SEQ ID NOs: 65-67 and homologous sequences thereof having at least 80% sequence identity. As used herein, the term “ribosome binding site” or “RBS” used interchangeably, refers to a sequence that the ribosome binds to when initiating protein translation. The RBS is approximately 35 nucleotides long and contains three discrete domains: (1) the Shine-Dalgarno (SD) sequence, (2) a spacer region, and (3) the first five to six codons of the Coding Sequence (CDS). RBSs and variants are well known in the art and include, but are not limited to, USP45, Synthesized, OmpA. The nucleotide sequences of exemplary RBSs comprise a nucleotide sequence selected from the group consisting of SEQ ID NOs: 65-67 as shown in Table 1, and homologous sequences thereof having at least 80% (e.g. at least 85%, 90%, 91%, 92%, 93%, 94%, 95%, 96%, 97%, 98%, 99%) sequence identity. In certain embodiments, the RBS comprises a nucleotide sequence selected from the group consisting of SEQ ID NOs: 66 and homologous sequences thereof having at least 80% sequence identity.

### 3. Terminator

In certain embodiments, the terminator is T7 terminator. The term “terminator” as used herein refers to an enzymatically incorporable nucleotide which prevents subsequent incorporation of nucleotides to the resulting polynucleotide chain and thereby halts polymerase-mediated extension. In some embodiments, the terminator comprises a nucleotide sequence selected from the group consisting of SEQ ID NOs: 68-69 as shown in Table 1 or a portion thereof, and homologous sequences thereof having at least 80% (e.g. at least 85%, 90%, 91%, 92%, 93%, 94%, 95%, 96%, 97%, 98%, 99%) sequence identity.

TABLE 1
The nucleotide sequences of exemplary promoters

| Regulatory | | | SEQ ID |
|---|---|---|---|
| element | Name | Nucleotide Sequence | NO. |
| Constitutive | BBa_J23119 | ttgacagctagctcagtcctaggtataatgctagc | 10 |
| promoter | BBa_J23101 | tttacagctagctcagtcctaggtattatgctagc | 11 |
| | BBa_J23102 | ttgacagctagctcagtcctaggtactgtgctagc | 12 |
| | BBa_J23103 | ctgatagctagctcagtcctagggattatgctagc | 13 |
| | BBa_J23109 | tttacagctagctcagtcctagggactgtgctagc | 14 |
| | BBa_J23110 | tttacggctagctcagtcctaggtacaatgctagc | 15 |
| | BBa_J23114 | tttatggctagctcagtcctaggtacaatgctagc | 16 |
| | BBa_J23117 | ttgacagctagctcagtcctagggattgtgctagc | 17 |
| | USP45_promoter | aaagtgttttgtaatcataaagaaatattaaggtggggtaggaatagt | 18 |
| | | ataatatgtttattcaaccgaacttaatg | |
| | OmpA_promoter | gtaaatttaggattaatcctggaactttttttgtcgcccagccaatgctt | 19 |
| | | tcagtcgtgactaattttccttgcggaggcttgtctgaagcggtttccg | |
| | | cgattttcttctgtaaattgtcgctgacaaaaaagattaaacgtacctt | |
| | | atacaagacttttttttcatatgcctgacggagttcacacttgtaagtttt | |
| | | caactacgttgtagactttacatcgccaggggtgctcggcataagcc | |
| | | gaagatatcggtagagttaatattgagcagatccccggtgaaggatt | |
| | | taaccgtgttatctcgttggagatattcatggcgtattttggatga | |
| | BBa_J23100 | ttgacggctagctcagtcctaggtacagtgctagc | 20 |
| | BBa_J23104 | ttgacagctagctcagtcctaggtattgtgctagc | 21 |
| | BBa_J23105 | tttacggctagctcagtcctaggtactatgctagc | 22 |
| | BBa_I14018 | gtttatacataggcgagtactctgttatgg | 23 |
| | BBa_J45992 | ggtttcaaaattgtgatctatatttaacaa | 24 |
| | BBa_J23118 | ttgacggctagctcagtcctaggtattgtgctagc | 25 |
| | BBa_J23116 | ttgacagctagctcagtcctagggactatgctagc | 26 |
| | BBa_J23115 | tttatagctagctcagcccttggtacaatgctagc | 27 |
| | BBa_J23113 | ctgatggctagctcagtcctagggattatgctagc | 28 |
| | BBa_J23112 | ctgatagctagctcagtcctagggattatgctagc | 29 |
| | BBa_J23111 | ttgacggctagctcagtcctaggtatagtgctagc | 30 |
| | BBa_J23108 | ctgacagctagctcagtcctaggtataatgctagc | 31 |
| | BBa_J23107 | tttacggctagctcagccctaggtattatgctagc | 32 |
| | BBa_J23106 | tttacggctagctcagtcctaggtatagtgctagc | 33 |
| | BBa_I14033 | agaggttccaactttcaccataatgaaaca | 34 |
| | BBa_K256002 | caccttcgggtgggcctttctgcgtttata | 35 |
| | BBa_K1330002 | ggctagctcagtcctaggtactatgctagc | 36 |
| | BBa_J44002 | aaagtgtgacgccgtgcaaataatcaatgt | 37 |
| | BBa_J23150 | ggctagctcagtcctaggtattatgctagc | 38 |
| | BBa_I14034 | taaacaactaacggacaattctacctaaca | 39 |
| | Oxb19 | aagctgttgtgaccgcttgctctagccagctatcgagttgtgaaccg | 40 |
| | | atccatctagcaattggtctcgatctagcgataggcttcgatctagct | |
| | | atgtatcactcattaggcaccccaggctttacactttatgcttccggct | |
| | | cgtataatgtgtggtgctggttagcgcttgctat | |
| | oxb20 | aagctgttgtgaccgcttgctctagccagctatcgagttgtgaaccg | 41 |
| | | atccatctagcaattggtctcgatctagcgataggcttcgatctagct | |
| | | atgtagaaacgccgtgtgctcgatcgcttgataaggtccacgtagct | |
| | | gctataattgcttcaacagaacatattgactatccggtattacccggc | |
| | BBa_K088007 | catacgccgttatacgttgtttacgctttg | 42 |
| | Ptet | taattcctaatttttgttgacactctatcgttgatagagttattttaccact | 43 |
| | | ccctatcagtgatagagaaaa | |
| | Ptrc | ttgacaattaatcatccggctcgtataatgtgtggaattgtgag | 44 |
| | PlacUV5 | cccaggctttacactttatgcttccggctcgtataatgtgtggaattgt | 45 |
| | | gag | |
| | BBa_K292001 | tgctagctactagagattaaagaggagaaa | 46 |
| | BBa_K292000 | ggctagctcagtcctaggtacagtgctagc | 47 |
| | BBa_K137031 | ccccgaaagcttaagaatataattgtaagc | 48 |
| | BBa_K137029 | atatatatatatatataatggaagcgtttt | 49 |
| Inducible | PLteto1 | tccctatcagtgatagagattgacatccctatcagtgatagagatact | 50 |
| promoter | | gagcacatcagcaggacgcactgacc | |
| | galP1 | attccactaatttattccatgtcacacttttcgcatctttgttatgctatgg | 51 |
| | | ttatttcataccataa | |
| | PLlacO1 | ataaatgtgagcggataacaattgacattgtgagcggataacaagat | 52 |
| | | actgagcacatcagcaggacgcactgacc | |
| | Pfnrs | aaaaacgccgcaaagtttgagcgaagtcaataaactctctacccatt | 53 |
| | | cagggcaatatctctctt | |
| Signal | Usp45 | atgaagaaaaagatcattagcgcgatcctgatgagcaccgtgattct | 54 |
| peptide | | gagcgcggcggcgccgctgagcggtgtttatgcg | |
| | OmpA | atgaagaaaaccgcgattgcgattgcggtggcgctggcgggtttc | 55 |
| | | gcgaccgttgcgcaggcg | |
| | DsbA | atgaagaaaatctggctggcgctggcgggtctggtgctggcgttca | 56 |
| | | gcgcgagcgcg | |
| | pelB | atgaagtacctgctgccgaccgcggcggcgggtctgctgctgctg | 57 |
| | | gcggcgcagccggcgatggcg | |
| | celCD | atggaaggaaacactcgtgaagacaattttaaacatttattaggtaat | 58 |
| | | gacaatgttaaacgc | |
| | sat | atgaataaaatatactcccttaaatatagtgctgccactggcggactc | 59 |
| | | attgctgtttctgaattagcgaaaagagtttctggtaaaacaaaccga | |
| | | aaacttgtagcaacaatgttgtctctggctgttgccggtacagtaaat | |
| | | gca | |
| | Endogenous | atgagcaactggatcaccgacaacaaaccggctgcgatggttgcg | 60 |
| | signal peptide | ggtgtgggcctgctgctgttcctgggtctgagcgcgaccggctac | |
| | of Amuc_1100 | | |
| Cistron | GFP | atgcgtaaaggcgaagagaaggaggttaactga | 61 |
| | BCD2 | atgaaagcaattttcgtactgaaacatcttaatcatgctaaggaggttt | 62 |
| | | tcta | |
| | luciferase | atgattatgtccggttataaggaggttaactga | 63 |
| | MBP | atgaaaatcgaagcaggtaaactggtacagaaggaggttaactga | 64 |
| RBS | USP45 | ggaggaaaaattaaaaaagaac | 65 |
| | Synthesized | aaagaggagaaa | 66 |
| | OmpA | taacgagg | 67 |
| Terminator | Terminator1 | cgagctcgatagtgctagtgtagatcgctactagagccaggcatca | 68 |
| | | aataaaacgaaaggctcagtcgaaagactgggcctttcgttttatct | |
| | | gttgtttgtcggtgaacgctctctactagagtcacactggctcaccttc | |
| | | gggtgggcctttctgcgtttatatactagaagcggccgctgcag | |
| | Terminator2 | cgagctcgatagtgctagtgtagatcgctactagagccaggcatca | 69 |
| | | aataaaacgaaagactgggcctttcgttttatctgttgtttgtcggtga | |
| | | acgctctctactagagtcacactggctcaccttcggggggcctttct | |
| | | gcgtttatatactagaagcggccgctgcag | |

### 4. Chaperon

In certain embodiments, the genetically engineered probiotic intestinal bacterium expresses at least one Chaperone protein selected from the group consisting of: dsbA, dsbC, dnaK, dnaJ, grpE, groES, groEL, tig, fkpA, surA, skp, PpiD and DegP.

In certain embodiments, the genetically engineered probiotic intestinal bacterium further comprises a Chaperon expression cassette comprising at least one nucleotide sequence that encodes at least one Chaperone protein selected from the group consisting of: dsbA, dsbC, dnaK, dnaJ, grpE, groES, groEL, tig, fkpA, surA, skp, PpiD and DegP.

Chaperone proteins are involved in many important biological processes such as protein folding and aggregation of oligomeric protein complexes, maintaining protein precursors in an unfolded state to facilitate protein transmembrane transport, and enabling denatured proteins to be disaggregated and repaired. It is mainly to assist other peptides to maintain the normal conformation to form the correct oligomeric structure, thereby exerting normal physiological functions. Various Chaperon proteins are well-known in the art. In some embodiments, the Chaperone protein is selected from the group consisting of Ssa1p, Ssa2p, Ssa3p and Ssa4p from the cytosolic SSA subfamily of 70 kDa heat shock proteins (Hsp70), BiP, Kar2, Lhs1, Sil1, Sec63, Protein disulfide isomerase Pdilp.

### 5. Genome Integration Site

In certain embodiments, the exogenous expression cassette is integrated in the genome of the genetically engineered probiotic intestinal bacterium. In some embodiments, the exogenous expression cassette is integrated into the genome of the genetically engineered probiotic intestinal bacterium by CRISPR-Cas genome editing system. Any suitable host cells provided herein can be engineered such that the exogenous expression cassette is integrated into the genome. Various genome integration sites can be selected so long as the heterogeneous gene will be expressed at certain amount and will have no major negative impact on the chassis probiotic intestinal bacterium's biochemical and physiological activity.

In some embodiments, the exogenous expression cassette is integrated in the EcN genome at an integration site selected from those listed in Table 2. In some embodiments, the suitable integration site integrated with the exogenous expression cassette in the EcN genome is kefB. Without wishing to be bound by any theory, but it is believed that the genome sites of EcN listed in Table 2 are advantageous in at least one of the following characteristics for insertion of the expression cassette for AcoD: (1) the bacterial gene(s) impacted by the site's engineering are not essential for EcN's growth and do not change the host bacteria biochemical and physiological activity, (2) the site can be easily edited, and (3) the AcoD gene cassette in the site can be transcribed. The sgRNA sequences used to edit corresponding genome sites in EcN are shown in Table 2 below.

TABLE 2
sgRNA sequence used to edit corresponding genome
site in EcN.

| | | SEQ |
|---|---|---|
| | | ID |
| Integration site | sgRNA | NO. |
| agaI/rsmI (~3645 kbp) | ggcgagttaacgacgacaca | 70 |
| araBC (~69 kbp) | cgttgaactgggtgtggaat | 71 |
| cadA (~4822 kbp) | ctgaaaccgctgcggcgatg | 72 |
| cadA (~4822 kbp) | gttcgcagtggaagtaccgt | 73 |
| dapA (~2842 kbp) | ctgtgcaaacaagtgtctca | 74 |
| kefB (~3840 kbp) | gccggaagacactatgaagc | 75 |
| lacZ (~450 kbp) | tcgcacagcgtgtaccacag | 76 |
| maeB (~2819 kbp) | gaaggggaagaggcgcgcgt | 77 |
| malE/K: (~4687 kbp) | cggtttagttcacagaagcc | 78 |
| malP/T (~3908 kbp) | ttgcgtattttcaaaaagcg | 79 |
| rhtB/C: (~4409 kbp) | tcatcagagtaagtcggata | 80 |
| yicS/nepI (~4241 kbp) | ctgaccaacgcttctttacc | 81 |
| adhE (~1467 kbp) | ccgaagtccctgtgtgcttt | 82 |
| galK (~816 kbp) | ccctgccactcacaccattc | 83 |
| glk (~2763 kbp) | ccttctcctggcaagcttac | 84 |
| ldhA (~1586 kbp) | cgacaagaagtacctgcaac | 85 |
| lldD (~4148 kbp) | gttatcgtgatgcgcattct | 86 |
| maeA (~1675 kbp) | taacacccagcccgatgccc | 87 |
| nth (~1800 kbp) | atattactggaacaacataa | 88 |
| pflB (~975 kbp) | ccgtgacgttatccgcacca | 89 |
| rnC (~2939 kbp) | tatcgccttcatccacacga | 90 |
| tkrA(ghrB) (~4090 kbp) | ccgggctggatgtcttcgaa | 91 |
| yieN (ravA) (~2327 kbp) | gagggtgagccataatgaag | 92 |
| yjcS (~4772 kbp) | ggatatgtggggtaacgacg | 93 |
| LPP(~1846 kbp) | acgttcaggctgctaaagat | 94 |

### 6. Auxotroph

In certain embodiments, the genetically engineered probiotic intestinal bacterium further comprises at least one inactivation or deletion in an auxotroph-related gene.

In order to generate an environment-friendly bacteria, some essential genes that necessary for bacterial cell survival can be deleted or inactivated by mutagenesis, making the engineered bacteria become auxotroph. The term “auxotroph” as used herein refers to a host cell (e.g. a strain of microorganism) requiring for growth an external source of a specific metabolite that cannot be synthesized because of an acquired genetic defect. The term “auxotroph-related gene” as used herein refers to a gene required for the host cell (e.g. microorganism such as bacteria) to survive. The auxotroph-related gene can be necessary for the microorganism to produce for a nutrient essential for survival or growth, or can be required for detection of a signal in an environment that modulates activity of the transcription factor, wherein absence of the signal would lead to the cell death.

In some embodiments, an auxotrophic modification is intended to cause the microorganism to die in the absence of an exogenously added nutrient essential for survival or growth because they lack the gene(s) necessary to produce that essential nutrient. In some embodiments, any of the genetically engineered bacteria described herein also comprise a deletion or mutation in a gene required for cell survival and/or growth.

Various auxotroph-related genes in bacteria are well-known in the art. Exemplary auxotroph-related genes include, but not limited to, thyA, cysE, glnA, ilvD, leuB, lysA, serA, metA, glyA, hisB, ilvA, pheA, proA, thrC, trpC, tyrA, uraA, dapF, flhD, metB, metC, proAB, yhbV, yagG, hemB, secD, secF, ribD, ribE, thiL, dxs, ispA, dnaX, adk, hemH, IpxH, cysS, fold, rplT, infC, thrS, nadE, gapA, yeaZ, aspS, argS, pgsA, yeflA, metG, folE, yejM, gyrA, nrdA, nrdB, folC, accD, fabB, gltX, ligA, zipA, dapE, dapA, der, hisS, ispG, suhB, tadA, acpS, era, rnc, fisB, eno, pyrG, chpR, Igt, ft>aA, pgk, yqgD, metK, yqgF, plsC, ygiT, pare, ribB, cca, ygjD, tdcF, yraL, yihA, ftsN, murl, murB, birA, secE, nusG, rplJ, rplL, rpoB, rpoC, ubiA, plsB, lexA, dnaB, ssb, alsK, groS, psd, orn, yjeE, rpsR, chpS, ppa, valS, yjgP, yjgQ, dnaC, ribF, IspA, ispH, dapB, folA, imp, yabQ, flsL, flsl, murE, murF, mraY, murD, ftsW, murG, murC, ftsQ, ftsA, ftsZ, IpxC, secM, secA, can, folK, hemL, yadR, dapD, map, rpsB, in/B, nusA, ftsH, obgE, rpmA, rplU, ispB, murA, yrbB, yrbK, yhbN, rpsl, rplM, degS, mreD, mreC, mreB, accB, accC, yrdC, def, fint, rplQ, rpoA, rpsD, rpsK, rpsM, entD, mrdB, mrdA, nadD, hlepB, rpoE, pssA, yfiO, rplS, trmD, rpsP, ffh, grpE, yfjB, csrA, ispF, ispD, rplW, rplD, rplC, rpsJ, fusA, rpsG, rpsL, trpS, yr/F, asd, rpoH, ftsX, ftsE, ftsY, frr, dxr, ispU, rfaK, kdtA, coaD, rpmB, djp, dut, gmk, spot, gyrB, dnaN, dnaA, rpmH, rnpA, yidC, tnaB, glmS, glmU, wzyE, hemD, hemC, yigP, ubiB, ubiD, hemG, secY, rplO, rpmD, rpsE, rplR, rplF, rpsH, rpsN, rplE, rplX, rplN, rpsQ, rpmC, rplP, rpsC, rplV, rpsS, rplB, cdsA, yaeL, yaeT, lpxD, fabZ, IpxA, IpxB, dnaE, accA, tilS, proS, yafF, tsf, pyrH, olA, rlpB, leuS, Int, glnS, fldA, cydA, in/A, cydC, ftsK, lolA, serS, rpsA, msbA, IpxK, kdsB, mukF, mukE, mukB, asnS, fabA, mviN, rne, yceQ, fabD, fabG, acpP, tmk, holB, lolC, lolD, lolE, purB, ymflC, minE, mind, pth, rsA, ispE, lolB, hemA, prfA, prmC, kdsA, topA, ribA, fabi, racR, dicA, yd B, tyrS, ribC, ydiL, pheT, pheS, yhhQ, bcsB, glyQ, yibJ, and gpsA.

In one modification, the essential gene thyA is deleted or replaced by another gene making the genetically engineered bacteria dependent on exogenous thymine to grow or survive.

Adding thymine to growth media or the human gut naturally having high thymine level can support the growth and survival of thyA auxotroph bacteria. This kind of modification is to ensure that the genetically engineered bacteria cannot grow and survive outside of the gut or in the environment that in lack of the auxotrophic gene product.

In some embodiments, the probiotic intestinal bacterium is an auxotroph for one or more substances selected from the group consisting of thymidine, uracil, leucine, histidine, tryptophan, lysine, methionine, adenine, and non-naturally occurring amino acid. In some embodiments, the non-naturally occurring amino acid is selected from the group consisting of 1-4,4′-biphenylalanine, p-acetyl-1-phenylalanine, p-iodo-1-pheylalanine, and p-azido-1-phenylalanine.

In some embodiments, the probiotic intestinal bacterium comprises an allosterically regulated transcription factor which is capable of detecting a signal in an environment that modulates activity of the transcription factor, wherein absence of the signal would lead to the cell death. Such “signaling molecule—transcription factor” pairs may include any one or more selected from the group consisting of tryptophan-TrpR, IPTG-LacI, benzoate derivatives-XylS, ATc-TetR, galactose-GalR, estradiol-estrogen receptor hybrid protein, cellobiose-CelR, and homoserine lactone-luxR.

### Recombinant Expression Cassette

In another aspect, the present disclosure also provides a recombinant expression cassette comprising a nucleotide sequence that encodes AcoD, and one or more regulatory elements, wherein the nucleotide sequence has been optimized for expression in EcN, and optionally, the codon-optimized nucleotide sequence comprises a sequence of SEQ ID NO: 111 or a homologous sequence thereof having at least 80% sequence identity.

The term “expression cassette” as used herein refers to a DNA sequence capable of directing expression of a particular nucleotide sequence in an appropriate probiotic intestinal bacterium, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a non-translated RNA, in the sense of antisense direction. The expression cassette comprising the nucleotide sequence of interest may be chimeric, meaning that at least one of its components is heterologous with respect to at least one of its other components.

The expression cassette is suitable for expressing the AcoD polypeptide in the probiotic intestinal bacterium provided herein. The expression cassette may be introduced as part of a nucleic acid vector (e.g. an expression vector such as those described above). Suitable vectors for probiotic intestinal bacteria can include plasmids. A vector may include sequences flanking the expression cassette that include sequences homologous to eukaryotic genomic sequences, such as mammalian genomic sequences, prokaryotic genomic sequences, such as bacterial genomic sequences, or viral genomic sequences. This will allow the introduction of the expression cassette into the genome of eukaryotic cells, prokaryotic genomic sequences or viruses by homologous recombination.

The term “recombinant” as used herein refers to a polynucleotide synthesized or otherwise manipulated in vitro (e.g., “recombinant polynucleotide” or “recombinant expression cassette”), to methods of using recombinant polynucleotides or recombinant expression cassette to produce products in cells or other biological systems, or to a polypeptide (“recombinant protein”) encoded by a recombinant polynucleotide. Recombinant polynucleotides encompass nucleic acid molecules from different sources ligated into an expression cassette or vector for expression of, e.g., a fusion protein; or those produced by inducible or constitutive expression of a polypeptide (e.g., an expression cassette or vector of the invention operably linked to a heterologous polynucleotide, such as an AcoD coding sequence). Recombinant expression cassette encompasses a recombinant polynucleotide operably linked to one or more regulatory elements.

In some embodiments, the recombinant expression cassette further comprises one or more regulatory elements selected from the group consisting of: a promoter, a ribosome binding site (RBS), a terminator, and any combination thereof. In some embodiments, the promoter is a constitutive promoter, or an inducible promoter (e.g., an anaerobic inducible promoter). The promoter can be an endogenous promoter, or an exogenous promoter. In some embodiments, the promoter comprises a nucleotide sequence selected from the group consisting of SEQ ID NOs: 10-49 and homologous sequences thereof having at least 80% sequence identity. In some embodiments, the promoter comprises SEQ ID NO: 10.

In some embodiments, the RBS comprises a nucleotide sequence selected from the group consisting of SEQ ID NOs: 65-67 and homologous sequences thereof having at least 80% sequence identity. In some embodiments, the terminator is T7 terminator.

### III. Compositions

In another aspect, the present disclosure also provides a composition comprising the genetically engineered probiotic intestinal bacterium expressing the AcoD or functional equivalents thereof, and a physiologically acceptable carrier. The carrier may be any compatible, physiologically-acceptable, non-toxic substances suitable to deliver the genetically engineered probiotic intestinal bacterium provided herein to the gastrointestinal (GI) tract of a mammal (e.g. human) in a mammal. In certain embodiments, the composition further comprises one or more physiologically acceptable carrier selected from lactic acid fermented foods, fermented dairy products, resistant starch, dietary fibers, carbohydrates, fat, oil, flavoring agent, seasoning agent, proteins and glycosylated proteins, water, capsule filler, and a gummy material.

In certain embodiments, the composition is edible. In certain embodiments, the composition is a food supplement. In certain embodiments, the composition is formulated as functional food such as drinks, fermented yoghurts, etc.

In certain embodiments, the composition is a pharmaceutical composition. In certain embodiments, the compositions can also be formulated as medicaments, in capsules, pills, liquid solution, for example as encapsulated lyophylized bacteria etc.

In certain embodiments, the composition may be in liquid form, for example, such as elixirs, syrups, and suspensions; or in solid form, for example, such as capsules, tablets, and powders.

In certain embodiments, the composition comprises a powder of lyophilized bacteria cells. Cryoprotectant such as lactose, trehalose or glycogen may be employed for lyophilized bacteria cells.

In certain embodiments, the genetically-engineered microorganism is a live cell. In certain embodiments, the composition is a finished food product, a powder, a granule, a tablet, a capsule, or a liquid. In certain embodiments, the composition comprises about 0.01 to about 99.9%, about 10.01 to about 89.9%, about 20.01 to about 79.9%, about 30.01 to about 69.9%, about 40.01 to about 69.9%, or about 5.01 to about 59.9% by weight genetically-engineered microbe.

The compositions disclosed herein may be formulated to be effective in a given subject in a single administration or over multiple administrations. For example, a single administration is substantially effective to reduce a monitored symptom of a targeted disease or condition, e.g., hangover, alcoholic liver disease, or to effectively prevent symptoms of a targeted disease or condition, e.g., hangover, alcoholic liver disease, or to effectively prevent progression of a targeted disease or condition, in a mammalian subject to whom the composition is administered.

Generally, the dosage of recombinant bacteria will vary depending upon such factors as the subject's age, weight, height, sex, general medical condition and previous medical history. In some embodiments, the composition is formulated such that a single oral dose contains at least about 1×10⁴ CFU of the bacterial entities and/or fungal entities, and a single oral dose will typically contain about 1×10⁴, 1×10⁵, 1×10⁶, 1×10⁷, 1×10⁸, 1×10⁹, 1×10¹⁰, 1×10¹¹, 1×10¹², or 1×10¹³ CFUs of the bacterial entities and/or fungal entities. In some embodiments, the composition is formulated such that a single oral dose contains no more than about 1×10¹³ CFU of the bacterial entities and/or fungal entities. If known, for example the concentration of cells of a given strain, or the aggregate of all strains, is about e.g., 1×10⁴, 1×10⁵, 1×10⁶, 1×10⁷, 1×10⁸, 1×10⁹, 1×10¹⁰, 1×10¹¹, 1×10¹², or 1×10¹³ viable bacterial entities (e.g., CFUs) per gram of composition (optionally dry composition) or per administered dose. In certain embodiments, the concentration of cells of a given strain, or the aggregate of all strains, is no more than 1×10¹³ viable bacterial entities (e.g., CFUs) per gram of composition (optionally dry composition) or per administered dose.

In some formulations, the composition contains at least or at least about 0.5%, 1%, 2%, 5%, 10%, 20%, 30%, 40%, 50%, 60%, 70%, 80%, 90% or greater than 90% probiotic intestinal bacteria of the present disclosure on a mass basis. In some formulations, the administered dose does not exceed 200, 300, 400, 500, 600, 700, 800, 900 milligrams or 1, 1.1, 1.2, 1.3, 1.4, 1.5, 1.6, 1.7, 1.8, or 1.9 grams of probiotic intestinal bacteria of the present disclosure in mass.

### IV. Method of Use

i. Alcohol Hangover

The present disclosure provides therapeutic uses of the genetically engineered probiotic intestinal bacterium, and/or the composition comprising the genetically engineered probiotic intestinal bacterium provided herein. Acetaldehyde is a highly soluble molecule and can passively diffuse across the cellular membrane of the genetically engineered bacteria provided herein to serve as a substrate for the AcoD expressed inside the engineered bacterium to be oxidized into acetate. The internal localization of the enzyme is more advantageous than the enzymes secreted from the bacterium, as the secreted enzyme would have to face harsh and variable environment in the lumen of the gut, e.g., low pH, hostile bacteria and eukaryotic cells that are looking to degrade free floating proteins for defense or nutritional purposes, high competition for enzymatic co-factors such as NAD, and extracellular proteases, whereas the enzymes expressed and functions inside the bacterial cell can be protected from the unpleasant environment and thus significantly improves the activity and efficacy in acetaldehyde removal.

In one aspect, the present disclosure provides methods for preventing and/or treating an alcohol hangover in a subject in need thereof, comprising administering to the gut of the subject an effective amount of the genetically engineered probiotic intestinal bacterium or the composition provided herein. The term “hangover” as used herein refers to a collection of unpleasant signs and symptoms including, without limitation, fatigue and weakness, excessive thirst and dry mouth, headaches and muscle aches, nausea, vomiting or stomach pain, poor or decreased sleep, increased sensitivity to light and sound, dizziness or a sense of the room spinning, shakiness, decreased ability to concentrate, mood disturbances (such as depression, anxiety and irritability), and rapid heartbeat. The hangover can be a result of overdrinking and/or fast drinking that leads to accumulation of acetaldehyde in blood. “Overdrinking” as used herein means drinking an amount of alcohol beyond the alcohol tolerance of a person. The alcohol tolerance varies among population depending on the genetic conditions of the population. For example, single nucleotide polymorphisms in acetaldehyde dehydrogenase genes (e.g., ALDH2 variant alleles) common in East Asian populations reduces alcohol tolerance or even leads to alcohol intolerance, which causes accumulation of acetaldehyde in the body.

The term “acetaldehyde” as used herein refers to a toxic intermediate in an alcohol metabolic pathway produced by oxidizing alcohol via alcohol dehydrogenase enzymes. Acetaldehyde can be subsequently oxidized in the liver to acetate via acetaldehyde dehydrogenase enzymes. Accumulation of acetaldehyde is cause of many of the effects of an alcohol hangover. Without wishing to be bound by any theory, it is believed that promoting acetaldehyde metabolism by introducing exogenous catalytically active acetaldehyde dehydrogenases would prevent and/or reduce symptoms of alcohol hangover. Therefore, administering the genetically engineered probiotic intestinal bacterium or the composition of the present disclosure into the gut of a subject that produces exogenous acetaldehyde dehydrogenases with catalytic activities in oxidizing acetaldehyde into acetate, can effectively reduce and/or prevent symptoms of hangover.

Acetaldehyde is also known as a carcinogen, whose toxic effects are a well-studied and documented. For example, it damages the epithelial barrier and increases the permeability of the epithelial layer in the intestinal tract (Chaudhry K K et al., Alcoholism, clinical and experimental research. 2015; 39:1465-75). Increased accumulation of acetaldehyde in hepatocytes can also result in liver fibrosis, which has shown to be associated with inactive ALDH2 (Purohit V et al., Hepatology (Baltimore, Md). 2006; 43:872-8). Studies have shown that overexpression of ALDH2 could attenuate chronic alcohol-induced liver damage and apoptosis (Guo et al., Clinical and Experimental Pharmacology and Physiology. 2009; 36:463-8.). Accordingly, the present disclosure also provides methods for reducing levels of acetaldehyde in a subject in need thereof, comprising administering to the gut of the subject an effective amount of the genetically engineered probiotic intestinal bacterium or the composition provided herein.

In another aspect, the present disclosure provides methods for preventing and/or treating Asian flush in a subject in need thereof, comprising administering to the gut of the subject an effective amount of the genetically engineered probiotic intestinal bacterium or the composition provided herein. The term “Asian flush” as used herein refers to a face flushing response to alcohol consumption, which is often observed in Asian population. Asian flush can be a defensive mechanism that may deter alcohol consumption. However, in social events where people are encouraged or challenged to drink more alcohol, individuals with Asian flush may not be able to escape or decline this drinking binge. Therefore, a preventive and/or therapeutic method for Asian flush is needed in scenarios where social drinking is inevitable.

Asian flush is generally associated with deficient in one or more alcohol dehydrogenases, e.g., aldehyde dehydrogenases. In certain embodiments, the subject is deficient in one or more acetaldehyde dehydrogenases. In certain embodiments, the subject is deficient in acetaldehyde dehydrogenases 2 (ALDH2). In certain embodiments, the subject is a carrier of ALDH2 variant alleles. As used herein, the term “ALDH2 variant allele” can refer to an ALDH2 allele that comprises a functional single nucleotide polymorphism (SNP), e.g., in exon 12, which results in an E487K substitution, ie., ALDH2*487 Lys, also named ALDH2*2. The ALDH2*2 encodes a functionally deficient version of the mitochondrial ALDH2 enzyme, which leads to catalytic inactivation of ALDH2 (Agarwal, Pathol Biol (Paris). 2001 Novemmber; 49(9):703-9.; Ramchandani et al., Pathol Biol (Paris). 2001 November; 49(9):676-82.; Vasiliou et al., Pharmacology. 2000 September; 61(3):192-8; Yoshida, Pharmacogenetics. 1992 August; 2(4):139-47). The term “ALDH2 variant allele” can also comprise ALDH2*1. The enzyme encoded by the ALDH2*1/*2 is partially inactive, and the enzyme encoded by the ALDH2*2/*2 is completely inactive. In certain embodiments, the subject is a carrier of ALDH2*1/*2. In certain embodiments, the subject is a carrier of ALDH2*2/*2.

The ALDH2 deficiency can be detected at an enzymatic activity level and/or a genetic level. The ALDH2 deficiency at an enzymatic activity level can be measured by any suitable functional assay known in the art. The ALDH2 deficiency at a genetic level can be measured by any methods known in the art, for example, without limitation, an amplification assay, a hybridization assay, or a sequencing assay.

iii. Alcoholic Liver Disease

Alcohol metabolism mainly takes place in the liver. In addition to the detoxification effect on acetaldehyde as mentioned above, acetaldehyde dehydrogenases have also been shown to be involved in pathogenesis of liver disease. While ALDH2*2 may protect a subject from getting alcoholic liver disease (ALD) as its associated Asia flush would highly likely prevent the subject from consuming alcohol, such protection against ALD by the ALDH2*2 allele can wane over time by more alcohol consumption, which increases alcohol tolerability (Higuchi S et al., The Lancet. 1994; 343:741-2.). Therefore, a subject carrying ALDH2*2 alleles can still develop ALDs as long as consumption of alcohol is not avoided.

In another aspect, the present disclosure provides methods for preventing and/or treating alcoholic liver disease (ALD) in a subject in need thereof, comprising administering to the gut of the subject an effective amount of the genetically engineered probiotic intestinal bacterium or the composition provided herein. ALD is a complex process that includes a wide spectrum of hepatic lesions, from steatosis to cirrhosis. Cell injury, inflammation, oxidative stress, regeneration and bacterial translocation are key drivers of alcohol-induced liver injury. The prevalence rates of ALD were reported to be 4.5%, 6.2%, 6% and 1.56-2.34% in China, the US, Europe, and Japan, respectively (Xiao Jet al., Journal of hepatology. 019; 71:212-21; Fan J-G eg al., Journal of Gastroenterology and Hepatology. 2013; 28:11-7; Rehm Jet al., The Lancet. 2009; 373:2223-33; and Szabo G et al., Hepatology (Baltimore, Md). 2019; 69:2271-83.). In certain embodiments, the alcoholic liver disease is alcoholic fatty liver, alcoholic hepatitis, alcoholic fibrosis or alcoholic liver cirrhosis. Alcoholic fatty liver is an initial stage of ALD that can progress to alcoholic hepatitis with inflammation. The alcoholic hepatitis can progress to alcoholic liver fibrosis, which can further progress to alcoholic liver cirrhosis and then alcoholic liver cancer. These disorders not only develop sequentially from fatty liver to alcoholic hepatitis to fibrosis to cirrhosis, but can also occur together.

In another aspect, the present disclosure provides methods for preventing and/or slowing down progression of alcoholic fatty liver disease into alcoholic liver fibrosis, alcoholic liver cirrhosis or alcoholic liver cancer in a subject in need thereof, comprising administering to the gut of the subject an effective amount of the genetically engineered probiotic intestinal bacterium or the composition provided herein.

In another aspect, the present disclosure provides methods for preventing and/or slowing down progression of alcoholic hepatitis into alcoholic liver fibrosis, alcoholic liver cirrhosis or alcoholic liver cancer in a subject in need thereof, comprising administering to the gut of the subject an effective amount of the genetically engineered probiotic intestinal bacterium or the composition provided herein.

In another aspect, the present disclosure also provides a method for preventing and/or treating non-alcoholic fatty liver (NAFLD) or non-alcoholic steatohepatitis (NASH) in a subject in need thereof, comprising administering to the gut of the subject an effective amount of the genetically engineered probiotic intestinal bacterium or the composition provided herein. In certain embodiments, the NAFLD is steatosis, non-alcoholic steatohepatitis (NASH), cirrhosis or liver cancer.

As used herein, “non-alcoholic fatty liver disease (NAFLD)” is an all-encompassing term used to describe the fatty liver environment in the absence of excessive alcohol consumption. It is estimated that 25% of the world's general population meet the criteria for a diagnosis of NAFLD, which is more common in men and increases with age.

The initial stage of NAFLD can be detected based on the characteristics, such as the accumulation of ectopic fat in hepatocytes (steatosis). Steatosis is generally a benign, asymptomatic condition; however, with concurrent obesity/metabolic disturbances, steatosis can progress to non-alcoholic steatohepatitis (NASH) that has increased risk for liver fibrosis and in severe cases hepatocellular carcinoma (HCC), and liver failure.

NASH can be detected histologically by characteristics, such as hepatocellular ballooning and inflammation. Unlike benign steatosis, NASH represents a significant health threat that progresses to fibrosis/cirrhosis in 10-28% of patients. Further progression from NASH to fibrosis/cirrhosis is highly predictive of mortality in these patients.

Studies have found that in spite of alcohol-deficient diet, a patient having NAFLD progressed to NASH is still associated with elevated level of alcohol in the patient's systemic circulation and breath (i.e., endogenous alcohol or gut-bacteria-derived ethanol) and increased gene transcription of alcohol dehydrogenase genes. Such alcohol may be produced from carbohydrate fermentation by alcohol-producing microbiota (e.g., Escherichia, Ruminococcus, Klebsiella pneumonia) inside the patient and the endogenous alcohol may be involved in NAFLD progression via direct toxic effects on hepatic cells via impairments in gut barrier function that leads to increased portal endotoxaemia, and via the upregulation of nuclear factor-κB (NF-κB) signaling pathways in peripheral cells (Zhu et al., Hepatology. 2013 February; 57(2):601-9;Canfora et al., Nat Rev Endocrinol. 2019 May; 15(5):261-273; and Yuan et al., 2019, Cell Metabolism 30, 675-688).

Studies have also shown significant oxidative stress and reduced ALDH activity as suggested by significant accumulation of 4-HNE protein adduct in NASH. 4-HNE is a covalent modification of an ALDH2 active site peptide and is reported to be a potent irreversible inhibitor of ALDH2, which indicates that inactivation of ALDH2 by 4-HNE may be a cause of NASH (Li et al., Toxicological sciences: an official journal of the Society of Toxicology. 2018; 164:428-38; and Doorn et al., Chemical research in toxicology. 2006; 19:102-10). Accordingly, the present disclosure also provides a method for preventing and/or slowing progression of NAFLD into NASH in a subject in need thereof, comprising administering to the gut of the subject an effective amount of the genetically engineered probiotic intestinal bacterium or the composition provided herein. In another aspect, the present disclosure also provides a method for preventing and/or slowing progression of NASH into liver fibrosis in a subject in need thereof, comprising administering to the gut of the subject an effective amount of the genetically engineered probiotic intestinal bacterium or the composition provided herein. A NAFLD/NASH patient can be benefit from the genetically engineered probiotic intestinal bacterium or the composition of the present disclosure, which has been shown to effectively degrade acetaldehyde in vitro as well as in vivo, compensating the inactivated ALDH2 in the NASH patient so as to prevent accumulation of toxic acetaldehyde in the body.

In certain embodiments, the subject has an elevated level of blood ethanol or serum ethanol relative to a reference level. As used herein, the term “reference level” with respect to blood ethanol or serum ethanol refers to a benchmark level which allows for comparison. A reference level may be chosen by the persons skilled in the art according to the desired purpose. Means for determining suitable reference levels are known to the persons skilled in the art, e. g. a reference level can be determined from experience, existing knowledge or data collected from clinical studies. For example, the reference level of blood alcohol can be the level of blood alcohol in a normal healthy person with the same gender and comparable body weight, and optionally having other factors that are also comparable, such as, the physical condition, medication history, diet, sleep, etc. For example, as reported by Zhu et al (Hepatology, 57(2):2013, page 601-609), the serum ethanol level is about 25 μM in healthy subjects, but is about 35 μM in NASH patients. In certain embodiments, the subject has a serum ethanol level at least 10%, 15%, 20%, 25%, or 30% higher than that of a reference level.

In certain embodiments, the subject has increased abundance of alcohol-producing gut microbiota relative to a reference level. In certain embodiments, the increased abundance of alcohol-producing gut microbiota refers to an elevated amount of alcohol-producing microbiota relative to a reference level or amount of such microbiota in a healthy person. The increased abundance of alcohol-producing gut microbiota may also refer to the enhanced capability of producing alcohol for the alcohol-producing gut microbiota in a patient as compared to that in a healthy person. For example, the alcohol-producing gut microbiota can be a common gut microbiota that produce more alcohol (either due to an increased amount of such microbiota or due to its enhanced capability of alcohol producing) in an abnormal condition, e.g., in a NASH patient, than in a healthy person. Exemplary alcohol-producing gut microbiota include without limitation, Klebsiella pneumonia, Escherichia, Bacteroides, Bifidobacterium, Clostridium, and yeast (Yuan et al., 2019, Cell Metabolism 30, 675-688; Frantz J C et al., J Bacteriol 1979;137:1263-1270; Zhu et al., Hepatology. 2013 February; 57(2):601-9; Amaretti A et al., Appl Environ Microbiol 2007; 73:3637-3644; and Weimer P J et al., Appl Environ Microbiol 1977;33:289-297.). The abundance of alcohol-producing gut microbiota in a subject can be measured by, for example, assaying the alcohol concentrations produced by the fecal samples isolated from the subject after being fermented anaerobically or aerobiclly in suitable medium containing carbohydrates, such as fructose, or glucose (Yuan et al., 2019, Cell Metabolism 30, 675-688). The abundance of alcohol-producing gut microbiota in a subject can also be measured by isolating genomic DNA from fecal samples of a subject, sequencing (e.g., 16S ribosomal RNA pyrosequencing) the genomic DNA, and followed by identification, classification and abundance analysis of microbiota composition (Zhu et al., Hepatology. 2013 February; 57(2):601-9). To determine if the abundance is increased, the alcohol concentrations produced by the fecal sample from a subject measured according to the method mentioned above or the microbiota abundance result from a subject measured and analyzed according to the method mentioned above can be compared with that in a normal subject.

In certain embodiments, the composition is administered before, during, or after consumption of alcohol. In certain embodiments, the composition is administered to the subject up to 24 hours before commencement of consumption of alcohol.

Hangover prevention can be achieved by administering the composition provided herein to a subject before (e.g., up to any of 24, 20, 18, 16, 14, 12, 10, 8, 6, 4, 2, 1 or 0.5 hours before) alcohol consumption. Hangover treatment and/or mitigation can be achieved by administering the composition provided herein during and/or after consumption of alcohol or any time when a subject develops symptoms of hangover. For example, the composition can be administered up to any of 24, 20, 18, 16, 14, 12, 10, 8, 6, 4, 2, 1 or 0.5 hours after alcohol consumption.

Prevention and/or treatment of alcoholic liver disease or non-alcoholic fatty liver may be achieved by administering the composition provided herein to a subject at regular intervals (e.g., once daily, twice a day, three times a day, etc. for a certain period).

ALDH2 variant alleles have also been found to be associated with several other diseases or pathophysiological conditions, including without limitation, gastric cancers, Alzheimer's, osteoporosis, myocardial infarction, hypertension, esophageal and head & neck cancers. Accordingly, the present disclosure also provides methods for preventing, treating and/or slowing down progression of any of the diseases or pathophysiological conditions mentioned above, comprising administering to the gut of the subject an effective amount of the genetically engineered probiotic intestinal bacterium or the composition provided herein.

In another aspect, the present disclosure also provides use of an effective amount of the genetically engineered probiotic intestinal bacterium or the composition provided herein in the manufacture of a medicament for preventing and/or treating an alcohol hangover in a subject in need thereof.

In another aspect, the present disclosure also provides use of an effective amount of the genetically engineered probiotic intestinal bacterium or the composition provided herein in the manufacture of a medicament for reducing levels of acetaldehyde in a subject in need thereof.

In another aspect, the present disclosure also provides use of an effective amount of the genetically engineered probiotic intestinal bacterium or the composition provided herein in the manufacture of a medicament for preventing and/or treating Asian flush in a subject in need thereof.

In another aspect, the present disclosure also provides use of an effective amount of the genetically engineered probiotic intestinal bacterium or the composition provided herein in the manufacture of a medicament for preventing and/or treating alcoholic liver disease in a subject in need thereof.

In another aspect, the present disclosure also provides use of an effective amount of the genetically engineered probiotic intestinal bacterium or the composition provided herein in the manufacture of a medicament for preventing and/or slowing down progression of alcoholic fatty liver disease into alcoholic liver fibrosis, alcoholic liver cirrhosis or alcoholic liver cancer in a subject in need thereof.

In another aspect, the present disclosure also provides use of an effective amount of the genetically engineered probiotic intestinal bacterium or the composition provided herein in the manufacture of a medicament for preventing and/or slowing down progression of alcoholic hepatitis into alcoholic liver fibrosis, alcoholic liver cirrhosis or alcoholic liver cancer in a subject in need thereof.

In another aspect, the present disclosure also provides use of an effective amount of the genetically engineered probiotic intestinal bacterium or the composition provided herein in the manufacture of a medicament for preventing and/or treating non-alcoholic fatty liver (NAFLD) or non-alcoholic steatohepatitis (NASH) in a subject in need thereof.

In another aspect, the present disclosure also provides use of an effective amount of the genetically engineered probiotic intestinal bacterium or the composition provided herein in the manufacture of a medicament for preventing and/or slowing progression of NAFLD into NASH in a subject in need thereof.

In another aspect, the present disclosure also provides use of an effective amount of the genetically engineered probiotic intestinal bacterium or the composition provided herein in the manufacture of a medicament for preventing and/or slowing progression of NASH into liver fibrosis in a subject in need thereof.

### EXAMPLES

### Example 1. Preparation of Guide RNA (gRNA) Plasmid ZL-003_kefB

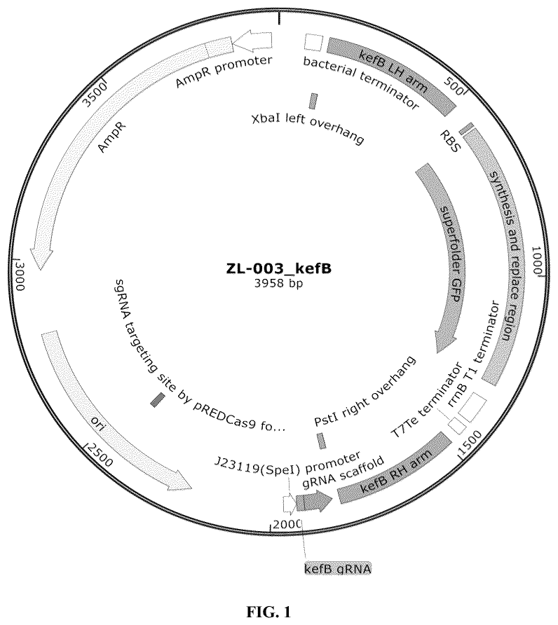

The schematic map of the gRNA plasmid ZL-003_kefB is shown in FIG. 1, which was constructed using conventional methods known in the art. Briefly, a 20-bp sequence together with NGG PAM sequence (N20NGG) was searched on both strands of the target integration sequence and blasted against the EnN genome. The unique 20-bp sequences were selected as sgRNA of the target integration sites. A 300-500 bp sequence upstream and downstream of the sgRNA were selected as the left homologous arm (LHA) and right homologous arm (RHA). The sgRNA sequence was added to the 5′ end of the reverse primer of the gRNA scaffold on the ZL-003 plasmid, then the gRNA scaffold together with the designed sgRNA sequence was amplified from ZL-003, both the PCR product and the ZL-003 plasmids were digested, and the digested ZL-003 plasmid was dephosphorylated, and then ligated with the digested PCR fragments to generate the gRNA plasmid ZL-003_kefB.

### Example 2. Design of Donor Gene Cassettes (kefB_J23119_AcoD, kefB_J23101_AcoD and kefB_J23108_AcoD)

The target gene to be integrated to the genome of EcN, i.e., acetaldehyde dehydrogenase gene AcoD, was derived from Cupriavidus necator. The amino acid sequence of AcoD is shown in SEQ ID NO: 1. The target gene was synthesized on a cloning plasmid (e.g. pUC57) by GeneScript (pUC57_AcoD, SEQ ID NO: 2). The target gene was amplified from the plasmid pUC57_AcoD, LHA and RHA of the selected integration sites were amplified from the genome of EcN. The primers used for PCR of these fragments had 15-20 bp homologous sequence with each other, so that they can be ligated by overlap PCR with the target gene flanked by LHA and RHA. The linear PCR product was used as donor gene cassette.

In particular, primers used for the PCR of EcN_kefB_J23119_AcoD cassette are listed in Table 3 below respectively. Primers 1 and 2 were used to amplify the AcoD fragment from the plasmid pUC57-AcoD (synthesized by Shanghai Sunny Biotechnology Co., Ltd.) using high-fidelity thermostable DNA polymerases. Primers 3 and 4 were used to amplify the terminator-kefB RH arm fragment from the plasmid ZL-003_kefB_J23119-GFP synthesized in the lab. Primer 5 and 6 were used to amplify the KefB LH arm-J23119-RBS fragment from the plasmid ZL-003_kefB_J23119-GFP synthesized in the lab. Finally Primers 5 and 4 were used to amplify the kefB_J23119-AcoD (SEQ ID NO: 7) fragment using the AcoD fragment, the terminator-kefB RH arm fragment, and the KefB LH arm-J23119-RBS fragment as templates. The kefB_J23101_AcoD fragment and the kefB_J23108_AcoD fragment were synthesized in the same manner except that different promoters were used.

TABLE 3
kefB_J23119-AcoD primers

| Primer | | | SEQ ID |
|---|---|---|---|
| No. | Name | sequence | NO. |
| 1 | RBS-AcoD-f | CGAGGAAAGAGGAGAAAGAA | 95 |
| | | GCTTATGAATATGGCAGAAAT | |
| | | TGCCCAG | |
| 2 | AcoD-ter-r | ATCTACACTAGCACTATCGAG | 96 |
| | | CTCTTAAAAAAAGCCCAGGG | |
| | | CATTCGG | |
| 3 | AcoD-ter-f | CCGAATGCCCTGGGCTTTTTT | 97 |
| | | TAAGAGCTCGATAGTGCTAGT | |
| | | GTAGAT | |
| 4 | kefB RH-r | ACTCAAATTCATCCCAGCCGT | 98 |
| | | CCAGCTG | |
| 5 | kefB LH-f | TTGTTTATGGATGCGCTGGGG | 99 |
| | | TTGTCGATGG | |
| 6 | RBS-AcoD-r | CTGGGCAATTTCTGCCATATT | 100 |
| | | CATAAGCTTCTTTCTCCTCTT | |
| | | TCCTCG | |

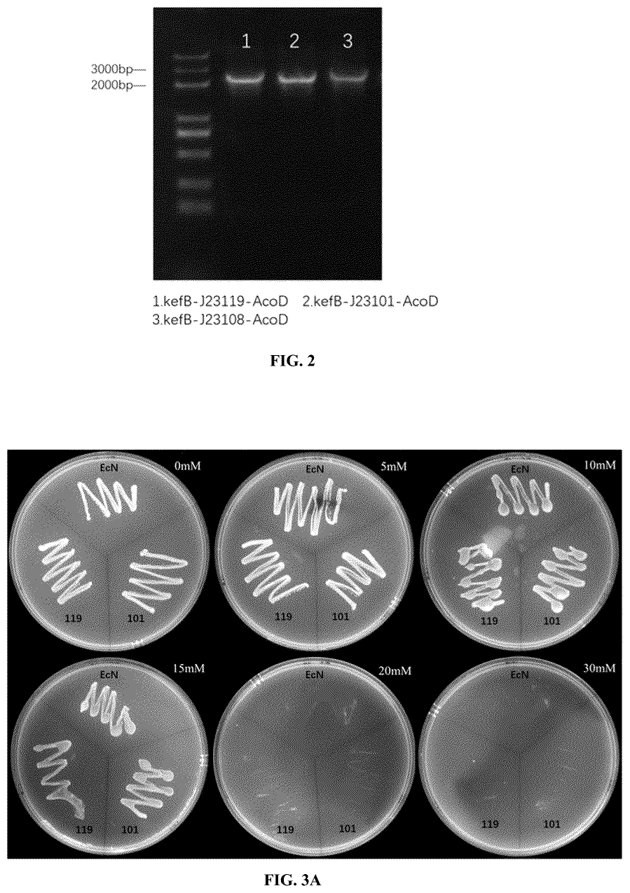

The kefB-J23119-AcoD, kefB-J23101-AcoD and kefB-J23108-AcoD fragments were confirmed to be correct in size (2489 bp) as shown in FIG. 2, and can be used for further genome integration in next step.

(amino acid sequence of AcoD from Cupriavidus
necator)
SEQ ID NO: 1
MNMAEIAQLGVSNPYKQQYENYIGGAWVPPAGGEYFESTTPITGKPFTRV
PRSGQQDVDAALDAAHAAKAAWARTSTTERANILNRIADRIEANLKLLAV
AESIDNGKPVRETTAADLPLAVDHFRYFAGCIRAQEGGISEIDADTIAYH
FHEPLGVVGQIIPWNFPLLMATWKLAPALAAGNCVVLKPAEQTPASILVL
MEVIGDLLPPGVVNVINGFGLEAGKPLASSPRISKVAFTGETTTGRLIMQ
YASQNLIPVTLELGGKSPNIFFEDVLAADDAFFDKALEGFAMFALNQGEV
CTCPSRALIQESIYDREMERALKRVAAIRQGHPLDTGTMIGAQASAEQLE
KILSYIDLGRKEGAQCLIGGERNVLDGDLAGGYYVKPTVFAGHNKMRIFQ
EEIFGPVVSVTTFKDEEEALAIANDTLYGLGAGVWIRDGARAFRMGRGIQ
AGRVWTNCYHAYPAHAAFGGYKqSGIGRENHRMMLDHYQQTKNLLVSYSP
NALGFF
(pUC57-AcoD plasmid)
SEQ ID NO: 2
TCGCGCGTTTCGGTGATGACGGTGAAAACCTCTGACACATGCAGCTCCCG
GAGACGGTCACAGCTTGTCTGTAAGCGGATGCCGGGAGCAGACAAGCCCG
TCAGGGCGCGTCAGCGGGTGTTGGCGGGTGTCGGGGCTGGCTTAACTATG
CGGCATCAGAGCAGATTGTACTGAGAGTGCACCATATGCGGTGTGAAATA
CCGCACAGATGCGTAAGGAGAAAATACCGCATCAGGCGCCATTCGCCATT
CAGGCTGCGCAACTGTTGGGAAGGGCGATCGGTGCGGGCCTCTTCGCTAT
TACGCCAGCTGGCGAAAGGGGGATGTGCTGCAAGGCGATTAAGTTGGGTA
ACGCCAGGGTTTTCCCAGTCACGACGTTGTAAAACGACGGCCAGTGAATT
CGAGCTCGGTACCTCGCGAATGCATCTAGATATGAATATGGCAGAAATTG
CCCAGCTGGGTGTGAGTAATCCGTATAAACAGCAGTATGAAAATTATATT
GGTGGTGCATGGGTTCCGCCAGCTGGCGGTGAATATTTTGAATCAACCAC
CCCGATTACCGGCAAACCGTTTACCCGTGTTCCGCGTAGCGGTCAGCAGG
ATGTGGATGCCGCACTGGATGCAGCACATGCAGCCAAAGCCGCATGGGCA
CGTACCTCTACCACCGAACGTGCCAATATTCTGAATCGCATTGCCGATCG
CATTGAAGCCAATCTGAAACTGCTGGCAGTTGCCGAATCTATTGATAATG
GTAAACCGGTTCGTGAAACCACCGCCGCCGATCTGCCGTTAGCAGTGGAT
CATTTTCGCTATTTTGCAGGTTGTATTCGCGCCCAGGAAGGCGGCATTAG
CGAAATTGATGCAGATACCATTGCATATCATTTTCATGAACCGTTAGGCG
TTGTGGGCCAGATTATTCCGTGGAATTTTCCGCTGTTAATGGCAACCTGG
AAACTGGCCCCGGCCTTAGCAGCAGGTAATTGTGTTGTGCTGAAACCCGC
CGAACAGACCCCGGCCTCAATTCTGGTGTTAATGGAAGTGATTGGCGATT
TACTGCCGCCGGGCGTTGTTAATGTGATTAATGGCTTTGGCTTAGAAGCA
GGTAAACCGCTGGCAAGCTCTCCGCGCATTTCTAAAGTTGCCTTTACCGG
CGAAACCACCACCGGTCGTCTGATTATGCAGTATGCAAGTCAGAATCTGA
TTCCGGTGACCTTAGAACTGGGTGGTAAAAGTCCGAATATTTTTTTTGAA
GATGTGCTGGCCGCCGATGATGCCTTTTTTGATAAAGCCCTGGAAGGCTT
TGCCATGTTTGCACTGAATCAGGGCGAAGTTTGTACCTGTCCGTCACGCG
CACTGATTCAGGAATCAATTTATGATCGCTTTATGGAACGCGCCTTAAAA
CGGGTTGCAGCAATTCGTCAGGGCCATCCGTTAGATACCGGTACCATGAT
TGGCGCACAGGCCTCTGCCGAACAGTTAGAAAAAATTCTGAGCTATATTG
ATCTGGGTCGCAAAGAAGGCGCCCAGTGTCTGACCGGCGGTGAACGTAAT
GTGCTGGATGGCGATTTAGCCGGTGGCTATTATGTTAAACCGACCGTGTT
TGCAGGTCATAATAAAATGCGCATTTTTCAGGAAGAAATTTTTGGTCCGG
TTGTGAGCGTGACCACCTTTAAAGATGAAGAAGAAGCCTTAGCCATTGCC
AATGATACCCTGTATGGTTTAGGTGCAGGCGTGTGGACCCGCGATGGTGC
ACGCGCCTTTCGTATGGGTCGTGGTATTCAGGCAGGTCGCGTTTGGACCA
ATTGTTATCATGCCTATCCGGCACATGCAGCCTTTGGCGGCTATAAACAG
AGCGGTATTGGTCGCGAAAATCATCGTATGATGTTAGATCATTATCAGCA
GACCAAAAATCTGTTAGTGTCTTATAGTCCGAATGCCCTGGGCTTTTTTT
AAGGATCCCGGGCCCGTCGACTGCAGAGGCCTGCATGCAAGCTTGGCGTA
ATCATGGTCATAGCTGTTTCCTGTGTGAAATTGTTATCCGCTCACAATTC
CACACAACATACGAGCCGGAAGCATAAAGTGTAAAGCCTGGGGTGCCTAA
TGAGTGAGCTAACTCACATTAATTGCGTTGCGCTCACTGCCCGCTTTCCA
GTCGGGAAACCTGTCGTGCCAGCTGCATTAATGAATCGGCCAACGCGCGG
GGAGAGGCGGTTTGCGTATTGGGCGCTCTTCCGCTTCCTCGCTCACTGAC
TCGCTGCGCTCGGTCGTTCGGCTGCGGCGAGCGGTATCAGCTCACTCAAA
GGCGGTAATACGGTTATCCACAGAATCAGGGGATAACGCAGGAAAGAACA
TGTGAGCAAAAGGCCAGCAAAAGGCCAGGAACCGTAAAAAGGCCGCGTTG
CTGGCGTTTTTCCATAGGCTCCGCCCCCCTGACGAGCATCACAAAAATCG
ACGCTCAAGTCAGAGGTGGCGAAACCCGACAGGACTATAAAGATACCAGG
CGTTTCCCCCTGGAAGCTCCCTCGTGCGCTCTCCTGTTCCGACCCTGCCG
CTTACCGGATACCTGTCCGCCTTTCTCCCTTCGGGAAGCGTGGCGCTTTC
TCATAGCTCACGCTGTAGGTATCTCAGTTCGGTGTAGGTCGTTCGCTCCA
AGCTGGGCTGTGTGCACGAACCCCCCGTTCAGCCCGACCGCTGCGCCTTA
TCCGGTAACTATCGTCTTGAGTCCAACCCGGTAAGACACGACTTATCGCC
ACTGGCAGCAGCCACTGGTAACAGGATTAGCAGAGCGAGGTATGTAGGCG
GTGCTACAGAGTTCTTGAAGTGGTGGCCTAACTACGGCTACACTAGAAGA
ACAGTATTTGGTATCTGCGCTCTGCTGAAGCCAGTTACCTTCGGAAAAAG
AGTTGGTAGCTCTTGATCCGGCAAACAAACCACCGCTGGTAGCGGTGGTT
TTTTTGTTTGCAAGCAGCAGATTACGCGCAGAAAAAAAGGATCTCAAGAA
GATCCTTTGATCTTTTCTACGGGGTCTGACGCTCAGTGGAACGAAAACTC
ACGTTAAGGGATTTTGGTCATGAGATTATCAAAAAGGATCTTCACCTAGA
TCCTTTTAAATTAAAAATGAAGTTTTAAATCAATCTAAAGTATATATGAG
TAAACTTGGTCTGACAGTTACCAATGCTTAATCAGTGAGGCACCTATCTC
AGCGATCTGTCTATTTCGTTCATCCATAGTTGCCTGACTCCCCGTCGTGT
AGATAACTACGATACGGGAGGGCTTACCATCTGGCCCCAGTGCTGCAATG
ATACCGCGAGACCCACGCTCACCGGCTCCAGATTTATCAGCAATAAACCA
GCCAGCCGGAAGGGCCGAGCGCAGAAGTGGTCCTGCAACTTTATCCGCCT
CCATCCAGTCTATTAATTGTTGCCGGGAAGCTAGAGTAAGTAGTTCGCCA
GTTAATAGTTTGCGCAACGTTGTTGCCATTGCTACAGGCATCGTGGTGTC
ACGCTCGTCGTTTGGTATGGCTTCATTCAGCTCCGGTTCCCAACGATCAA
GGCGAGTTACATGATCCCCCATGTTGTGCAAAAAAGCGGTTAGCTCCTTC
GGTCCTCCGATCGTTGTCAGAAGTAAGTTGGCCGCAGTGTTATCACTCAT
GGTTATGGCAGCACTGCATAATTCTCTTACTGTCATGCCATCCGTAAGAT
GCTTTTCTGTGACTGGTGAGTACTCAACCAAGTCATTCTGAGAATAGTGT
ATGCGGCGACCGAGTTGCTCTTGCCCGGCGTCAATACGGGATAATACCGC
GCCACATAGCAGAACTTTAAAAGTGCTCATCATTGGAAAACGTTCTTCGG
GGCGAAAACTCTCAAGGATCTTACCGCTGTTGAGATCCAGTTCGATGTAA
CCCACTCGTGCACCCAACTGATCTTCAGCATCTTTTACTTTCACCAGCGT
TTCTGGGTGAGCAAAAACAGGAAGGCAAAATGCCGCAAAAAAGGGAATAA
GGGCGACACGGAAATGTTGAATACTCATACTCTTCCTTTTTCAATATTAT
TGAAGCATTTATCAGGGTTATTGTCTCATGAGCGGATACATATTTGAATG
TATTTAGAAAAATAAACAAATAGGGGTTCCGCGCACATTTCCCCGAAAAG
TGCCACCTGACGTCTAAGAAACCATTATTATCATGACATTAACCTATAAA
AATAGGCGTATCACGAGGCCCTTTCGTC
(codon-optimized AcoD)
SEQ ID NO: 111
ATGAATATGGCAGAAATTGCCCAGCTGGGTGTGAGTAATCCGTATAAACA
GCAGTATGAAAATTATATTGGTGGTGCATGGGTTCCGCCAGCTGGCGGTG
AATATTTTGAATCAACCACCCCGATTACCGGCAAACCGTTTACCCGTGTT
CCGCGTAGCGGTCAGCAGGATGTGGATGCCGCACTGGATGCAGCACATGC
AGCCAAAGCCGCATGGGCACGTACCTCTACCACCGAACGTGCCAATATTC
TGAATCGCATTGCCGATCGCATTGAAGCCAATCTGAAACTGCTGGCAGTT
GCCGAATCTATTGATAATGGTAAACCGGTTCGTGAAACCACCGCCGCCGA
TCTGCCGTTAGCAGTGGATCATTTTCGCTATTTTGCAGGTTGTATTCGCG
CCCAGGAAGGCGGCATTAGCGAAATTGATGCAGATACCATTGCATATCAT
TTTCATGAACCGTTAGGCGTTGTGGGCCAGATTATTCCGTGGAATTTTCC
GCTGTTAATGGCAACCTGGAAACTGGCCCCGGCCTTAGCAGCAGGTAATT
GTGTTGTGCTGAAACCCGCCGAACAGACCCCGGCCTCAATTCTGGTGTTA
ATGGAAGTGATTGGCGATTTACTGCCGCCGGGCGTTGTTAATGTGATTAA
TGGCTTTGGCTTAGAAGCAGGTAAACCGCTGGCAAGCTCTCCGCGCATTT
CTAAAGTTGCCTTTACCGGCGAAACCACCACCGGTCGTCTGATTATGCAG
TATGCAAGTCAGAATCTGATTCCGGTGACCTTAGAACTGGGTGGTAAAAG
TCCGAATATTTTTTTTGAAGATGTGCTGGCCGCCGATGATGCCTTTTTTG
ATAAAGCCCTGGAAGGCTTTGCCATGTTTGCACTGAATCAGGGCGAAGTT
TGTACCTGTCCGTCACGCGCACTGATTCAGGAATCAATTTATGATCGCTT
TATGGAACGCGCCTTAAAACGGGTTGCAGCAATTCGTCAGGGCCATCCGT
TAGATACCGGTACCATGATTGGCGCACAGGCCTCTGCCGAACAGTTAGAA
AAAATTCTGAGCTATATTGATCTGGGTCGCAAAGAAGGCGCCCAGTGTCT
GACCGGCGGTGAACGTAATGTGCTGGATGGCGATTTAGCCGGTGGCTATT
ATGTTAAACCGACCGTGTTTGCAGGTCATAATAAAATGCGCATTTTTCAG
GAAGAAATTTTTGGTCCGGTTGTGAGCGTGACCACCTTTAAAGATGAAGA
AGAAGCCTTAGCCATTGCCAATGATACCCTGTATGGTTTAGGTGCAGGCG
TGTGGACCCGCGATGGTGCACGCGCCTTTCGTATGGGTCGTGGTATTCAG
GCAGGTCGCGTTTGGACCAATTGTTATCATGCCTATCCGGCACATGCAGC
CTTTGGCGGCTATAAACAGAGCGGTATTGGTCGCGAAAATCATCGTATGA
TGTTAGATCATTATCAGCAGACCAAAAATCTGTTAGTGTCTTATAGTCCG
AATGCCCTGGGCTTTTTTTAA

### Example 3. Construction of Genetically Engineered EcN

Electroporation-competent EcN cells were prepared. 200 ng of the gRNA cutting plasmid ZL-003_kefB as described in Example 1 and 2 μg of the donor RNA fragment for homologous recombination as described in Example 2 were added into 100 μL of the electroporation-competent cells. The cells were transferred to a pre-cooled 2-mm electroporation cuvette after mixture, and electroporated at the condition listed in Table 4. After transformation, cells were recovered in 900 μL SOC culture medium at 30° C. and incubated in 220 RPM for 3 hours. After this, the cells were plated on LB agar plates supplemented with 50 μg/mL spectinomycin, 50 μg/mL streptomycin and 100 μg/mL ampicillin and incubated at 30° C. overnight.

TABLE 4
Electroporation Condition

| Parameter | Condition |
|---|---|
| Voltage (V) | 2500 |
| Capacitance (μF) | 25 |
| Resistance (Ω) | 200 |
| Electroporation Cuvette (mm) | 2 |

### 1. PCR Validation

8 single colonies were picked from each of the three plates: EcN/kefB::J23119-AcoD, EcN/kefB::J23101-AcoD, and EcN/kefB::J23108-AcoD, and resuspended in 30 μL LB media for PCR verification. Primers kefB-verify-f: GCAGACGAACATTTCGACTG (SEQ ID NO: 101) and kefB-verify-r:ATCGCCATTGAATCCTGTGC (SEQ ID NO: 102) were used to carry out the PCR verification, with 1 μL bacterial suspension as a template (2×rapid Taq Master Mix).The PCR reaction system is shown in Table 5.

TABLE 5
PCR Reaction System of 2× Rapid Taq Master Mix (50 μL)

| Reagent | Volume |
|---|---|
| 2× Rapid Taq Master Mix | 25 μL |
| Primer 1(10 μM) | 1 μL |
| Primer 2(10 μM) | 1 μL |
| Bacterial Suspension | 1 μL |
| H₂O | Supplemented to 50 μg |

PCR reaction conditions are listed in Table 6.

TABLE 6
PCR Process of Taq

| Step | Temperature | Time | | Process |
|---|---|---|---|---|
| Predenaturing | 98° C. | 5 | min | (Denaturing, |
| Denaturing | 98° C. | 15 | s | Annealing |
| Annealing | (Tm − 2)° C. | 15 | s | Extending) |

| | | | |
|---|---|---|---|
| Extending | 68° C. | 1 kb/15 s | for 35 cycles |
| Preservation | 16° C. | — | |

The PCR products of the strains that exhibited consistent with the target band in the electrophoresis results were further sent for sequencing (Shanghai Sunny Biotechnology Co., Ltd.). The strains with correct genome integration from the sequencing results were selected as the genetically engineered strains, including EcN/kefB:J23119-AcoD (engineered bacterial 119), EcN/kefB::J23101-AcoD (engineered bacterial 101) and EcN/kefB::J23108-AcoD (engineered bacterial 108).

2. Construction of Chaperon Plasmids

The chaperone plasmid pKJE7 (expressing molecular chaperones: dnaK, dnaJ and grpE) and pGro-TF2 (expressing molecular chaperones: groES, groEL and tig) purchased from Takara Biomedical Technology (Beijing) Co., Ltd. were used for molecular chaperone genes amplification.

The primers grpE-ter-f: ACTGTAGCGAAAGCAAAAGCTTAATAACGCTGATAGTGCTAGTGTAGATCGC (SEQ ID NO: 103) and RBS-dnaK-r: AGGTCGATACCAATTATTTTACCCATTGAGACCTTTCTCCTCTTTCCTCG (SEQ ID NO: 104) were used to amplify the ZL-003_lldD skeleton with ZL-003_lldD as a template (preserved in our lab) using a high fidelity DNA polymeras KOD. Simultaneously, the primers RBS-dnaK-f: GAAAGAGGAGAAAGGTCTCAATGGGTAAAATAATTGGTATCGACCT (SEQ ID NO: 105) and grpE-ter-r: CACTAGCACTATCAGCGTTATTAAGCTTTTGCTTTCGCTACAGT (SEQ ID NO: 106) were used to amplify the dnaK-dnaJ-grpE fragment with the plasmid Pkje7 as a template using the high fidelity DNA polymeras KOD. The ZL-003_lldD skeleton and the dnaK-dnaJ-grpE fragment were ligated using the Clon Express Ultra One Step Cloning Kit to obtain the integrative plasmid ZL-003_IldD-J23115-KJE (SEQ ID NO: 8).

The primers tig-ter-f: CTGATGAACCAGCAGGCGTAATAACGCTGATAGTGCTAGTGTAGATCGC (SEQ ID NO: 107) and RBS-groES-r: CGATCATGCAATGGACGAATATTCATTGAGACCTTTCTCCTCTTTCCTC (SEQ ID NO: 108) were used to amplify the ZL-003_tkrA skeleton with the ZL-003_tkrA as a template using the high fidelity DNA polymeras KOD. Simultaneously, the primers RBS-groES-f: GAAAGAGGAGAAAGGTCTCAATGAATATTCGTCCATTGCATGATCG (SEQ ID NO: 109) and tig-ter-r: CACTAGCACTATCAGCGTTATTACGCCTGCTGGTTCATCAG (SEQ ID NO: 110) were used to amplify the groES-groEL-tig fragment using the KOD DNA polymerase with the plasmid pG-TF2 as a template. The ZL-003_tkrA skeleton and the groES-groEL-tig fragment were ligated using the Clon Express Ultra One Step Cloning Kit to obtain the integrative plasmid ZL-003_tkrA-J23115-Gro (SEQ ID NO: 9).

The 119-4 (EcN/kefB::J23119-AcoD-4) competent cells were prepared and then transformed with the plasmids ZL-003_IldD-J23115-KJE and ZL-003_tkrA-J23115-Gro respectively. The target strains 119-4/IldD::J23115-KJE and 119-4/tkrA::J23115-Gro were obtained after PCR verification and sequencing.

The plasmids and strains used in the present invention are listed in Table 7 and Table 8 respectively.

TABLE 7
The plasmids and DNA fragments used in this research

| | | SEQ ID | |
|---|---|---|---|
| Name | Function | Number | Origin |
| ZL-003_kefB | kefB Gene Cleavage Plasmid | SEQ ID NO: 6 | Stored in our laboratory |
| pUC57-AcoD | AcoD Gene Template | SEQ ID NO: 2 | Synthesized by Shanghai |
| | | | Sunny Biotechnology Co., Ltd. |
| kefB-J23119-AcoD | Integrative Fragment | SEQ ID NO: 7 | Constructed by this research |
| | (J23119-AcoD integrated | | |
| | into site kefB) | | |
| ZL-003_IldD-J23115-KJE | Integrative Plasmid | SEQ ID NO: 8 | Constructed by this research |
| | (dnaK-dnaJ-grpE gene | | |
| | integrated into site IldD) | | |
| ZL-003_tkrA-J23115-Gro | Integrative Plasmid | SEQ ID NO: 9 | Constructed by this research |
| | (groES-groEL-tig gene | | |
| | integrated into of site tkrA) | | |

TABLE 8
The strains used in this research

| Name of | | Chassis | |
|---|---|---|---|
| Strain | Genotype | Bacterial | Origin |
| Control | E. coli nissle 1917 (EcN) | | China General |
| Bacterial | | | Microbiological Culture |
| | | | Collection Center |
| Engineered | EcN/kefB::J23119-AcoD | EcN | Constructed by this |
| Bacterial 119 | | | research |
| Engineered | EcN/kefB::J23101-AcoD | EcN | Constructed by this |
| Bacterial 101 | | | research |
| Engineered | EcN/kefB::J23108-AcoD | EcN | Constructed by this |
| Bacterial 108 | | | research |
| KJE | EcN/kefB::J23119-AcoD/IldD::J23115-dnaK-dnaJ-grpE | Engineered | Constructed by this |
| | | Bacterial | research |
| | | 119 | |
| Gro | EcN/kefB::J23119-AcoD/tkrA::groES-groEL-tig | Engineered | Constructed by this |
| | | Bacterial | research |
| | | 119 | |

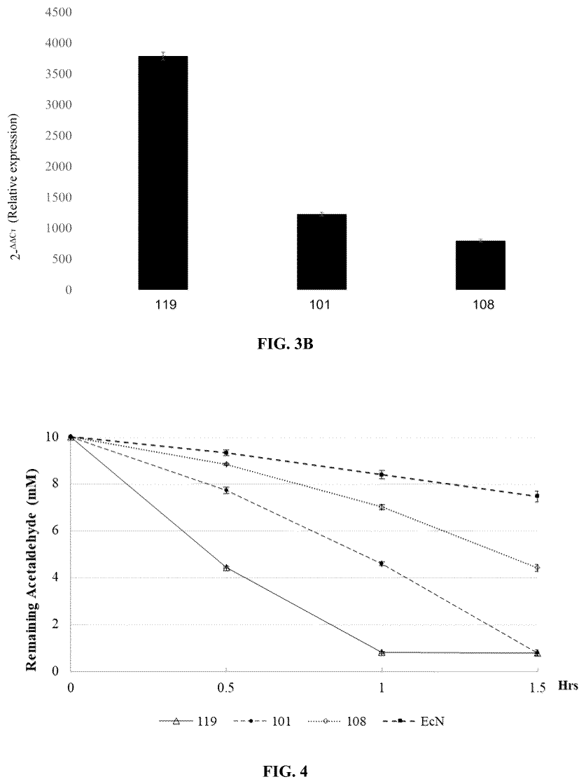

FIG. 3B shows that the transcriptional level of AcoD gene in engineered bacteria 119 was three times of that in engineered bacteria 101, and was six times of that in engineered bacteria 108. This, in combination with the result as shown in FIG. 3A, suggests that the promoter J23119 in engineered bacteria 119 is preferred in the present invention.

SEQ ID NO: 3

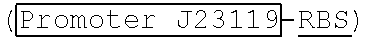

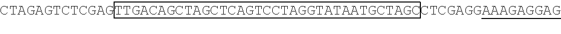

AAAG
SEQ ID NO: 4

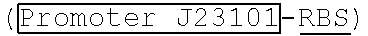

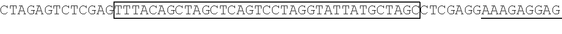

AAAG
SEQ ID NO: 5

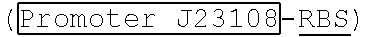

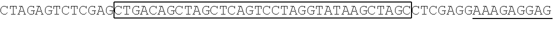

AAAG
SEQ ID NO: 6

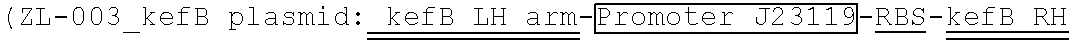

arm)
CCACCTGACGTCTAAGAAACCATTATTATCATGACATTAACCTATAAAAATAGGCGTATCACGA
GGCAGAATTTCAGATAAAAAAAATCCTTAGCTTTCGCTAAGGATGATTTCTGGAATTCGCGGCC
GCATTTGTTTATGGATGCGCTGGGGTTGTCGATGGCGCTCGGTACGTTTATTGCGGGTGTGCTA
CTGGCGGAAAGTGAATATCGCCATGAACTGGAAACGGCTATCGATCCCTTCAAAGGCTTGCTGC
TCGGTTTGTTCTTTATCTCTGTCGGCATGTCGCTCAACCTCGGGGTGCTTTATACCCATCTGTT
GTGGGTAGTGATAAGTGTGGTTGTGCTGGTGGCGGTGAAAATTCTCGTGCTGTATCTGCTGGCG
CGATTGTATGGCGTGCGCAGTTCTGAGCGGATGCAGTTTGCTGGCGTGTTGAGTCAGGGCGGTG
AGTTTGCCTTTGTCCTCTTTTCTACCGCTTCTTCACAACGCTTATTCCAGGGCGACCAGATTCT

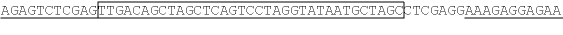

AGGTCTCAATGCGTAAAGGCGAAGAGCTGTTCACTGGTGTCGTCCCTATTCTGGTGGAACTGGA
TGGTGATGTCAACGGTCATAAGTTTTCCGTGCGTGGCGAGGGTGAAGGTGACGCAACTAATGGT
AAACTGACGCTGAAGTTCATCTGTACTACTGGTAAACTGCCGGTACCTTGGCCGACTCTGGTAA
CGACGCTGACTTATGGTGTTCAGTGCTTTGCTCGTTATCCGGACCATATGAAGCAGCATGACTT
CTTCAAGTCCGCCATGCCGGAAGGCTATGTGCAGGAACGCACGATTTCCTTTAAGGATGACGGC
ACGTACAAAACGCGTGCGGAAGTGAAATTTGAAGGCGATACCCTGGTAAACCGCATTGAGCTGA
AAGGCATTGACTTTAAAGAAGACGGCAATATCCTGGGCCATAAGCTGGAATACAATTTTAACAG
CCACAATGTTTACATCACCGCCGATAAACAAAAAAATGGCATTAAAGCGAATTTTAAAATTCGC
CACAACGTGGAGGATGGCAGCGTGCAGCTGGCTGATCACTACCAGCAAAACACTCCAATCGGTG
ATGGTCCTGTTCTGCTGCCAGACAATCACTATCTGAGCACGCAAAGCGTTCTGTCTAAAGATCC
GAACGAGAAACGCGATCATATGGTTCTGCTGGAGTTCGTAACCGCAGCGGGCATCGCGCATGGT
ATGGATGAACTGTACAGATGATAACGCTGATAGTGCTAGTGTAGATCGCTACTAGAGCCAGGCA
TCAAATAAAACGAAAGGCTCAGTCGAAAGACTGGGCCTTTCGTTTTATCTGTTGTTTGTCGGTG
AACGCTCTCTACTAGAGTCACACTGGCTCACCTTCGGGTGGGCCTTTCTGCGTTTATATACTAG
AAGCGGCCGCTGCAGTTGCATATTCTTGCGCGAGCGCGCGGACGTGTGGAAGCGCATGAGTTAT
TACAGGCAGGGGTGACGCAGTTTTCCCGTGAAACATTCTCCAGTGCGTTAGAGCTGGGGCGCAA
GACGCTGGTCACGCTTGGCATGCATCCGCATCAGGCGCAGCGCGCGCAACTGCATTTTCGCCGC
CTGGATATGCGAATGCTGCGGGAGTTAATCCCGATGCATGCTGATACCGTACAAATTTCTCGCG
CCAGGGAAGCCCGACGTGAACTGGAAGAGATTTTCCAGCGTGAAATGCAACAAGAACGACGCCA
GCTGGACGGCTGGGATGAATTTGAGTTGCAGGAATTCAAAAAAAGCACCGACTCGGTGCCACTT
TTTCAAGTTGATAACGGACTAGCCTTATTTTAACTTGCTATTTCTAGCTCTAAAACGCTTCATA
GTGTCTTCCGGCACTAGTATTATACCTAGGACTGAGCTAGCTGTCAAGGATCCAGCATATGCGG
TGTGAAATACCGCACAGATGCGTAAGGAGAAAATACCGCATCAGGCGCTCTTCCGCTTCCTCGC
TCACTGACTCGCTGCGCTCGGTCGTTCGGCTGCGGCGAGCGGTATCAGCTCACTCAAAGGCGGT
AATACGGTTATCCACAGAATCAGGGGATAACGCAGGAAAGAACATGTGAGCAAAAGGCCAGCAA
AAGGCCAGGAACCGTAAAAAGGCCGCGTTGCTGGCGTTTTTCCATAGGCTCCGCCCCCCTGACG
AGCATCACAAAAATCGACGCTCAAGTCAGAGGTGGCGAAACCCGACAGGACTATAAAGATACCA
GGCGTTTCCCCCTGGAAGCTCCCTCGTGCGCTCTCCTGTTCCGACCCTGCCGCTTACCGGATAC
CTGTCCGCCTTTCTCCCTTCGGGAAGCGTGGCGCTTTCTCATAGCTCACGCTGTAGGTATCTCA
GTTCGGTGTAGGTCGTTCGCTCCAAGCTGGGCTGTGTGCACGAACCCCCCGTTCAGCCCGACCG
CTGCGCCTTATCCGGTAACTATCGTCTTGAGTCCAACCCGGTAAGACACGACTTATCGCCACTG
GCAGCAGCCACTGGTAACAGGATTAGCAGAGCGAGGTATGTAGGCGGTGCTACAGAGTTCTTGA
AGTGGTGGCCTAACTACGGCTACACTAGAAGGACAGTATTTGGTATCTGCGCTCTGCTGAAGCC
AGTTACCTTCGGAAAAAGAGTTGGTAGCTCTTGATCCGGCAAACAAACCACCGCTGGTAGCGGT
GGTTTTTTTGTTTGCAAGCAGCAGATTACGCGCAGAAAAAAAGGATCTCAAGAAGATCCTTTGA
TCTTTTCTACGGGGTCTGACGCTCAGTGGAACGAAAACTCACGTTAAGGGATTTTGGTCATGAG
ATTATCAAAAAGGATCTTCACCTAGATCCTTTTAAATTAAAAATGAAGTTTTAAATCAATCTAA
AGTATATATGAGTAAACTTGGTCTGACAGTTACCAATGCITAATCAGTGAGGCACCTATCTCAG
CGATCTGTCTATTTCGTTCATCCATAGTTGCCTGACTCCCCGTCGTGTAGATAACTACGATACG
GGAGGGCTTACCATCTGGCCCCAGTGCTGCAATGATACCGCGTGACCCACGCTCACCGGCTCCA
GATTTATCAGCAATAAACCAGCCAGCCGGAAGGGCCGAGCGCAGAAGTGGTCCTGCAACTTTAT
CCGCCTCCATCCAGTCTATTAATTGTTGCCGGGAAGCTAGAGTAAGTAGTTCGCCAGTTAATAG
TTTGCGCAACGTTGTTGCCATTGCTACAGGCATCGTGGTGTCACGCTCGTCGTTTGGTATGGCT
TCATTCAGCTCCGGTTCCCAACGATCAAGGCGAGTTACATGATCCCCCATGTTGTGCAAAAAAG
CGGTTAGCTCCTTCGGTCCTCCGATCGTTGTCAGAAGTAAGTTGGCCGCAGTGTTATCACTCAT
GGTTATGGCAGCACTGCATAATTCTCTTACTGTCATGCCATCCGTAAGATGCTTTTCTGTGACT
GGTGAGTACTCAACCAAGTCATTCTGAGAATAGTGTATGCGGCGACCGAGTTGCTCTTGCCCGG
CGTCAATACGGGATAATACCGCGCCACATAGCAGAACTTTAAAAGTGCTCATCATTGGAAAACG
TTCTTCGGGGCGAAAACTCTCAAGGATCTTACCGCTGTTGAGATCCAGTTCGATGTAACCCACT
CGTGCACCCAACTGATCTTCAGCATCTTTTACTTTCACCAGCGTTTCTGGGTGAGCAAAAACAG
GAAGGCAAAATGCCGCAAAAAAGGGAATAAGGGCGACACGGAAATGTTGAATACTCATACTCTT
CCTTTTTCAATATTATTGAAGCATTTATCAGGGTTATTGTCTCATGAGCGGATACATATTTGAA
TGTATTTAGAAAAATAAACAAATAGGGGTTCCGCGCACATTTCCCCGAAAAGTG
SEQ ID NO: 7

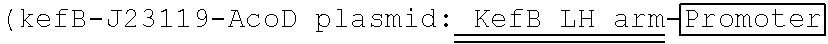

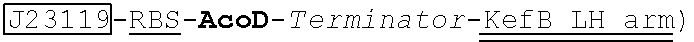

TTGTTTATGGATGCGCTGGGGTTGTCGATGGCGCTCGGTACGTTTATTGCGGGTGTGCTACTGG
CGGAAAGTGAATATCGCCATGAACTGGAAACGGCTATCGATCCCTTCAAAGGCTTGCTGCTCGG
TTTGTTCTTTATCTCTGTCGGCATGTCGCTCAACCTCGGGGTGCTTTATACCCATCTGTTGTGG
GTAGTGATAAGTGTGGTTGTGCTGGTGGCGGTGAAAATTCTCGTGCTGTATCTGCTGGCGCGAT
TGTATGGCGTGCGCAGTTCTGAGCGGATGCAGTTTGCTGGCGTGTTGAGTCAGGGCGGTGAGTT
TGCCTTTGTCCTCTTTTCTACCGCTTCTTCACAACGCTTATTCCAGGGCGACCAGATTCTAGAG

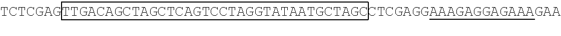

GCTTATGAATATGGCAGAAATTGCCCAGCTGGGTGTGAGTAATCCGTATAAACAGCAGTATGAA
AATTATATTGGTGGTGCATGGGTTCCGCCAGCTGGCGGTGAATATTTTGAATCAACCACCCCGA
TTACCGGCAAACCGTTTACCCGTGTTCCGCGTAGCGGTCAGCAGGATGTGGATGCCGCACTGGA
TGCAGCACATGCAGCCAAAGCCGCATGGGCACGTACCTCTACCACCGAACGTGCCAATATTCTG
AATCGCATTGCCGATCGCATTGAAGCCAATCTGAAACTGCTGGCAGTTGCCGAATCTATTGATA
ATGGTAAACCGGTTCGTGAAACCACCGCCGCCGATCTGCCGTTAGCAGTGGATCATTTTCGCTA
TTTTGCAGGTTGTATTCGCGCCCAGGAAGGCGGCATTAGCGAAATTGATGCAGATACCATTGCA
TATCATTTTCATGAACCGTTAGGCGTTGTGGGCCAGATTATTCCGTGGAATTTTCCGCTGTTAA
TGGCAACCTGGAAACTGGCCCCGGCCTTAGCAGCAGGTAATTGTGTTGTGCTGAAACCCGCCGA
ACAGACCCCGGCCTCAATTCTGGTGTTAATGGAAGTGATTGGCGATTTACTGCCGCCGGGCGTT
GTTAATGTGATTAATGGCTTTGGCTTAGAAGCAGGTAAACCGCTGGCAAGCTCTCCGCGCATTT
CTAAAGTTGCCTTTACCGGCGAAACCACCACCGGTCGTCTGATTATGCAGTATGCAAGTCAGAA
TCTGATTCCGGTGACCTTAGAACTGGGTGGTAAAAGTCCGAATATTTTTTTTGAAGATGTGCTG
GCCGCCGATGATGCCTTTTTTGATAAAGCCCTGGAAGGCTTTGCCATGTTTGCACTGAATCAGG
GCGAAGTTTGTACCTGTCCGTCACGCGCACTGATTCAGGAATCAATTTATGATCGCTTTATGGA
ACGCGCCTTAAAACGGGTTGCAGCAATTCGTCAGGGCCATCCGTTAGATACCGGTACCATGATT
GGCGCACAGGCCTCTGCCGAACAGTTAGAAAAAATTCTGAGCTATATTGATCTGGGTCGCAAAG
AAGGCGCCCAGTGTCTGACCGGCGGTGAACGTAATGTGCTGGATGGCGATTTAGCCGGTGGCTA
TTATGTTAAACCGACCGTGTTTGCAGGTCATAATAAAATGCGCATTTTTCAGGAAGAAATTTTT
GGTCCGGTTGTGAGCGTGACCACCTTTAAAGATGAAGAAGAAGCCTTAGCCATTGCCAATGATA
CCCTGTATGGTTTAGGTGCAGGCGTGTGGACCCGCGATGGTGCACGCGCCTTTCGTATGGGTCG
TGGTATTCAGGCAGGTCGCGTTTGGACCAATTGTTATCATGCCTATCCGGCACATGCAGCCTTT
GGCGGCTATAAACAGAGCGGTATTGGTCGCGAAAATCATCGTATGATGTTAGATCATTATCAGC
AGACCAAAAATCTGTTAGTGTCTTATAGTCCGAATGCCCTGGGCTTTTTTTAAGAGCTCGATAG
TGCTAGTGTAGATCGCTACTAGAGCCAGGCATCAAATAAAACGAAAGGCTCAGTCGAAAGACTG
GGCCTTTCGTTTTATCTGTTGTTTGTCGGTGAACGCTCTCTACTAGAGTCACACTGGCTCACCT
TCGGGTGGGCCTTTCTGCGTTTATATACTAGAAGCGGCCGCTGCAGTTGCATATTCTTGCGCGA
GCGCGCGGACGTGTGGAAGCGCATGAGTTATTACAGGCAGGGGTGACGCAGTTTTCCCGTGAAA
CATTCTCCAGTGCGTTAGAGCTGGGGCGCAAGACGCTGGTCACGCTTGGCATGCATCCGCATCA
GGCGCAGCGCGCGCAACTGCATTTTCGCCGCCTGGATATGCGAATGCTGCGGGAGTTAATCCCG
ATGCATGCTGATACCGTACAAATTTCTCGCGCCAGGGAAGCCCGACGTGAACTGGAAGAGATTT
TCCAGCGTGAAATGCAACAAGAACGACGCCAGCTGGACGGCTGGGATGAATTTGAGT
SEQ ID NO:8

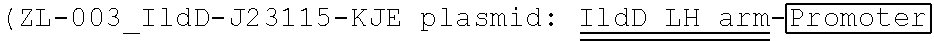

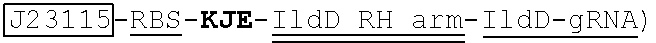

CCACCTGACGTCTAAGAAACCATTATTATCATGACATTAACCTATAAAAATAGGCGTATCACGA
GGCAGAATTTCAGATAAAAAAAATCCTTAGCTTTCGCTAAGGATGATTTCTGGAATTCGCGGCC
GCATTTCCGCAGCCAGCGATTATCGCGCCGCAGCGCAACGCATTCTGCCGCCGTTCCTGTTCCA
CTATATGGATGGGGGGGCATATTCTGAATACACGCTGCGCCGCAACGTGGAAGATTTGTCAGAA
GTGGCGCTGCGCCAGCGTATTCTGAAAAACATGTCTGACTTAAGCCTGGAAACGACGCTGTTTA
ATGAGAAATTGTCGATGCCGGTGGCGCTAGGTCCGGTAGGTTTGTGTGGCATGTATGCGCGACG
CGGCGAAGTTCAGGCTGCCAAAGCAGCAGATGCGCATGGCATTCCGTTTACTCTCTCGACGGTT
TCCGTTTGCCCGATTGAAGAAGTGGCTCCGGCTATCAAACGTCCGATGTGGTTCCAGCTTTATG
TGCTGCGCGATCGCGGCTTTATGCGTAACGCCCTGGAGCGAGCAAAAGCCGCGGGTTGTTCGAC

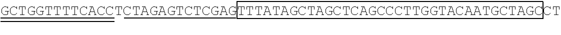

CGAGGAAAGAGGAGAAAGGTCTCAATGGGTAAAATAATTGGTATCGACCTGGGTACTACCAACT
CTTGTGTAGCGATTATGGATGGCACCACTCCTCGCGTGCTGGAGAACGCCGAAGGCGATCGCAC
CACGCCTTCTATCATTGCCTATACCCAGGATGGTGAAACTCTAGTTGGTCAGCCGGCTAAACGT
CAGGCAGTGACGAACCCGCAAAACACTCTGTTTGCGATTAAACGCCTGATTGGTCGCCGCTTCC
AGGACGAAGAAGTACAGCGTGATGTTTCCATCATGCCGTTCAAAATTATTGCTGCTGATAACGG
CGACGCATGGGTCGAAGTTAAAGGCCAGAAAATGGCACCGCCGCAGATTTCTGCTGAAGTGCTG
AAAAAAATGAAGAAAACCGCTGAAGATTACCTGGGTGAACCGGTAACTGAAGCTGTTATCACCG
TACCGGCATACTTTAACGATGCTCAGCGTCAGGCAACCAAAGACGCAGGCCGTATCGCTGGTCT
GGAAGTAAAACGTATCATCAACGAACCGACCGCAGCTGCGCTGGCTTACGGTCTGGACAAAGGC
ACTGGCAACCGTACTATCGCGGTTTATGACCTGGGTGGTGGTACTTTCGATATTTCTATTATCG
AAATCGACGAAGTTGACGGCGAAAAAACCTTCGAAGTTCTGGCAACCAACGGTGATACCCACCT
GGGGGGTGAAGACTTCGACAGCCGTCTGATCAACTATCTGGTTGAAGAATTCAAGAAAGATCAG
GGCATTGACCTGCGCAACGATCCGCTGGCAATGCAGCGCCTGAAAGAAGCGGCAGAAAAAGCGA
AAATCGAACTGTCTTCCGCTCAGCAGACCGACGTTAACCTGCCATACATCACTGCAGACGCGAC
CGGTCCGAAACACATGAACATCAAAGTGACTCGTGCGAAACTGGAAAGCCTGGTTGAAGATCTG
GTAAACCGTTCCATTGAGCCGCTGAAAGTTGCACTGCAGGACGCTGGCCTGTCCGTATCTGATA
TCGACGACGTTATCCTCGTTGGTGGTCAGACTCGTATGCCAATGGTTCAGAAGAAAGTTGCTGA
GTTCTTTGGTAAAGAGCCGCGTAAAGACGTTAACCCGGACGAAGCTGTAGCAATCGGTGCTGCT
GTTCAGGGTGGTGTTCTGACTGGTGACGTAAAAGACGTACTGCTGCTGGACGTTACCCCGCTGT
CTCTGGGTATCGAAACCATGGGCGGTGTGATGACGACGCTGATCGCGAAAAACACCACTATCCC
GACCAAGCACAGCCAGGTGTTCTCTACCGCTGAAGACAACCAGTCTGCGGTAACCATCCATGTG
CTGCAGGGTGAACGTAAACGTGCGGCTGATAACAAATCTCTGGGTCAGTTCAACCTAGATGGTA
TCAACCCGGCACCGCGCGGCATGCCGCAGATCGAAGTTACCTTCGATATCGATGCTGACGGTAT
CCTGCACGTTTCCGCGAAAGATAAAAACAGCGGTAAAGAGCAGAAGATCACCATCAAGGCTTCT
TCTGGTCTGAACGAAGATGAAATCCAGAAAATGGTACGCGACGCAGAAGCTAACGCCGAAGCTG
ACCGTAAGTTTGAAGAGCTGGTACAGACTCGCAACCAGGGCGACCATCTGCTGCACAGCACCCG
TAAGCAGGTTGAAGAAGCAGGCGACAAACTGCCGGCTGACGACAAAACTGCTATCGAGTCTGCG
CTGACTGCACTGGAAACTGCTCTGAAAGGTGAAGACAAAGCCGCTATCGAAGCGAAAATGCAGG
AACTGGCACAGGTTTCCCAGAAACTGATGGAAATCGCCCAGCAGCAACATGCCCAGCAGCAGAC
TGCCGGTGCTGATGCTTCTGCAAACAACGCGAAAGATGACGATGTTGTCGACGCTGAATTTGAA
GAAGTCAAAGACAAAAAATAATCGCCCTATAAACGGGTAATTATACTGACACGGGCGAAGGGGA
ATTTCCTCCCCGCCCGTGCATTCATCTAGGGGCAATTTAAAAAAGATGGCTAAGCAAGATTATT
ACGAGATTTTAGGCGTTTCCAAAACAGCGGAAGAGCGTGAAATCAGAAAGGCCTACAAACGCCT
GGCCATGAAATACCACCCGGACCGTAACCAGGGTGACAAAGAGGCCGAGGCGAAATTTAAAGAG
ATCAAGGAAGCTTATGAAGTTCTGACCGACTCGCAAAAACGTGCGGCATACGATCAGTATGGTC
ATGCTGCGTTTGAGCAAGGTGGCATGGGCGGCGGCGGTTCTGGCGGCGGCGCAGACTTCAGCGA
TATTTTTGGTGACGTTTTCGGCGATATTTTTGGCGGCGGACGTGGTCGTCAACGTGCGGCGCGC
GGTGCTGATTTACGCTATAACATGGAGCTCACCCTCGAAGAAGCTGTACGTGGCGTGACCAAAG
AGATCCGCATTCCGACTCTGGAAGAGTGTGACGTTTGCCACGGTAGCGGTGCAAAACCAGGTAC
ACAGCCGCAGACTTGTCCGACCTGTCATGGTTCTGGTCAGGTGCAGATGCGCCAGGGATTCTTC
GCTGTACAGCAGACCTGTCCACACTGTCAGGGCCGCGGTACGCTGATCAAAGATCCGTGCAACA
AATGTCATGGTCATGGTCGTGTTGAGCGCAGCAAAACGCTGTCCGTTAAAATCCCGGCAGGGGT
GGACACTGGAGACCGCATCCGTCTTGCGGGCGAAGGTGAAGCGGGCGAGCATGGCGCACCGGCA
GGCGATCTGTACGTTCAGGTTCAGGTTAAACAGCACCCGATTTTCGAGCGTGAAGGCAACAACC
TGTATTGCGAAGTCCCGATCAACTTCGCTATGGCGGCGCTGGGTGGCGAAATCGAAGTACCGAC
CCTTGATGGTCGCGTCAAACTGAAAGTGCCTGGCGAAACCCAGACCGGTAAGCTATTCCGTATG
CGCGGTAAAGGCGTCAAGTCTGTCCGCGGTGGCGCGCAGGGTGATTTGCTGTGCCGCGTTGTCG
TCGAAACACCGGTAGGCCTGAACGAAAGGCAGAAACAGCTGCTGCAAGAGCTGCAAGAAAGCTT
CGGTGGCCCAACCGGCGAGCACAACAGCCCGCGCTCAAAGAGCTTCTTTGATGGTGTGAAGAAG
TTTTTTGACGACCTGACCCGCTAACCTCGCGGAGAAATTCATGAGTAGTAAAGAACAGAAAACG
CCTGAGGGGCAAGCCCCGGAAGAAATTATCATGGATCAGCACGAAGAGATTGAGGCAGTTGAGC
CAGAAGCTTCTGCTGAGCAGGTGGATCCGCGCGATGAAAAAGTTGCGAATCTCGAAGCTCAGCT
GGCTGAAGCCCAGACCCGTGAACGTGACGGCATTTTGCGTGTAAAAGCCGAAATGGAAAACCTG
CGTCGTCGTACTGAACTGGATATTGAAAAAGCCCACAAATTCGCGCTGGAGAAATTCATCAACG
AATTGCTGCCGGTGATTGATAGCCTGGATCGTGCGCTGGAAGTGGCTGATAAAGCTAACCCGGA
TATGTCTGCGATGGTTGAAGGCATTGAGCTGACGCTGAAGTCGATGCTGGATGTTGTGCGTAAG
TTTGGCGTTGAAGTGATCGCCGAAACTAACGTCCCACTGGACCCGAATGTGCATCAGGCCATCG
CAATGGTGGAATCTGATGACGTTGCGCCAGGTAACGTACTGGGCATTATGCAGAAGGGTTATAC
GCTGAATGGTCGTACGATTCGTGCGGCGATGGTTACTGTAGCGAAAGCAAAAGCTTAATAACGC
TGATAGTGCTAGTGTAGATCGCTACTAGAGCCAGGCATCAAATAAAACGAAAGGCTCAGTCGAA
AGACTGGGCCTTTCGTTTTATCTGTTGTTTGTCGGTGAACGCTCTCTACTAGAGTCACACTGGC
TCACCTTCGGGTGGGCCTTTCTGCGTTTATATACTAGAAGCGGCCGCTGCAGATCCGCGATTTC
TGGGATGGCCCGATGGTGATCAAAGGGATCCTCGATCCGGAAGATGCGCGCGATGCAGTACGTT
TTGGTGCTGATGGAATTGTGGTTTCTAACCACGGTGGCCGCCAGTTAGATGGCGTACTCTCTTC
TGCTCGTGCACTGCCTGCTATTGCGGATGCGGTGAAAGGTGATATCGCCATTCTGGCGGATAGC
GGAATACGTAACGGGCTTGATGTCGTGCGTATGATTGCGCTCGGTGCCGACACCGTACTGCTGG
GTCGTGCTTTCCTGTATGCACTGGCAACAGCGGGCCAGGCGGGTGTAGCTAATCTGCTAAATCT
GATCGAAAAAGAGATGAAAGTGGCGATGACGCTGACTGGCGCGAAATCGATTAGCGAAATTACG
CAAGATTCGCTGGTGCAGGGGCTGGGTAAAGAGTTGCCTGCGGCATGCAGGAATTCAAAAAAAG
CACCGACTCGGTGCCACTTTTTCAAGTTGATAACGGACTAGCCTTATTTTAACTTGCTATTTCT
AGCTCTAAAACAGAATGCGCATCACGATAACACTAGTATTATACCTAGGACTGAGCTAGCTGTC
AAGGATCCAGCATATGCGGTGTGAAATACCGCACAGATGCGTAAGGAGAAAATACCGCATCAGG
CGCTCTTCCGCTTCCTCGCTCACTGACTCGCTGCGCTCGGTCGTTCGGCTGCGGCGAGCGGTAT
CAGCTCACTCAAAGGCGGTAATACGGTTATCCACAGAATCAGGGGATAACGCAGGAAAGAACAT
GTGAGCAAAAGGCCAGCAAAAGGCCAGGAACCGTAAAAAGGCCGCGTTGCTGGCGTTTTTCCAT
AGGCTCCGCCCCCCTGACGAGCATCACAAAAATCGACGCTCAAGTCAGAGGTGGCGAAACCCGA
CAGGACTATAAAGATACCAGGCGTTTCCCCCTGGAAGCTCCCTCGTGCGCTCTCCTGTTCCGAC
CCTGCCGCTTACCGGATACCTGTCCGCCTTTCTCCCTTCGGGAAGCGTGGCGCTTTCTCATAGC
TCACGCTGTAGGTATCTCAGTTCGGTGTAGGTCGTTCGCTCCAAGCTGGGCTGTGTGCACGAAC
CCCCCGTTCAGCCCGACCGCTGCGCCTTATCCGGTAACTATCGTCTTGAGTCCAACCCGGTAAG
ACACGACTTATCGCCACTGGCAGCAGCCACTGGTAACAGGATTAGCAGAGCGAGGTATGTAGGC
GGTGCTACAGAGTTCTTGAAGTGGTGGCCTAACTACGGCTACACTAGAAGGACAGTATTTGGTA
TCTGCGCTCTGCTGAAGCCAGTTACCTTCGGAAAAAGAGTTGGTAGCTCTTGATCCGGCAAACA
AACCACCGCTGGTAGCGGTGGTTTTTTTGTTTGCAAGCAGCAGATTACGCGCAGAAAAAAAGGA
TCTCAAGAAGATCCTTTGATCTTTTCTACGGGGTCTGACGCTCAGTGGAACGAAAACTCACGTT
AAGGGATTTTGGTCATGAGATTATCAAAAAGGATCTTCACCTAGATCCTTTTAAATTAAAAATG
AAGTTTTAAATCAATCTAAAGTATATATGAGTAAACTTGGTCTGACAGTTACCAATGCTTAATC
AGTGAGGCACCTATCTCAGCGATCTGTCTATTTCGTTCATCCATAGTTGCCTGACTCCCCGTCG
TGTAGATAACTACGATACGGGAGGGCTTACCATCTGGCCCCAGTGCTGCAATGATACCGCGTGA
CCCACGCTCACCGGCTCCAGATTTATCAGCAATAAACCAGCCAGCCGGAAGGGCCGAGCGCAGA
AGTGGTCCTGCAACTTTATCCGCCTCCATCCAGTCTATTAATTGTTGCCGGGAAGCTAGAGTAA
GTAGTTCGCCAGTTAATAGTTTGCGCAACGTTGTTGCCATTGCTACAGGCATCGTGGTGTCACG
CTCGTCGTTTGGTATGGCTTCATTCAGCTCCGGTTCCCAACGATCAAGGCGAGTTACATGATCC
CCCATGTTGTGCAAAAAAGCGGTTAGCTCCTTCGGTCCTCCGATCGTTGTCAGAAGTAAGTTGG
CCGCAGTGTTATCACTCATGGTTATGGCAGCACTGCATAATTCTCTTACTGTCATGCCATCCGT
AAGATGCTTTTCTGTGACTGGTGAGTACTCAACCAAGTCATTCTGAGAATAGTGTATGCGGCGA
CCGAGTTGCTCTTGCCCGGCGTCAATACGGGATAATACCGCGCCACATAGCAGAACTTTAAAAG
TGCTCATCATTGGAAAACGTTCTTCGGGGCGAAAACTCTCAAGGATCTTACCGCTGTTGAGATC
CAGTTCGATGTAACCCACTCGTGCACCCAACTGATCTTCAGCATCTTTTACTTTCACCAGCGTT
TCTGGGTGAGCAAAAACAGGAAGGCAAAATGCCGCAAAAAAGGGAATAAGGGCGACACGGAAAT
GTTGAATACTCATACTCTTCCTTTTTCAATATTATTGAAGCATTTATCAGGGTTATTGTCTCAT
GAGCGGATACATATTTGAATGTATTTAGAAAAATAAACAAATAGGGGTTCCGCGCACATTTCCC
CGAAAAGTG
SEQ ID NO:9

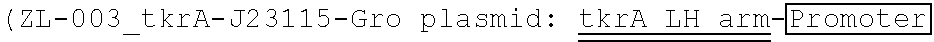

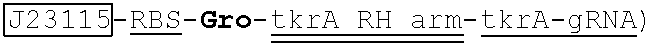

CCACCTGACGTCTAAGAAACCATTATTATCATGACATTAACCTATAAAAATAGGCGTATCACGA
GGCAGAATTTCAGATAAAAAAAATCCTTAGCTTTCGCTAAGGATGATTTCTGGAATTCGCGGCC
GCATCTGCTGATGCACACACCAACCGTATTAACAGAAACCGTCGCCGATACGCTGATGGCGCTG
GTGTTGTCTACCGCTCGTCGGGTTGTGGAGGTAGCAGAACGGGTAAAAGCAGGCGAATGGACCG
CGAGCATAGGCCCGGACTGGTACGGCACTGACGTTCACCATAAAACACTGGGCATTGTCGGGAT
GGGACGGATCGGCATGGCGCTGGCACAACGTGCGCACTTTGGCTTCAACATGCCCATCCTCTAT
AACGCGCGTCGCCACCATAAAGAAGCAGAAGAACGCTTCAACGCCCGCTACTGCAATTTGGATA
CTCTGTTACAAGAGTCAGATTTCGTTTGCCTGATCCTGCCGTTAACGGATGAGACGCATCATCT
GTTTGGCGCAGAACAATTCGCCAAAATGAAATCCTCCGCCATTTTCATTAATGCCGGACGTGGC
CCGGTGGTTGATGAAAATGCACTGATTGCTGCATTGCAGAAAGGGGAAATTCACGCCGTCTAGA

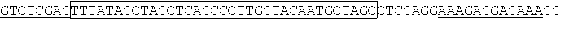

TCTCAATGAATATTCGTCCATTGCATGATCGCGTGATCGTCAAGCGTAAAGAAGTTGAAACTAA
ATCTGCTGGCGGCATCGTTCTGACCGGCTCTGCAGCGGCTAAATCCACCCGCGGCGAAGTGCTG
GCTGTCGGCAATGGCCGTATCCTTGAAAATGGCGAAGTGAAGCCGCTGGATGTGAAAGTTGGCG
ACATCGTTATTTTCAACGATGGCTACGGTGTGAAATCTGAGAAGATCGACAATGAAGAAGTGTT
GATCATGTCCGAAAGCGACATTCTGGCAATTGTTGAAGCGTAATCCGCGCACGACACTGAACAT
ACGAATTTAAGGAATAAAGATAATGGCAGCTAAAGACGTAAAATTCGGTAACGACGCTCGTGTG
AAAATGCTGCGCGGCGTAAACGTACTGGCAGATGCAGTGAAAGTTACCCTCGGTCCAAAAGGCC
GTAACGTAGTTCTGGATAAATCTTTCGGTGCACCGACCATCACCAAAGATGGTGTTTCCGTTGC
TCGTGAAATCGAACTGGAAGACAAGTTCGAAAATATGGGTGCGCAGATGGTGAAAGAAGTTGCC
TCTAAAGCAAACGACGCTGCAGGCGACGGTACCACCACTGCAACCGTACTGGCTCAGGCTATCA
TCACTGAAGGTCTGAAAGCTGTTGCTGCGGGCATGAACCCGATGGACCTGAAACGTGGTATCGA
CAAAGCGGTTACCGCTGCAGTTGAAGAACTGAAAGCGCTGTCCGTACCATGCTCTGACTCTAAA
GCGATTGCTCAGGTTGGTACCATCTCCGCTAACTCCGACGAAACCGTAGGTAAACTGATCGCTG
AAGCGATGGACAAAGTCGGTAAAGAAGGCGTTATCACCGTTGAAGACGGTACCGGTCTGCAGGA
CGAACTGGACGTGGTTGAAGGTATGCAGTTCGACCGTGGCTACCTGTCTCCTTACTTCATCAAC
AAGCCGGAAACTGGCGCAGTAGAACTGGAAAGCCCGTTCATCCTGCTGGCTGACAAGAAAATCT
CCAACATCCGCGAAATGCTGCCGGTTCTGGAAGCTGTTGCCAAAGCAGGCAAACCGCTGCTGAT
CATCGCTGAAGATGTAGAAGGCGAAGCGCTGGCAACTCTGGTTGTTAACACCATGCGTGGCATC
GTGAAAGTCGCTGCGGTTAAAGCACCGGGCTTCGGCGATCGTCGTAAAGCTATGCTGCAGGATA
TCGCAACCCTGACTGGCGGTACCGTGATCTCTGAAGAGATCGGTATGGAGCTGGAAAAAGCAAC
CCTGGAAGACCTGGGTCAGGCTAAACGTGTTGTGATCAACAAAGACACCACCACTATCATCGAT
GGCGTGGGTGAAGAAGCTGCAATCCAGGGCCGTGTTGCTCAGATCCGTCAGCAGATTGAAGAAG
CAACTTCTGACTACGACCGTGAAAAACTGCAGGAACGCGTAGCGAAACTGGCAGGCGGCGTTGC
AGTTATCAAAGTGGGTGCTGCTACCGAAGTTGAAATGAAAGAGAAAAAAGCACGCGTTGAAGAT
GCCCTGCACGCGACCCGTGCTGCGGTAGAAGAAGGCGTGGTTGCTGGTGGTGGTGTTGCGCTGA
TCCGCGTAGCGTCTAAACTGGCTGACCTGCGTGGTCAGAACGAAGACCAGAACGTGGGTATCAA
AGTTGCACTGCGTGCAATGGAAGCTCCGCTGCGTCAGATCGTATTGAACTGCGGCGAAGAACCG
TCTGTTGTTGCTAACACCGTTAAAGGCGGCGACGGCAACTACGGTTACAACGCAGCAACCGAAG
AATACGGCAACATGATCGACATGGGTATCCTGGATCCAACCAAAGTAACTCGTTCTGCTCTGCA
GTACGCAGCTTCTGTGGCTGGCCTGATGATCACCACCGAATGCATGGTTACCGACCTGCCGAAA
AACGATGCAGCTGACTTAGGCGCTGCTGGCGGTATGGGCGGCATGGGTGGCATGGGCGGCATGA
TGTAATTGCCCTGCACCTCGCAGAAATAAACAAACCCCCCTGTGATTTTTTGAGGTAACAAGAT
GCAAGTTTCAGTTGAAACCACTCAAGGCCTTGGCCGCCGTGTAACGATTACTATCGCTGCTGAC
AGCATCGAGACCGCTGTTAAAAGCGAGCTGGTCAACGTTGCGAAAAAAGTACGTATTGACGGCT
TCCGCAAAGGCAAAGTGCCAATGAATATCGTTGCTCAGCGTTATGGCGCGTCTGTACGCCAGGA
CGTTCTGGGTGACCTGATGAGCCGTAACTTCATTGACGCCATCATTAAAGAAAAAATCAATCCG
GCTGGCGCACCGACTTATGTTCCGGGCGAATACAAGCTGGGTGAAGACTTCACTTACTCTGTAG
AGTTTGAAGTTTATCCGGAAGTTGAACTGCAGGGTCTGGAAGCGATCGAAGTTGAAAAACCGAT
CGTTGAAGTGACCGACGCTGACGTTGACGGCATGCTGGATACTCTGCGTAAACAGCAGGCGACC
TGGAAAGAAAAAGACGGCGCTGTTGAAGCAGAAGACCGCGTAACCATCGACTTCACCGGTTCTG
TAGACGGCGAAGAGTTCGAAGGCGGTAAAGCGTCTGATTTCGTACTGGCGATGGGCCAGGGTCG
TATGATCCCGGGCTTTGAAGACGGTATCAAAGGCCACAAAGCTGGCGAAGAGTTCACCATCGAC
GTGACCTTCCCGGAAGAATACCACGCAGAAAACCTGAAAGGTAAAGCAGCGAAATTCGCTATCA
ACCTGAAGAAAGTTGAAGAGCGTGAACTGCCGGAACTGACTGCAGAATTCATCAAACGTTTCGG
CGTTGAAGATGGTTCCGTAGAAGGTCTGCGCGCTGAAGTGCGTAAAAACATGGAGCGCGAGCTG
AAGAGCGCCATCCGTAACCGCGTTAAGTCTCAGGCGATCGAAGGTCTGGTAAAAGCTAACGACA
TCGACGTACCGGCTGCGCTGATCGACAGCGAAATCGACGTTCTGCGTCGCCAGGCTGCACAGCG
TTTCGGTGGCAACGAAAAACAAGCTCTGGAACTGCCGCGCGAACTGTTCGAAGAACAGGCTAAA
CGCCGCGTAGTTGTTGGCCTGCTGCTGGGCGAAGTTATCCGCACCAACGAGCTGAAAGCTGACG
AAGAGCGCGTGAAAGGCCTGATCGAAGAGATGGCTTCTGCGTACGAAGATCCGAAAGAAGTTAT
CGAGTTCTACAGCAAAAACAAAGAACTGATGGACAACATGCGCAATGTTGCTCTGGAAGAACAG
GCTGTTGAAGCTGTACTGGCGAAAGCGAAAGTGACTGAAAAAGAAACCACTTTCAACGAGCTGA
TGAACCAGCAGGCGTAATAACGCTGATAGTGCTAGTGTAGATCGCTACTAGAGCCAGGCATCAA
ATAAAACGAAAGGCTCAGTCGAAAGACTGGGCCTTTCGTTTTATCTGTTGTTTGTCGGTGAACG
CTCTCTACTAGAGTCACACTGGCTCACCTTCGGGTGGGCCTTTCTGCGTTTATATACTAGAAGC
GGCCGCTGCAGTCGAACAAGAGCCACTGTCCGTAGATTCGCCGTTGCTCTCAATGGCCAACGTC
GTCGCAGTACCGCATATTGGATCTGCCACTCATGAGACGCGTTATGGCATGGCCGCCTGTGCCG
TGGATAATTTGATTGATGCGTTACAAGGAAAGGTTGAGAAGAACTGTGTGAATCCGCACGTCGC
GGACTAAGCCGCGACTGCGTGGAGTAAAGCCCGATAATCGCTCGGGCTTTTACTCTTTATTGGG
TTGCAGTAACTGCTGTAGTCCAGGCCTGATTAAACGCCTGATGTTGTGCCGGTAATGGCGCAAT
CAGTTTGTTATATTCACTTGCCTGCTGTGAAGTCGGGAACTTGCAGGAATTCAAAAAAAGCACC
GACTCGGTGCCACTTTTTCAAGTTGATAACGGACTAGCCTTATTTTAACTTGCTATTTCTAGCT
CTAAAACCCGGGCTGGATGTCTTCGAAACTAGTATTATACCTAGGACTGAGCTAGCTGTCAAGG
ATCCAGCATATGCGGTGTGAAATACCGCACAGATGCGTAAGGAGAAAATACCGCATCAGGCGCT
CTTCCGCTTCCTCGCTCACTGACTCGCTGCGCTCGGTCGTTCGGCTGCGGCGAGCGGTATCAGC
TCACTCAAAGGCGGTAATACGGTTATCCACAGAATCAGGGGATAACGCAGGAAAGAACATGTGA
GCAAAAGGCCAGCAAAAGGCCAGGAACCGTAAAAAGGCCGCGTTGCTGGCGTTTTTCCATAGGC
TCCGCCCCCCTGACGAGCATCACAAAAATCGACGCTCAAGTCAGAGGTGGCGAAACCCGACAGG
ACTATAAAGATACCAGGCGTTTCCCCCTGGAAGCTCCCTCGTGCGCTCTCCTGTTCCGACCCTG
CCGCTTACCGGATACCTGTCCGCCTTTCTCCCTTCGGGAAGCGTGGCGCTTTCTCATAGCTCAC
GCTGTAGGTATCTCAGTTCGGTGTAGGTCGTTCGCTCCAAGCTGGGCTGTGTGCACGAACCCCC
CGTTCAGCCCGACCGCTGCGCCTTATCCGGTAACTATCGTCTTGAGTCCAACCCGGTAAGACAC
GACTTATCGCCACTGGCAGCAGCCACTGGTAACAGGATTAGCAGAGCGAGGTATGTAGGCGGTG
CTACAGAGTTCTTGAAGTGGTGGCCTAACTACGGCTACACTAGAAGGACAGTATTTGGTATCTG
CGCTCTGCTGAAGCCAGTTACCTTCGGAAAAAGAGTTGGTAGCTCTTGATCCGGCAAACAAACC
ACCGCTGGTAGCGGTGGTTTTTTTGTTTGCAAGCAGCAGATTACGCGCAGAAAAAAAGGATCTC
AAGAAGATCCTTTGATCTTTTCTACGGGGTCTGACGCTCAGTGGAACGAAAACTCACGTTAAGG
GATTTTGGTCATGAGATTATCAAAAAGGATCTTCACCTAGATCCTTTTAAATTAAAAATGAAGT
TTTAAATCAATCTAAAGTATATATGAGTAAACTTGGTCTGACAGTTACCAATGCTTAATCAGTG
AGGCACCTATCTCAGCGATCTGTCTATTTCGTTCATCCATAGTTGCCTGACTCCCCGTCGTGTA
GATAACTACGATACGGGAGGGCTTACCATCTGGCCCCAGTGCTGCAATGATACCGCGTGACCCA
CGCTCACCGGCTCCAGATTTATCAGCAATAAACCAGCCAGCCGGAAGGGCCGAGCGCAGAAGTG
GTCCTGCAACTTTATCCGCCTCCATCCAGTCTATTAATTGTTGCCGGGAAGCTAGAGTAAGTAG
TTCGCCAGTTAATAGTTTGCGCAACGTTGTTGCCATTGCTACAGGCATCGTGGTGTCACGCTCG
TCGTTTGGTATGGCTTCATTCAGCTCCGGTTCCCAACGATCAAGGCGAGTTACATGATCCCCCA
TGTTGTGCAAAAAAGCGGTTAGCTCCTTCGGTCCTCCGATCGTTGTCAGAAGTAAGTTGGCCGC
AGTGTTATCACTCATGGTTATGGCAGCACTGCATAATTCTCTTACTGTCATGCCATCCGTAAGA
TGCTTTTCTGTGACTGGTGAGTACTCAACCAAGTCATTCTGAGAATAGTGTATGCGGCGACCGA
GTTGCTCTTGCCCGGCGTCAATACGGGATAATACCGCGCCACATAGCAGAACTTTAAAAGTGCT
CATCATTGGAAAACGTTCTTCGGGGCGAAAACTCTCAAGGATCTTACCGCTGTTGAGATCCAGT
TCGATGTAACCCACTCGTGCACCCAACTGATCTTCAGCATCTTTTACTTTCACCAGCGTTTCTG
GGTGAGCAAAAACAGGAAGGCAAAATGCCGCAAAAAAGGGAATAAGGGCGACACGGAAATGTTG
AATACTCATACTCTTCCTTTTTCAATATTATTGAAGCATTTATCAGGGTTATTGTCTCATGAGC
GGATACATATTTGAATGTATTTAGAAAAATAAACAAATAGGGGTTCCGCGCACATTTCCCCGAA
AAGTG

### Example 4. Detection of Acetaldehyde Metabolic Activity of the Engineered Bacterial Strains

### 1. Detection of the Acetaldehyde Tolerance of the Engineered Bacterial Strains

Single colonies were picked from control bacteria, the engineered bacteria 119 and engineered bacteria 101, and were plated on LB agar plates containing 0 mM, 5 mM, 10 mM, 15 mM, 20 mM, and 30 mM acetaldehyde respectively and grown at 37° C. overnight. The results showed that both the control bacteria, the engineered bacteria 119 and engineered bacteria 101 can grow normally even at an acetaldehyde concentration of 15 mM (FIG. 3).

### 2. Detection of the Acetaldehyde Metabolic Activity of the Engineered Bacterial Strains in Vitro

1) Preparation of Engineered Bacteria Reaction Samples

(1) A certain amount of bacteria were centrifuged at room temperature, and were resuspended in 1500 μL 10 mM acetaldehyde (prepared with aseptic water) and reacted at 37° C. for 1 h.

(2) 100 μl supernatant was harvested after centrifugation, and was diluted for 10 times in 900 μL aseptic water for further detection.

2) Preparation of Standard Samples:

(1) 2M acetaldehyde was diluted with aseptic water to reach 10 mM for reaction with engineered bacteria culture medium.

(2) 2M acetaldehyde was diluted with aseptic water to reach 1 mM, 0.5 mM and 0.25 mM for us as HPLC standards.

3) Acetaldehyde Derivation

(1) Preparation of derivation reagent: Added 25 ml of 10% hydrochloric acid (23 ml 35% HCl+77 ml H₂O) into 12 mg of 2,4-dinitrophenylhydrazine (2,4-DNPH, Sangon Biotech (Shanghai) Co., Ltd., analytical reagent) and dissolved by ultrasonication to achieve derivation reagent.

(2) Derivation Reaction: in a 1.5 mL EP tube, added 900 μl ultra-pure water, 300 μl engineered bacterial reaction sample or standard solution, and then added 300 μl 2,4-dinitrophenylhydrazine solution prepared according to the method above. The tube was then incubated at 60° C. for 60 minutes after mixing well. After cooling down on ice, filtration with 0.22 μm water system filtration membrane was applied immediately before HPLC detection.

(3) HPLC detection: 20 μL samples were run on an Athena C18 HPLC column (Anpel Laboratory Technologies (Shanghai) Inc., 4.6×250 mm, 5 μm) for HPLC detection. Samples were run at 1.0 mL·min⁻¹ with H₂O: acetonitrile (40:60) as the mobile phase and 40° C. as the column temperature. Samples were detected at a detection wavelength of 360 nm.

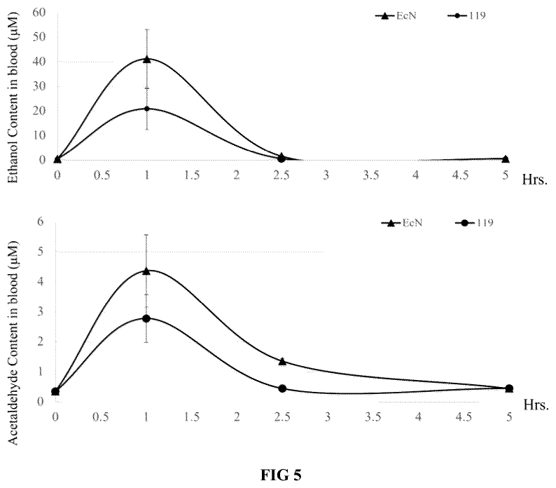

(4) Detection results: FIG. 4 shows that compared with control strain, the remaining acetaldehyde after lh reaction with 6×10⁹ CFU of strain 119 was lower than 1 mM; the remaining acetaldehyde after 1.5 h reaction with 6×10⁹ CFU of strain 101 was lower than 1 mM; the remaining acetaldehyde after 1.5 h reaction with 6×10⁹ CFU of strain 108 was 4.4 mM. This indicates the promoters' strength would affect the activity of aldehyde dehydrogenase. It is generally considered that stronger promoters are more likely to negatively affect the growth of bacterial strains. However, the inventors of the present disclosure surprisingly found that both of the bacterial strain comprising the strongest promoter J23119 and the bacterial strain comprising the relatively weaker promoter J23101 can grow well and can tolerate acetaldehyde at a concentration up to 15 mM (FIG. 3A). The inventors of the present disclosure further surprisingly found that selecting EcN as a chassis bacteria is advantageous over other bacterial strains, e.g., Bacillus subtilis. FIG. 4 shows that 6×10⁹ CFU of strain 119 of the present invention can consume about 90% acetaldehyde after reacting in 10 mM acetaldehyde for 1 h, which is much more efficient in acetaldehyde degradation than engineered Bacillus subtilis (only about 40% acetaldehyde was degraded with the same amount of bacteria and incubated for the same amount of time) as shown in ZBiotics et al., US2019076489A1.

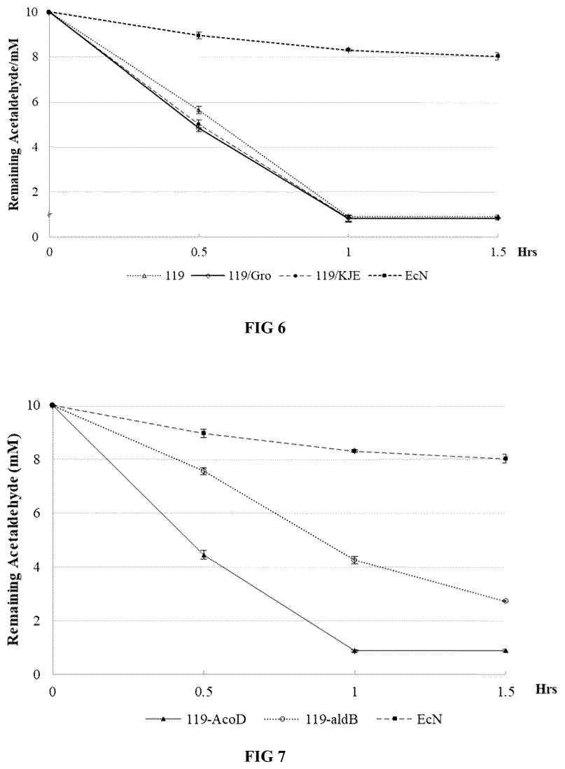

FIG. 6 shows that with a starting concentration of acetaldehyde of 10 mM, compared with control strain, both of the engineered bacteria 119/Gro overexpressing molecular chaperone groES-groEL-tig, and the engineered bacteria 119/KJE overexpressing dnaK-dnaJ-grpE consumed about 9 mM acetaldehyde in 1 h. These data suggested that overexpressing molecular chaperons could promote the catalytic activity of acetaldehyde dehydrogenases to some extent, but the effect was not significant.

### Example 5. Optimization of the Engineered Bacterial Strains

### 1. Acetaldehyde Dehydrogenase

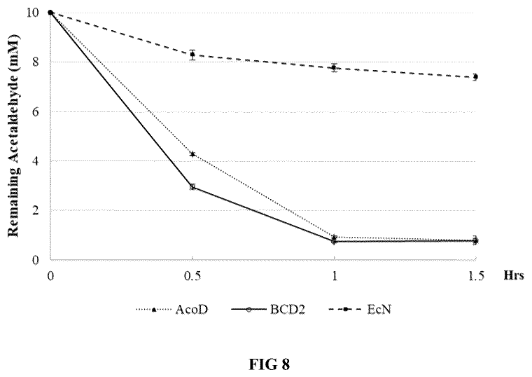

Considering that strain Nissle 1917 itself expresses acetaldehyde dehydrogenase (AldB) gene as well, as shown in the previous results, the efficiency of acetaldehyde metabolism by strain Nissle 1917 alone was very low, we speculated that increasing the expression level of AldB might also improve the activity of acetaldehyde metabolism by strains. Thus, in this example, a single-copy J23119-AldB expression cassette (sequence of which was set forth in SEQ ID NO: 112) was inserted into the kef13 site of the genome of Nissle 1917 to construct an engineered strain overexpressing AldB. The results were shown in FIG. 7: compared with wildtype Nissle 1917 strain, the efficiency of acetaldehyde metabolism in engineered strain overexpressing AldB was significantly improved. After reaction for 1 h in 10 mM acetaldehyde, about 5 mM acetaldehyde could be degraded, suggesting that the increasing the expression of E. coli endogenous AldB gene could indeed improve the activity of acetaldehyde metabolism. However, the engineered strain 119 inserted with J23119-AcoD expression cassette could metabolize 9 mM acetaldehyde within the same time, which was much higher than that of engineered strain overexpressing AldB. Thus, this example surprisingly validated that the AcoD derived from Cupriavidus necator was more suitable for the construction of engineered strains for acetaldehyde metabolism than the endogenous AldB from the strain Nissle 1917.

The sequence comprising the J23119-AldB expression cassette is as follows, wherein the part in uppercase and roman type is the upstream and downstream homologous fragments of kefB; the italic part is the expression cassette of AldB gene, including the promoter, AldB gene and T7 terminator; the lowercase italic sequence is the J23119 promoter and RBS site; and the uppercase italic underlined sequence is the open reading frame of AldB gene.

(SEQ ID NO: 114)
TTGTTTATGGATGCGCTGGGGTTGTCGATGGCGCTCGGTACGTTTATTGC
GGGTGTGCTACTGGCGGAAAGTGAATATCGCCATGAACTGGAAACGGCTA
TCGATCCCTTCAAAGGCTTGCTGCTCGGTTTGTTCTTTATCTCTGTCGGC
ATGTCGCTCAACCTCGGGGTGCTTTATACCCATCTGTTGTGGGTAGTGAT
AAGTGTGGTTGTGCTGGTGGCGGTGAAAATTCTCGTGCTGTATCTGCTGG
CGCGATTGTATGGCGTGCGCAGTTCTGAGCGGATGCAGTTTGCTGGCGTG
TTGAGTCAGGGCGGTGAGTTTGCCTTTGTCCTCTTTTCTACCGCTTCTTC
ACAACGCTTATTCCAGGGCGACCAGATTctagagtctcgagttgacagct
agctcagtcctaggtataatgctagcctcgaggaaagaggagaaagaagc
ttATGACCAATAATCCCCCTTCAGCACAGATTAAGCCCGGCGAGTATGGT
TTCCCCCTCAAGTTAAAAGCCCGCTATGACAACTTTATTGGCGGCGAATG
GGTAGCCCCTGCCGACGGCGAGTATTACCAGAATCTGACGCCGGTGACCG
GGCAGCTGCTGTGCGAAGTGGCGTCTTCGGGCAAACGAGACATCGATCTG
GCGCTGGATGCTGCGCACAAAGTGAAAGATAAATGGGCGCACACCTCGGT
GCAGGATCGTGCGGCGATTCTGTTTAAGATTGCCGATCGAATGGAACAAA
ACCTCGAGCTGTTAGCGACAGCTGAAACCTGGGATAACGGCAAACCCATT
CGCGAAACCAGTGCTGCGGATGTACCGCTGGCGATTGACCATTTCCGCTA
TTTCGCCTCGTGTATTCGGGCGCAGGAAGGTGGGATCAGTGAAGTTGATA
GCGAAACCGTGGCCTATCATTTCCATGAACCGTTAGGCGTGGTGGGGCAG
ATTATCCCGTGGAACTTCCCGCTGCTGATGGCGAGCTGGAAAATGGCTCC
CGCGCTGGCGGCGGGCAACTGTGTGGTGCTGAAACCCGCACGTCTTACCC
CGCTTTCTGTACTGCTGCTAATGGAAATTGTCGGTGATTTACTGCCGCCG
GGCGTGGTGAACGTGGTCAATGGCGCAGGTGGGGTAATTGGCGAATATCT
GGCGACCTCGAAACGCATCGCCAAAGTGGCGTTTACCGGCTCAACGGAAG
TGGGCCAACAAATTATGCAATACGCAACGCAAAACATTATTCCGGTGACG
CTGGAGTTGGGCGGTAAGTCGCCAAATATCTTCTTTGCTGATGTGATGGA
TGAAGAAGATGCCTTTTTCGATAAAGCGCTGGAAGGCTTTGCACTGTTTG
CCTTTAACCAGGGCGAAGTTTGCACCTGTCCGAGTCGTGCTTTAGTGCAG
GAATCTATCTACGAACGCTTTATGGAACGCGCCATCCGCCGTGTCGAAAG
CATTCGTAGCGGTAACCCGCTCGACAGCGTGACGCAAATGGGCGCGCAGG
TTTCTCACGGGCAACTGGAAACCATCCTCAACTACATTGATATCGGTAAA
AAAGAGGGCGCTGACGTGCTCACAGGCGGGCGGCGCAAGCTGCTGGAAGG
TGAACTGAAAGACGGCTACTACCTCGAACCGACGATTCTGTTTGGTCAGA
ACAATATGCGGGTGTTCCAGGAGGAGATTTTTGGCCCGGTGCTGGCGGTG
ACCACCTTCAAAACGATGGAAGAAGCGCTGGAGCTGGCGAACGATACGCA
ATATGGCCTGGGCGCGGGCGTCTGGAGCCGCAACGGTAATCTGGCCTATA
AGATGGGGCGCGGCATACAGGCTGGGCGCGTGTGGACCAACTGTTATCAC
GCTTACCCGGCACATGCGGCGTTTGGTGGCTACAAACAATCAGGTATCGG
TCGCGAAACCCACAAGATGATGCTGGAGCATTACCAGCAAACCAAGTGCC
TGCTGGTGAGCTACTCGGATAAACCGTTGGGGCTGTTCTGAGAGCTCGAT
AGTGCTAGTGTAGATCGCTACTAGAGCCAGGCATCAAATAAAACGAAAGG
CTCAGTCGAAAGACTGGGCCTTTCGTTTTATCTGTTGTTTGTCGGTGAAC
GCTCTCTACTAGAGTCACACTGGCTCACCTTCGGGTGGGCCTTTCTGCGT
TTATATACTAGAAGCGGCCGCTGCAGTTGCATATTCTTGCGCGAGCGCGC
GGACGTGTGGAAGCGCATGAGTTATTACAGGCAGGGGTGACGCAGTTTTC
CCGTGAAACATTCTCCAGTGCGTTAGAGCTGGGGCGCAAGACGCTGGTCA
CGCTTGGCATGCATCCGCATCAGGCGCAGCGCGCGCAACTGCATTTTCGC
CGCCTGGATATGCGAATGCTGCGGGAGTTAATCCCGATGCATGCTGATAC
CGTACAAATTTCTCGCGCCAGGGAAGCCCGACGTGAACTGGAAGAGATTT
TCCAGCGTGAAATGCAACAAGAACGACGCCAGCTGGACGGCTGGGATGAA
TTTGAG

### 2. Cistron

This example further investigated whether the addition of BCD2 cistron (sequence of which is set forth in SEQ ID NO: 62) upstream the open reading framework (ORF) of AcoD could further improve the activity of acetaldehyde metabolism by engineered strains. A single-copy J23119-BCD2-AcoD expression cassette (sequence of which is set forth in SEQ ID NO: 113) was inserted into the kefB site of the genome of strain Nissle 1917 to construct an engineered strain bearing BCD2 cistron upstream the AcoD open reading frame. The result was shown in FIG. 8: compared with the strain AcoD in absence of BCD2 cistron, the engineered strain having BCD2 only took 45 minutes to metabolize 90% acetaldehyde, while the strain AcoD took lh. This indicated that the addition of a cistron could further improve the acetaldehyde metabolic activity of engineered bacteria.

The sequence comprising the J23119-BCD2-AcoD expression cassette is as follows, wherein the part in uppercase and roman type is the upstream and downstream homologous fragments of kefB; the italic part is the expression cassette of AcoD gene, including the promoter, BCD2 cistron, AcoD gene and T7 terminator; the lowercase italic sequence is the J23119 promoter and RBS site; the uppercase italic underlined sequence is the open reading frame of AcoD gene, and the uppercase italic double-underlined sequence is BCD2 cistron.

(SEQ ID NO: 115)
TTGTTTATGGATGCGCTGGGGTTGTCGATGGCGCTCGGTACGTTTATTGC
GGGTGTGCTACTGGCGGAAAGTGAATATCGCCATGAACTGGAAACGGCTA
TCGATCCCTTCAAAGGCTTGCTGCTCGGTTTGTTCTTTATCTCTGTCGGC
ATGTCGCTCAACCTCGGGGTGCTTTATACCCATCTGTTGTGGGTAGTGAT
AAGTGTGGTTGTGCTGGTGGCGGTGAAAATTCTCGTGCTGTATCTGCTGG
CGCGATTGTATGGCGTGCGCAGTTCTGAGCGGATGCAGTTTGCTGGCGTG
TTGAGTCAGGGCGGTGAGTTTGCCTTTGTCCTCTTTTCTACCGCTTCTTC
ACAACGCTTATTCCAGGGCGACCAGATTctagagtctcgagttgacagct
agctcagtcctaggtataatgctagcctcgaggaaagaggagaaagAAGC
TTATGAAAGCAATTTTCGTACTGAAACATCTTAATCATGCTAAGGAGGTT
TTCTAATGAATATGGCAGAAATTGCCCAGCTGGGTGTGAGTAATCCGTAT
AAACAGCAGTATGAAAATTATATTGGTGGTGCATGGGTTCCGCCAGCTGG
CGGTGAATATTTTGAATCAACCACCCCGATTACCGGCAAACCGTTTACCC
GTGTTCCGCGTAGCGGTCAGCAGGATGTGGATGCCGCACTGGATGCAGCA
CATGCAGCCAAAGCCGCATGGGCACGTACCTCTACCACCGAACGTGCCAA
TATTCTGAATCGCATTGCCGATCGCATTGAAGCCAATCTGAAACTGCTGG
CAGTTGCCGAATCTATTGATAATGGTAAACCGGTTCGTGAAACCACCGCC
GCCGATCTGCCGTTAGCAGTGGATCATTTTCGCTATTTTGCAGGTTGTAT
TCGCGCCCAGGAAGGCGGCATTAGCGAAATTGATGCAGATACCATTGCAT
ATCATTTTCATGAACCGTTAGGCGTTGTGGGCCAGATTATTCCGTGGAAT
TTTCCGCTGTTAATGGCAACCTGGAAACTGGCCCCGGCCTTAGCAGCAGG
TAATTGTGTTGTGCTGAAACCCGCCGAACAGACCCCGGCCTCAATTCTGG
TGTTAATGGAAGTGATTGGCGATTTACTGCCGCCGGGCGTTGTTAATGTG
ATTAATGGCTTTGGCTTAGAAGCAGGTAAACCGCTGGCAAGCTCTCCGCG
CATTTCTAAAGTTGCCTTTACCGGCGAAACCACCACCGGTCGTCTGATTA
TGCAGTATGCAAGTCAGAATCTGATTCCGGTGACCTTAGAACTGGGTGGT
AAAAGTCCGAATATTTTTTTTGAAGATGTGCTGGCCGCCGATGATGCCTT
TTTTGATAAAGCCCTGGAAGGCTTTGCCATGTTTGCACTGAATCAGGGCG
AAGTTTGTACCTGTCCGTCACGCGCACTGATTCAGGAATCAATTTATGAT
CGCTTTATGGAACGCGCCTTAAAACGGGTTGCAGCAATTCGTCAGGGCCA
TCCGTTAGATACCGGTACCATGATTGGCGCACAGGCCTCTGCCGAACAGT
TAGAAAAAATTCTGAGCTATATTGATCTGGGTCGCAAAGAAGGCGCCCAG
TGTCTGACCGGCGGTGAACGTAATGTGCTGGATGGCGATTTAGCCGGTGG
CTATTATGTTAAACCGACCGTGTTTGCAGGTCATAATAAAATGCGCATTT
TTCAGGAAGAAATTTTTGGTCCGGTTGTGAGCGTGACCACCTTTAAAGAT
GAAGAAGAAGCCTTAGCCATTGCCAATGATACCCTGTATGGTTTAGGTGC
AGGCGTGTGGACCCGCGATGGTGCACGCGCCTTTCGTATGGGTCGTGGTA
TTCAGGCAGGTCGCGTTTGGACCAATTGTTATCATGCCTATCCGGCACAT
GCAGCCTTTGGCGGCTATAAACAGAGCGGTATTGGTCGCGAAAATCATCG
TATGATGTTAGATCATTATCAGCAGACCAAAAATCTGTTAGTGTCTTATA
GTCCGAATGCCCTGGGCTTTTTTTAAGAGCTCGATAGTGCTAGTGTAGAT
CGCTACTAGAGCCAGGCATCAAATAAAACGAAAGGCTCAGTCGAAAGACT
GGGCCTTTCGTTTTATCTGTTGTTTGTCGGTGAACGCTCTCTACTAGAGT
CACACTGGCTCACCTTCGGGTGGGCCTTTCTGCGTTTATATACTAGAAGC
GGCCGCTGCAGTTGCATATTCTTGCGCGAGCGCGCGGACGTGTGGAAGCG
CATGAGTTATTACAGGCAGGGGTGACGCAGTTTTCCCGTGAAACATTCTC
CAGTGCGTTAGAGCTGGGGCGCAAGACGCTGGTCACGCTTGGCATGCATC
CGCATCAGGCGCAGCGCGCGCAACTGCATTTTCGCCGCCTGGATATGCGA
ATGCTGCGGGAGTTAATCCCGATGCATGCTGATACCGTACAAATTTCTCG
CGCCAGGGAAGCCCGACGTGAACTGGAAGAGATTTTCCAGCGTGAAATGC
AACAAGAACGACGCCAGCTGGACGGCTGGGATGAATTTGAGT

### 3. Detection of the Acetaldehyde Metabolic Activity of the Engineered Bacterial Strains In Vivo

The animal experiment was entrusted to PharmaLegacy Laboratories (Shanghai) Co., Ltd. Male SD rats aged 8-9 weeks were randomly divided into two groups with 6 rats in each group. Each group was subjected to intragastric administration of 500 μL (5×10¹¹ CFU) control bacterial or engineered bacteria 119. After 3 hours, each rat was orally administered with ethanol at a dose of 2 g/kg body weight (using prepared 60% alcohol(V/10)). At 0, 1, 2.5 and 5 hours after oral administration of ethanol, took blood from jugular vein and collected serum therefrom.

1) Preparation of ethanol/acetaldehyde standard: serum samples were harvested from non-experimental SD rats at the same age, into which a certain concentration of ethanol and acetaldehyde was added (the three standards contained 40 μm ethanol and 4 μm acetaldehyde, 20 μm ethanol and 2 μm acetaldehyde, and 10 μm ethanol and 1 μm acetaldehyde, respectively).

2) Detection of the Contents of Alcohol and Acetaldehyde in the Serum (Headspace Gas Chromatography)

(1) Detection conditions: 0.2 ml headspace sample was run in FID detector when sample injector and detector were heated to 140° C., the column oven was gradually heated from 35° C. to 70° C. and the carrier gas (N2), H2 and air were at a flow rate of 20, 50 and 500 Ml/min respectively.

(2) Sample analysis: 100 μl experimental animal serum sample or 100 μl ethanol/acetaldehyde standard prepared as described above were added into corresponding headspace bottles, which were then incubated at 70° C. in an incubator for 20 minutes, and 0.2 ml headspace gas was extracted and injected into chromatograph for analysis.

(3) Detection results: FIG. 5 shows one hour after fed with ethanol, the remaining ethanol content and acetaldehyde content in rats fed with engineered bacteria was less than half of that in rats fed with control bacteria. The trend line shows that the metabolic rate of ethanol and acetaldehyde in rats fed with engineered bacteria was significantly faster than that in rats fed with control bacteria.

Variation of ethanol content: 1 hour after fed with ethanol, the ethanol content in blood of control bacteria group and engineered bacteria group were 53.65±17.88 μM and 20.76±8.39 μM respectively. 2.5 hour after fed with ethanol, the ethanol content in blood of the above groups were 25.86±17.19 μM and 18.03±5.01 μM respectively. After 5 hours, the alcohol in blood of both groups returned to a normal level.

Variation of acetaldehyde content: 1 hour after fed with ethanol, the acetaldehyde content in blood of control bacteria group and engineered bacteria group were 8.12±1.20 μM and 4.23±1.39 μM respectively. 2.5 hour after fed with ethanol, the acetaldehyde content in blood of the above groups were 3.34±0.19 μM and 2.12±0.81 μM respectively. After 5 hours, the acetaldehyde in blood of both groups returned to a normal level.

1.-54. (canceled)

55. A genetically engineered probiotic intestinal bacterium comprising an exogenous expression cassette comprising a nucleotide sequence that encodes acetaldehyde dehydrogenase, wherein
the probiotic intestinal bacterium is Escherichia coli strain Nissle 1917 (EcN);
the acetaldehyde dehydrogenase is a naturally-occurring AcoD from Cupriavidus necator; the acetaldehyde dehydrogenase comprises an amino acid sequence of SEQ ID NO: 1, or an amino acid sequence having at least 80% sequence identity thereof yet retaining substantial activity in oxidizing aldehydes;
the nucleotide sequence that encodes the acetaldehyde dehydrogenase has been codon-optimized for expression in EcN, and optionally, the codon-optimized nucleotide sequence comprises a sequence of SEQ ID NO: 111 or a homologous sequence thereof having at least 80% sequence identity.

56. The genetically engineered probiotic intestinal bacterium of claim 55, wherein the expression cassette further comprises one or more regulatory elements comprising one or more elements selected from the group consisting of: a promoter, a ribosome binding site (RBS), a terminator, cistron, and any combination thereof.

57. The genetically engineered probiotic intestinal bacterium of claim 56, wherein the promoter is a constitutive promoter, preferably, the constitutive promoter comprises SEQ ID NO: 10; or an inducible promoter, preferably, the inducible promoter comprises an anaerobic inducible promoter, optionally, a nucleotide sequence of SEQ ID NO: 53.

58. The genetically engineered probiotic intestinal bacterium of claim 56, wherein the RBS comprises a nucleotide sequence selected from the group consisting of SEQ ID NOs: 65-67 and homologous sequences thereof having at least 80% sequence identity.

59. The genetically engineered probiotic intestinal bacterium of claim 56, wherein the terminator is T7 terminator.

60. The genetically engineered probiotic intestinal bacterium of claim 56, wherein the cistron is BCD2; preferably, the cistron comprises a nucleotide sequence of SEQ ID NO: 62 or homologous sequences thereof having at least 80% sequence identity.

61. The genetically engineered probiotic intestinal bacterium of claim 55, wherein the exogenous expression cassette is integrated in the genome of the genetically engineered probiotic intestinal bacterium.

62. The genetically engineered probiotic intestinal bacterium of claim 55, which expresses Chaperone proteins dnaK, dnaJ and grpE or groES, groEL and tig.

63. The genetically engineered probiotic intestinal bacterium of claim 55, further comprising at least one inactivation or deletion in an auxotroph-related gene, preferably, the probiotic intestinal bacterium is an auxotroph for one or more substances selected from the group consisting of thymidine, uracil, leucine, histidine, tryptophan, lysine, methionine, adenine, and non-naturally occurring amino acid.

64. A composition comprising the genetically engineered probiotic intestinal bacterium of claim 55, and a physiologically acceptable carrier.

65. The composition of claim 64, wherein the composition is edible, preferably, the composition is a food supplement.

66. The composition of claim 64, wherein the composition further comprises one or more physiologically acceptable carrier selected from lactic acid fermented foods, fermented dairy products, resistant starch, dietary fibers, carbohydrates, fat, oil, flavoring agent, seasoning agent, proteins and glycosylated proteins, water, capsule filler, and a gummy material.

67. The composition of claim 64, wherein the genetically-engineered microorganism is a live cell.

68. The composition of claim 64, wherein the composition is a finished food product, a powder, a granule, a tablet, a capsule, or a liquid.

69. A method for preventing and/or treating an alcohol hangover in a subject in need thereof, comprising administering to the gut of the subject an effective amount of the genetically engineered probiotic intestinal bacterium of claim 55.

70. The method of claim 69, wherein the subject is deficient in one or more aldehyde dehydrogenases.

71. The method of claim 69, wherein the composition is administered before, during, or after consumption of alcohol.

72. The method of claim 69, wherein the subject is a carrier of ALDH2 variant alleles.
